(19) United States
(12) Patent Application Publication
(10) Pub. No.: US 2026/0283701 A1
(43) Pub. Date: Sep. 24, 2026

(54) OPTICAL TRACKING SYSTEMS

(71) Applicant: BEYEONICS SURGICAL LTD., Haifa (IL)

(72) Inventor: Rani BEN-YISHAI, Haifa (IL)

(21) Appl. No.: 19/475,902

(22) PCT Filed: Apr. 18, 2024
(86) PCT No.: PCT/IL2024/050389

§ 371 Date: Oct. 16, 2025

Related U.S. Application Data
(60) Provisional application No. 63/460,658, filed on Apr. 20, 2023.

(51) Int. Cl. A61B 34/20; A61B 34/10; A61B 90/00; A61B 90/30; A61B 90/50

(52) U.S. Cl. CPC A61B 34/20; A61B 34/10; A61B 90/30; A61B 90/36; A61B 90/50; A61B 2034/105; A61B 2034/2055; A61B 2034/2063; A61B 2090/309; A61B 2090/365; A61B 2090/502

(57) ABSTRACT

Disclosed herein is an optical tracking system for determining a relative pose between a first coordinate system, which is associated with first and second tracking units configured to be fixed relative to one another; and a second coordinate system, which is associated with a third optical tracking unit, wherein each of the optical tracking units includes an optical sensor and a visual indicator. Further provided are uses of the system, and methods for determining relative pose between objects associated with the tracking units.

### TECHNICAL FIELD

The present disclosure relates generally to optical tracking systems.

### BACKGROUND

Optical tracking systems utilize data obtained by one or more optical sensors, included therein, for the real-time determination of the positions, and optionally the orientations, of one or more objects. Typically, optical sensors are employed to image discrete visual indicators, such as light emitting diodes (LEDs) or reflectors, thereby facilitating tracking the object. In inside-out (“in-out”) tracking the optical sensors are located on a tracked object and the visual indicators are fixed in known locations in a surrounding environment, while in outside-in (“out-in”) tracking the optical sensors may be stationary and the visual indicators are fixed in known locations relative to a tracked object.

U.S. Pat. No. 9,618,621 is directed to a compact inside-out/outside-in (“in-out/out-in”) optical tracker for tracking a relative position and orientation, or pose, between two optical tracker sensors.

Generally, for in-out/out-in tracking, a total of at least two sensors and three visual indicators are required. Two of the visual indicators are fixed relative to one of the sensors, and the third visual indicator is fixed relative to the second sensor (e.g., the locations of the visual indicators relative to their respective sensors are known). Typically, each of the sensors together with its corresponding visual indicator or indicators are incorporated within a tracking unit—a first tracking unit comprising a sensor and two visual indicators, and a second tracking unit comprising a sensor and one visual indicator. Each of the tracking units is fixed relative to one of two objects, generally facing each other. This allows for detecting three directions from the sensors to the visual indicator (or indicators) of the opposite tracking unit—one direction from the sensor of the first tracking unit, and two directions from the sensor of the second tracking unit. Two equations can be derived from each direction, resulting with a total of six equations, thereby facilitating the calculation of the six degrees of freedom (DOF) representing the relative pose between the two tracking units.

It is noted that the distance between the two visual indicators of the first tracking unit, and specifically the ratio between this distance and the distance between the two tracking units (or between the two tracked objects), can affect the accuracy of the pose determination (e.g., larger distance between the two visual indicators will result with higher accuracy). Accordingly, there may be a restriction on the minimal distance between the two visual indicators, and consequently, on the minimal size of the tracking unit that comprises the sensor and the two visual indicators. There may also be other limitations due to the size or shape of the object that the tracking unit is configured to be fixed to. Therefore, the distance between the two visual indicators may be selected by taking into consideration, among other criteria, both the characteristic distance between the two tracked objects and the size or shape of the object. As a non-limiting example, a tracking unit comprising a sensor and a single LED can have a largest dimension of less than about 2 cm, whereas a tracking unit comprising two LEDs can have a largest dimension of about 10 cm (corresponding to the selected distance between the two LEDs). In this example, the distance between the two tracking units (e.g., when employed for tracking) can be between about 40 and 80 cm (e.g., the distance between the tracking units dynamically changes when the two tracked objects move relative to one another). There remains a need in the art for enhanced optical tracking systems that allow tracking between two or more objects in flexible settings.

### SUMMARY

Aspects of the disclosure, according to some embodiments thereof, relate to optical tracking systems for determining the relative position and orientation (pose), between two or more objects. More specifically, but not exclusively, aspects of the disclosure, according to some embodiments thereof, relate to inside-out/outside-in optical tracking systems, wherein to each tracked object one or more optical tracking units (also referred to herein as “tracking units” or “tracker units”), each including at least one optical sensor and at least one visual indicator, are attached. Further described herein is the use of the optical tracking systems in various settings, including, for example, but not limited to, visor guided surgery (VGS) settings, wherein a see-through head mounted display (HMD) is employed to augment the surgeon's view of the patient, so as to allow “seeing” internal anatomical features of the patient (e.g., as if parts of the patient's body were transparent). According to some embodiments of the current disclosure, each of the disclosed optical tracking units includes an optical sensor and a visual indicator. In some embodiments, wherein the relative pose between a pair of objects is to be determined, two tracking units are fixed relative to the first object and one tracking unit is fixed relative to the second object. The sensor of the tracking unit fixed to the second object may be used to image the visual indicators of the tracking units fixed to the first object (e.g., light emitted or reflected by the visual indicators is detected, or “imaged”, by the optical sensor). Either (or both) of the two optical sensors of the two tracking units fixed to the first object may be used to image the visual indicator of the tracking unit fixed to the second object. According to some embodiments, the two tracker units that are fixed relative to the first object may have partially overlapping field-of-views (i.e. the FOVs of the optical sensors thereof partially overlap). According to some embodiments, to allow the calculation of the relative pose between the coordinate system associated with the first object and the coordinate system associated with the second object based on data from the sensors, first the relative pose between the two tracking units that are fixed relative to the first object is determined, as will be described further below.

Thus, according to an aspect of some embodiments, there is provided an optical tracking system including a first (optical) tracking unit, a second (optical) tracking unit, a third (optical) tracking unit, and one or more processors (for example, included in computer hardware) communicatively associated with the tracking units. Each of the tracking units includes an optical sensor and a visual indicator. The first and second tracking units are configured to be fixed relative to one another (e.g. both may be fixed onto a same object). The one or more processors are configured to determine, when the first and second tracking units are fixed relative to one another, a relative pose between a first coordinate system and a second coordinate system. The first coordinate system is associated with the first and second tracking units, and the second coordinate system is associated with the third tracking unit. The determination of the relative pose between the first and second coordinate systems is performed by the one or more processors based at least on:
information indicative of a pose of each of the first and second tracking units in the first coordinate system;
a first data set indicative of at least one direction in the first coordinate system from the optical sensor of the first tracking unit and/or the optical sensor of the second tracking unit towards the visual indicator of the third tracking unit; and
a second data set indicative of two directions in the second coordinate system from the optical sensor of the third tracking unit towards the visual indicator of the first tracking unit and the visual indicator of the second tracking unit.

According to some embodiments, each of the first data set and the second data set includes at least one of: blob data and image data.

According to some embodiments, the one or more processors are further configured to determine, based on the first and second data sets, at least one direction from at least one of the optical sensors towards at least one of the visual indicators.

According to some embodiments, the one or more processors are further configured to select from the first and second tracking units, whether both thereof, or which thereof, are used to obtain the first data set.

According to some embodiments, each of the optical sensors is selected from a pixel array sensor, a CMOS sensor, a CCD sensor, a pair of linear sensor arrays, a 2D lateral effect position sensor, a photodiode array position sensor, a segmented photodetector, a quadrant position-sensing detector, a position-sensitive detector, and a light sensitive element. Each possibility is a separate embodiment.

According to some embodiments, each of the visual indicators may include a light emitting diode (LED) or a reflector.

According to some embodiments, the tracking units may be fabricated to be similar with respect to one or more of size, dimensions, type of optical sensor, type of visual indicator, type of communication unit, communication protocol, and/or functionality. Each possibility is a separate embodiment.

According to some embodiments, the first and second tracking units are configured to be fixed relative to one of: a head mounted display (HMD), a surgical microscope, a surgical exoscope, a surgical robot, a surgical robotic arm, an imaging device, a surgical light. Each possibility is a separate embodiment.

According to some embodiments, the third tracking unit is configured to be fixed relative to one of: a hand-held tool, a subject, an anatomical site, a camera, an endoscope, a laparoscope. Each possibility is a separate embodiment.

According to some embodiments, the information indicative of the relative pose between the first and second tracking units is predetermined.

According to some embodiments, the first and second tracking units are permanently attached to an object.

According to some embodiments, the one or more processors are further configured to implement a calibration stage wherein the relative pose between the first and second tracking units is computed.

According to some embodiments, the information indicative of a pose of each of the first and second tracking units in the first coordinate system comprises previously acquired first and second data sets.

According to some embodiments, the information indicative of a pose of each of the first and second tracking units in the first coordinate system is based on an accurate mounting mechanism.

According to some embodiments, there is provided an optical-based tracking method for determining a relative pose between (i) a first coordinate system, which is associated with a first tracking unit and a second tracking unit, and (ii) a second coordinate system, which is associated with a third tracking unit, each of the tracking units comprises an optical sensor and a visual indicator, the method includes:
using the first tracking unit and/or the second tracking unit to obtain a first data set indicative of at least one direction in the first coordinate system from the optical sensor of the first tracking unit and/or the optical sensor of the second tracking unit towards the visual indicator of the third tracking unit;
using the third tracking unit to obtain a second data set indicative of two directions in the second coordinate system from the optical sensor of the third tracking unit towards the visual indicator of the first tracking unit and the visual indicator of the second tracking unit; and
determining the relative pose between the first coordinate system and the second coordinate system based at least on the first and second data sets and information indicative of a pose of each of the first and second tracking units in the first coordinate system;
wherein the first and second tracking units are fixed relative to one another.

According to some embodiments, each of the first data set and the second data set includes at least one of: blob data and image data.

According to some embodiments, the method may further include determining, based on the first and second data sets, at least one direction from at least one of the optical sensors towards at least one of the visual indicators.

According to some embodiments, the method may further include selecting from the first and second tracking units, whether both thereof, or which thereof, are used to obtain the first data set.

According to some embodiments, each of the optical sensors is selected from a pixel array sensor, a CMOS sensor, a CCD sensor, a pair of linear sensor arrays, a 2D lateral effect position sensor, a photodiode array position sensor, a segmented photodetector, a quadrant position-sensing detector, a position-sensitive detector, and a light sensitive element.

According to some embodiments, each of the visual indicators may include a light emitting diode (LED) or a reflector.

According to some embodiments, the first and second tracking units are configured to be fixed relative to one of: a head mounted display (HMD), a surgical microscope, a surgical exoscope, a surgical robot, a surgical robotic arm, an imaging device, a surgical light.

According to some embodiments, the third tracking unit is configured to be fixed relative to one of: a hand-held tool, a subject, an anatomical site, a camera, an endoscope, a laparoscope.

According to some embodiments, the information indicative of the relative pose between the first and second tracking units is predetermined.

According to some embodiments, the first and second tracking units are permanently attached to an object.

According to some embodiments, the method may further include a calibration stage, wherein the relative pose between the first and second tracking units is computed.

According to some embodiments, information indicative of a pose of each of the first and second tracking units in the first coordinate system comprises previously acquired first and second data sets. According to some embodiments, information indicative of a pose of each of the first and second tracking units in the first coordinate system is based on an accurate mounting mechanism.

According to some embodiments, there is provided an optical tracking system including a first, second object, third, and fourth optical tracking units, and one or more processors; wherein each of the tracking units comprises an optical sensor and a visual indicator; wherein the first and second tracking units are configured to be fixed relative to one another; wherein the one or more processors are configured to determine, when the first and second tracking units are fixed relative to one another, a relative pose between a first coordinate system, which is associated with the first and second tracking units, and each of a second coordinate system, which is associated with the third tracking unit, and a third coordinate system, which is associated with the fourth tracking unit, based at least on: information indicative of a pose of each of the first and second tracking units in the first coordinate system; a first data set indicative of a direction in the first coordinate system from the optical sensor of the first tracking unit towards the visual indicator of the third tracking unit; a second data set indicative of a direction in a first coordinate system, from an optical sensor of the second tracking unit towards a visual indicator of a fourth tracking unit; a third data set indicative of two directions in the second coordinate system from the optical sensor of the third tracking unit towards the visual indicator of the first tracking unit and towards the visual indicator of the second tracking unit; and a fourth data set indicative of two directions in the third coordinate system from the optical sensor of the fourth tracking unit towards the visual indicator of the first tracking unit and towards the visual indicator of the second tracking unit.

According to some embodiments, there is provided an optical-based tracking method for determining a relative pose between (i) a first coordinate system, which is associated with a first tracking unit and a second tracking unit, the first and second tracking units being fixed relative to one another, and (ii) each of a second coordinate system, which is associated with a third tracking unit, and a third coordinate system, which is associated with a fourth tracking unit, the method includes: using the first unit to obtain a first data set indicative of a direction in the first coordinate system from an optical sensor of the first tracking unit towards a visual indicator of the third tracking unit; and from an optical sensor of the second tracking unit towards a visual indicator of the fourth tracking unit; using the second tracking unit to obtain a second data set indicative of a direction in the first coordinate system from an optical sensor of the second tracking unit towards a visual indicator of the fourth tracking unit; using the third tracking unit to obtain a third data set indicative of two directions in the second coordinate system from an optical sensor of the third tracking unit towards visual indicators of the first and second tracking units; using the fourth tracking unit to obtain a fourth data set indicative of two directions in the third coordinate system from an optical sensor of the fourth tracking unit towards the visual indicators of the first and second tracking units; and determining the relative pose between the first coordinate system and each the second and third coordinate systems based at least on the first, second, and third data sets and information indicative of a pose of the first and second tracking units in the first coordinate system.

According to some embodiments, there is provided a system for visualizing a surgical field, the system includes: a camera assembly mounted on a maneuverable arm and configured to obtain images of the surgical field; a see-through head mounted display (HMD) configured to display the images of the surgical field, and further configured to allow a direct view of the surgical field; at least one camera tracking unit configured to be fixed relative to the camera assembly; at least one HMD tracking unit configured to be fixed relative to the HMD; at least one patient tracking unit configured to be fixed relative to an anatomical site located at or near the surgical field; and a processor configured to determine, based on data acquired from at least two of the at least one camera tracking unit, the at least one HMD tracking unit, and the at least one patient tracking unit, at least one of: a relative pose between the camera assembly and the anatomical site, a relative pose between the HMD and the anatomical site, and a relative pose between the HMD and the camera assembly; wherein each of the at least one camera tracking unit, the at least one HMD tracking unit, and the at least one patient tracking unit comprises at least one optical sensor and at least one visual indicator; wherein the HMD is further configured to display a first guidance information, generated based on one or more of the determined relative poses, as an overlay on the images of the surgical field; and wherein the HMD is further configured to display a second guidance information, generated based on one or more of the determined relative poses, as an overlay on the direct view of the surgical field.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

Unless specifically stated otherwise, as apparent from the disclosure, it is appreciated that, according to some embodiments, terms such as “processing”, “computing”, “calculating”, “determining”, “estimating”, “assessing”, “gauging” or the like, may refer to the action and/or processes of a computer or computing system, or similar electronic computing device, that manipulate and/or transform data, represented as physical (e.g. electronic) quantities within the computing system's registers and/or memories, into other data similarly represented as physical quantities within the computing system's memories, registers or other such information storage, transmission or display devices.

Embodiments of the present disclosure may include apparatuses for performing the methods herein. The apparatuses may be specially constructed for the desired purposes or may include a general-purpose computer(s) selectively activated or reconfigured by a computer program stored in the computer. Such a computer program may be stored in a computer readable storage medium, such as, but not limited to, any type of disk including floppy disks, optical disks, CD-ROMs, magnetic-optical disks, read-only memories (ROMs), random access memories (RAMs), electrically programmable read-only memories (EPROMs), electrically erasable and programmable read only memories (EEPROMs), magnetic or optical cards, flash memories, solid state drives (SSDs), or any other type of media suitable for storing electronic instructions, and capable of being coupled to a computer system bus.

The processes and displays presented herein are not inherently related to any particular computer or other apparatus. Various general-purpose systems may be used with programs in accordance with the teachings herein, or it may prove convenient to construct a more specialized apparatus to perform the desired method(s). The desired structure(s) for a variety of these systems appear from the description below. In addition, embodiments of the present disclosure are not described with reference to any particular programming language. It will be appreciated that a variety of programming languages may be used to implement the teachings of the present disclosure as described herein.

Aspects of the disclosure may be described in the general context of computer-executable instructions, such as program modules, being executed by a computer. Generally, program modules include routines, programs, objects, components, data structures, and so forth, which perform particular tasks or implement particular abstract data types. Disclosed embodiments may also be practiced in distributed computing environments where tasks are performed by remote processing devices that are linked through a communications network. In a distributed computing environment, program modules may be located in both local and remote computer storage media including memory storage devices.

### BRIEF DESCRIPTION OF THE FIGURES

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not drawn to scale. Moreover, two different objects in the same figure may be drawn to different scales. In particular, the scale of some objects may be greatly exaggerated as compared to other objects in the same figure.

In the figures:

FIG. 1 presents a block diagram of an optical tracking system for determining a relative position and orientation (pose) between a first object and a second object, according to some embodiments;

FIGS. 2A-2D present schematic illustrations of on optical tracking system including a first and second optical tracking units positioned on a first object, and a third optical tracking unit positioned on a second object, according to some embodiments;

FIG. 3 shows a flowchart of an optical tracking-based method for determining a relative pose between a first coordinate system and a second coordinate system, according to some embodiments;

FIG. 4 presents a block diagram of an optical tracking system for determining a relative pose between a first object, a second object and a third object, according to some embodiments;

FIGS. 5A-5B present a schematic illustration of on optical tracking system including a first and second optical tracking units positioned on a first object, a third optical tracking unit positioned on a second object, and a fourth optical tracking unit positioned on a third object, according to some embodiments;

FIG. 6 presents a flowchart of an optical tracking-based method for determining a relative pose between a first coordinate system and each of a second coordinate system, and a third coordinate system, according to some embodiments;

FIG. 7 presents a schematic illustration of a visual guided surgery (VGS) system utilizing an optical tracking system for determining a relative pose between at least a first and second objects, according to some embodiments;

FIG. 8 presents a schematic illustration of an integrated visualization and guidance system utilizing an inside-out/outside-in optical tracking system for determining a relative pose between several objects, according to some embodiments; and

FIG. 9 presents a schematic illustration of an integrated visualization and guidance system utilizing an inside-out/outside-in optical tracking system for determining a relative pose between several objects, according to some embodiments.

### DETAILED DESCRIPTION

The principles, uses, and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout.

In the description and claims of the application, the words “include” and “have”, and forms thereof, are not limited to members in a list with which the words may be associated.

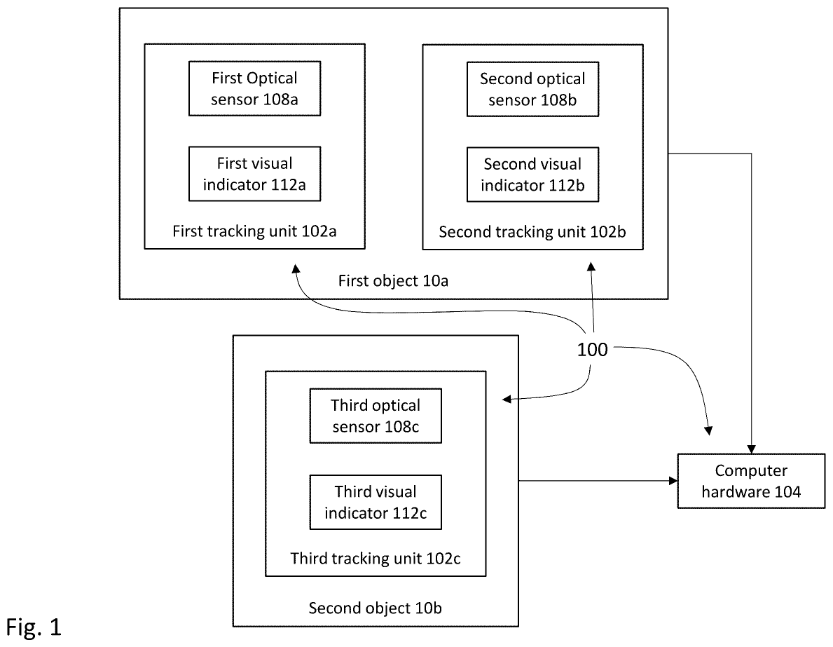

According to an aspect of some embodiments, there is provided an optical tracking system for determining a relative position and orientation, or pose, between a first object and a second object. FIG. 1 presents a block diagram of such an optical tracking system 100, according to some embodiments. System 100 includes three optical tracking units 102 (“tracking units 102” for short): a first tracking unit 102a, a second tracking unit 102b, and a third tracking unit 102c. System 100 further includes computer hardware 104 communicatively associated with tracking units 102.

Also shown are a first object 10a and a second object 10b, whose relative pose is to be determined by system 100. Each of first tracking unit 102a and second tracking unit 102b is fixed (i.e. secured) relative to first object 10a. Third tracking unit 102c is fixed (i.e. secured) relative to second object 10b. It is to be understood that first object 10a and second object 10b are not included in system 100.

Tracking units 102 include three optical sensors 108 and three visual indicators 112, respectively: first tracking unit 102a includes a first optical sensor 108a and a first visual indicator 112a, second tracking unit 102b includes a second optical sensor 108b and a second visual indicator 112b, and third tracking unit 102c includes a third optical sensor 108c and a third visual indicator 112c. According to some embodiments, at least some of the tracking units are positioned opposite thereto. For example, as shown in FIG. 1, first tracking unit 102a and second tracking unit 102b are opposite to third tracking unit 102c and vice versa.

According to some embodiments, each of optical sensors 108 may include a pixel array sensor (such as a CMOS sensor or a CCD sensor), a pair of linear sensor arrays, a 2D lateral effect position sensor, a photodiode array position sensor, a segmented photodetector (such as a quadrant position-sensing detector), or any other light-sensitive or position-sensitive components. As used herein, the term “optical sensor” is to be understood in an expansive manner as encompassing not only a light sensitive element (e.g. a pixel array sensor or a photodiode array) but also additional optical elements, such as lenses, filters, and stops (e.g. aperture stops, field stops), which may be used to focus, filter, and/or control the amount, and/or the FOV, of the light incident on the light sensitive element.

According to some embodiments, one or more of visual indicators 112 may emit or reflect light in any suitable wavelength that can be sensed by the optical sensor, such as, for example, light in the infrared range. In some exemplary embodiments, one or more of the visual indicators may be an infrared light emitting diode (LED). According to some embodiments, one or more of visual indicators 112 may be configured to allow controllably emitting or reflecting light in other wavelength ranges.

According to some embodiments, each of visual indicators 112 may be or include a reflector. According to some such embodiments, each of tracking units 102 may further include a photoemitter configured to illuminate the field-of-view (FOV) of the respective optical sensor. According to some embodiments, the photoemitter may form part of (in the sense of being included in) the respective optical sensor (e.g. the photoemitter of first tracking unit 102 may be included in first optical sensor 108a). Additionally, or alternatively, according to some embodiments (wherein at least some of the visual indicators include a reflector), system 100 may further include one or more photoemitters external to tracking units 102, which are configured to illuminate one or more of the visual indicators. For example, one or more photoemitters can be fixed to the first and second objects. As another example, one or more photoemitters can be stationary.

According to some embodiments, when the visual indicators are light emitting (e.g., when visual indicators are LEDs), they can be employed to emit pulses of light that are synchronized with the integration time of the opposite optical sensors (e.g., optical sensors in opposite tracking units). Similarly, when the visual indicators are reflectors, the light that they reflect can be emitted in pulses that are synchronized with the integration time of the optical sensors that are opposite to the visual indicators.

According to some embodiments, when the visual indicators are light emitting and when the optical sensors are pixel array sensors, visual indicator 112a in tracking unit 102a and visual indicator 112b in tracking unit 102b can be employed to simultaneously emit pulses of light that are synchronized with the integration time of optical sensor 108c in tracking unit 102c. According to some embodiments, when the visual indicators are light emitting and when the optical sensors are photodiode-based position sensors, visual indicator 112a in tracking unit 102a and visual indicator 112b in tracking unit 102b can be employed in different times (e.g., non-simultaneously) to emit pulses of light that are synchronized with the integration times of optical sensor 108c in tracking unit 102c. For instance, in these embodiments, the two visual indicators can be employed consecutively and the optical sensor can be operated in synchronization with each of them.

As used herein, the term “field-of-view” (FOV), when employed with respect to a tracking unit (e.g. first tracking unit 102a), is to be understood as referring to the FOV of the optical sensor (e.g. first optical sensor 108a) of the tracking unit. The FOV of an optical sensor is to be understood as referring to the effective FOV thereof in the sense of being determined not, or not only, by the light sensitive element but by, or additionally by, optical elements, which may impact the effective FOV, such as a field stop.

According to some embodiments, one or more of tracking units 102 may additionally include a battery (not shown) configured to power operation of components of the tracking unit. According to some embodiments, the battery may be rechargeable and/or replaceable. According to some embodiments, the battery is not rechargeable and/or replaceable. According to some embodiments, wherein the tracking unit is integrated into an object requiring power, which is to be tracked (e.g. in embodiments wherein first and tracking second units 102a and 102b are integrated into object 10a), the tracking unit may be powered by the same power supply used to power the object (e.g., in these embodiments the tracking unit does not include a battery).

According to some embodiments, each of tracking units 102 may additionally include a processing and control circuitry (not shown), which may include, for example, one or more of a field-programmable gate array (FPGA), an application-specific integrated circuit (ASIC), and a central processing unit (CPU), and, more generally, a system on a chip (SoC). The processing and control circuitry may be configured, for example, to control the exposure time (e.g., integration time) of the respective optical sensor, control the location and size of one or more regions of interest (ROIs) to be output by the optical sensor and read the values of the pixels in the ROIs once the exposure is complete (e.g. when the optical sensor is a pixel array sensor), actuate the respective visual indicator (in embodiments wherein the visual indicator is light emitting, such as when the visual indicator is an LED), and so on. According to some embodiments, when the optical sensor is a pixel array sensor, the processing and control circuitry may additionally be configured to process the pixel values that are read from the optical sensor, for instance, to derive binary large object (“blob”) data and/or histogram data. According to some embodiments, the processing and control circuitry may be configured to determine the position of the center of a blob (e.g., a 2D position within the pixel array). According to some embodiments, thanks to the optical characteristics of the light sensitive element and the optical elements of the optical sensor, the optical sensor detects light only in a range of wavelengths around the wavelength that is emitted (or reflected) by the visual indicators. It is noted that in these embodiments, when the optical sensor is a pixel array sensor, the visual indicators can appear in the image acquired by the sensor as discrete blobs.

According to some embodiments, each of tracking units 102 may additionally include a communication unit (not shown). In some embodiments, the communication unit is a wireless communication unit including, for example, a Wi-Fi module, a Bluetooth module, and/or a Bluetooth Low Energy module. The communication unit may be communicatively associated with computer hardware 104. The communication unit may be commanded by the processing and control circuitry, which, in particular, may select which of the sensor-acquired data (optionally, after processing thereof) is to be sent to computer hardware 104. In some embodiments, the communication unit may include the processing and control circuitry in addition to or instead of the aforementioned processing and control circuitry. In some embodiments, when the optical sensor is a pixel-array sensor, the sensor-acquired data includes image data (e.g., the image, or part of the image acquired by the sensor). In some embodiments, when the optical sensor is a pixel-array sensor, the sensor-acquired data includes blob data (e.g., the processed image, a processed ROI within the image, or several processed ROIs within the image).

According to some embodiments, each of tracking units 102 may additionally include an inertial measurement unit (IMU) comprising one or more accelerometers and/or one or more gyroscopes, and optionally also one or more magnetometers. In some embodiments, based on readings from the IMU, tracking system 100 may determine predicted pose estimations. Such estimations may be employed to compensate for latency between the time light is detected by optical sensors in any of tracking units 102, and the time when information based on the determined pose is provided to a user. For example, when the information is provided via an optical see-through HMD (e.g., first object 10a) as an overlay on the direct view of the surrounding world, a predicted pose may allow an overlay to appear as fixed relative to a hand-held object (e.g., second object 10b). In some embodiments, based on readings from the IMU, tracking system 100 may determine whether an object is stationary or not. For example, tracking system 100 may determine whether a hand-held tool, such as a surgical tool 10b, is picked up (e.g., picked up by a nurse from a sterile tray) and handed to a surgeon donning HMD 10a. This can allow tracking system 100, for instance, to initiate tracking between HMD 10a and tool 10b. In some embodiments, based on readings from the IMU, tracking system 100 may determine that an object was impacted. For example, when second object 10b is a patient and third tracking unit 102c is fixed to a spinous process of the patient, tracking system 100 may determine that tracking unit 102c was impacted (e.g., unintentionally) and initiate verification and/or corrective steps. For instance, tracking system 100 may alert the surgeon that tracking unit 102c was impacted, thereafter the surgeon may verify that a registration between the tracking unit and the patient anatomy is still valid (registration is described further below).

According to some embodiments, each of tracking units 102 may additionally include a memory (e.g., a non-volatile memory). The memory may be separate from the processing and control circuitry or forming a part thereof. The memory may store, for example, mechanical data (e.g. the location of the visual indicator and the optical sensor in the tracking unit's coordinate system), calibration data (such as the mapping between blob locations in the pixel array sensor and directions in space (e.g., in the tracking unit's coordinate system)), manufacturing data, and the like, or combinations thereof. In some embodiments, at least part of the data stored in the memory may be sent to computer hardware 104 when the tracking unit is switched on or upon request.

According to some embodiments, the stored calibration data may specify or be indicative of the relative pose between first tracking unit 102a and second tracking unit 102b. These data and/or additional calibration data may be stored in the memory of tracking units 102a and 102b and/or in the memory of an object that tracking units 102a and 102b are fixed to (for example, a head mounted display (HMD)). According to some embodiments, such as embodiments wherein tracking units 102a and 102b are permanently installed on, or integrated into, first object 10a, the calibration data may specify, or be indicative of, the relative poses between each of tracking units 102a and 102b and first object 10a. Put differently, in such embodiments the calibration data can specify, or be indicative of, the poses of each of first tracking unit 102a, second tracking unit 102b, and first object 10a within a first coordinate system in which each of first tracking unit 102a, second tracking unit 102b, and first object 10a is stationary (e.g., the first coordinate system is associated with the first and second tracking units and the first object). Similarly, according to some embodiments, such as embodiments wherein third tracking unit 102c is permanently installed on, or integrated into, second object 10b, third tracking unit 102c and/or second object 10b may include a memory storing calibration specifying, or being indicative of, among others, the relative pose between third tracking unit 102c and second object 10b. Put differently, in such embodiments the calibration data (stored in the memory of third tracking unit 102c and/or second object 10b) can specify, or be indicative of, the poses of each of third tracking unit 102c and second object 10b within a second coordinate system in which each of third tracking unit 102c and second object 10b is stationary.

According to some embodiments, tracking units that are required to be sterile (e.g., such as a tracking unit that is fixed to a patient or a tracking unit that is fixed to a surgical instrument) can be sterilized prior to use (for example, using an autoclave between procedures). According to some embodiments, tracking units that are required to be sterile are covered during surgery by a sterile cover. In these embodiments, the sterile cover can be transparent (e.g., transparent to the light emitted or reflected by the visual indicators and imaged by the optical sensors) or have a transparent window (or windows) for the optical sensor and the visual indicator. According to some embodiments, tracking units that are required to be sterile are provided as single use devices (e.g., disposable). In these embodiments, the tracking units can be pre-sterilized and provided in sterile packages.

According to some embodiments, each of tracking units 102 can be separated into two sub-assemblies. A first sub-assembly, the functional part of the tracking unit, can comprise the optical sensor and the visual indicator, and additional components that are required to be in their vicinity (such as, for example, various capacitors and resistors). The functional part of the tracking unit can optionally comprise also an IMU (e.g., a MEMS-based IMU) and/or a memory. The second sub-assembly can comprise all other components. According to some embodiments, the functional sub-assembly can be disposable, and the second sub-assembly can be reuseable. According to some embodiments, the second sub-assembly can include a rechargeable or replaceable battery. According to some embodiments, when the functional sub-assembly is disposable, it can be provided as a sterile part. According to some embodiments, when the functional sub-assembly is reuseable, it can be either sterilizable (e.g., by autoclave) or covered with a transparent sterile cover or with a sterile drape having an optical window (or windows) for the sensor and visual indicator. According to some embodiments, the second sub-assembly can be either sterilizable (e.g., by autoclave) or can be covered by a sterile cover (e.g., a sterile nylon sleeve) when connected to the functional sub-assembly during surgery. The two parts can be connected by a wire that can be part of the functional sub-assembly or part of the second sub-assembly.

The two sub-assemblies design can be advantageous for tracking units that are configured to be fixed to a patient. In some embodiments, the tracking unit can be fixed to the skin via adhesion. In these embodiments, the small size of the functional part of the tracking unit (e.g., the part comprising the optical sensor and the visual indicator) can allow attaching it to areas less susceptible to movement. In some embodiments, the tracking unit is fixed to a spinous process during an open (or mini-open) spine procedure. In these embodiments, the small size and weight of the functional part of the tracking unit can facilitate fixing it to fragile bones and can allow fixing a tracking unit to more than one vertebra. Tracking individual vertebrae can increase navigation accuracy as it can eliminate inaccuracy originating from inter-vertebra movement, and can also facilitate vertebrae alignment, for instance in deformative cases. In some embodiments, the tracking unit is fixed relative to the head of a patient. In these embodiments, the small size and weight of the functional part of the tracking unit can allow fixing it directly to the patient's head (e.g., to the skin via adhesion). This can be advantageous, for instance, during a bedside VGS procedure in an intensive care unit (e.g., procedures such as external ventricular drain placements or subdural hematoma drainages). In some embodiments, in various minimally invasive spine surgeries (such as anterior cervical discectomy and fusion procedures or lateral interbody fusion procedures), the tracking unit is fixed to relative to a vertebral body. In these embodiments, the small size and weight of the functional part of the tracking unit can facilitate its fixing to retractors and/or distractors that are fixed relative to the vertebrae. In all the above examples, only the functional part is required to be fixed to the tracked part of the anatomy, and the second part can be fixed elsewhere (e.g., either to the patient or to the surgical table, for example). By way of a non-limiting example, the size of a functional part comprising an optical sensor and an LED can be 20×15 millimeters. The advantages of the two sub-assembly design are not limited only to tracking units that are configured to be fixed to a patient, rather this design can also be advantageous for other tracking units, such as, for instance, tracking units that are configured to be fixed to a surgical tool.

According to some embodiments, the tracking units can be permanently attached to an object. According to some embodiments, the tracking units can be attached to an object “in the field”, just prior to use. Once attached, determining the alignment (i.e., the relative fixed pose) between the tracking unit and the object is typically required. In the case of permanent attachment, calibration (e.g., determining the alignment) is typically performed in the factory. In the case of field attachment, calibration is typically performed in the field. Alternatively, in both cases, calibration may not be required, and determining the alignment can be based on accurate mechanical mounting of the tracking units to the object. Typically, for tracking a relative pose between two objects according to the current disclosure, determining an alignment between the tracking unit/s and the object is required both when fixing two tracking units relative to a first object and when fixing a single tracking unit relative to the second object.

According to some embodiments, first tracking unit 102a and second tracking unit 102b are fixed one relative to the other (and optionally also relative to first object 10a) such that the FOVs thereof (i.e., the FOVs of first optical sensor 108a and second optical sensor 108b) partially overlap.

According to some embodiments, when two tracking units are fixed relative to one another (and optionally also relative to an object), the relative pose between them needs to be determined to allow the calculation of the relative pose between the joint coordinate system associated with the two tracking units and the coordinate system associated with a third tracking unit. Typically, the two tracking units are fixed relative to an object having an associated coordinate system. In these cases, the relative pose between each of the tracking units and the object (e.g., between the coordinate system associated with each of the tracking units and the coordinate system associated with the object) can be determined separately (e.g., by calibration or based on known mechanical mounting), and the relative pose between the two tracking units can then be derived. Alternatively, the relative pose between the two tracking units can be directly determined (e.g., by calibration or based on known mechanical mounting).

Generally, for in-out/out-in tracking, a total of at least two sensors and three visual indicators are required. Once the relative pose between the two tracking units is known, the location of the visual indicator of the first tracking unit relative to the sensor of the second tracking unit is known, and vice versa. Therefore, by employing at least one of the two sensors of the first and second tracking units, and the sensor of the third tracking unit, to image the three visual indicators, the six equations required for calculating of the six degrees of freedom (DOF) representing the relative pose between the coordinate system associated with the first and second tracking units and the coordinate system associated with the third tracking unit can be derived. It is noted that in some cases the set of equations can have two solutions, representing two poses rotated by about 180 degrees relative to one another, of the third tracking unit relative to the first and second tracking units (e.g., when the sensor is a pixel-array sensor, in these cases the system can incorrectly identify which of the two visual indicators is represented by each of the two blobs in the image). When the visual indicators are active (e.g., LEDs), this redundancy can be solved by separately controlling the two visual indicators. For example, the system can periodically (for instance once in every 60 cycles) turn off one of the visual indicators and verify that indeed the expected BLOB disappeared (e.g., when the sensor is a pixel-array sensor). When the visual indicators are passive (e.g., reflectors) the system can switch the sensor employment between the sensor of the first tracking unit and the sensor of the second tracking unit and verify that the determined pose stays unchanged (e.g., up to an allowed tolerance). Verifying the correct identification of visual indicators can be performed continuously, periodically, or only after a period without tracking.

In some embodiments, when two or more tracking units are attached to an object, it is permanent, and a calibration (e.g., determining the relative pose between the tracking units) may be done in the factory. In some embodiments, when two or more tracking units are attached to an object, they are attached in the field and a calibration can be facilitated by computer hardware 104. According to some embodiments, computer hardware 104 may thus be configured to compute the relative pose between first tracking unit 102a and second tracking unit 102b based on data acquired by each of tracking units 102 (e.g., following setup/installation of tracking units 102 and prior to commencing a surgical procedure). According to some embodiments, computer hardware 104 may be configured to compute the relative pose between first tracking unit 102a and second tracking unit 102b based on data acquired by tracking units 102a and 102b, and an additional tracking unit. According to some embodiments, computer hardware 104 may be configured to compute the relative pose between first tracking unit 102a and second tracking unit 102b based on data acquired by tracking units 102a and 102b, and an additional pair of tracking units that are fixed to one another and were previously calibrated (e.g., the relative pose between the two tracking units of the additional pair is known). Examples of such calibration processes that can also be performed in the field are given further below. According to some other embodiments, the computer hardware may be configured to verify, or determine to higher accuracy, the relative pose between first tracking unit 102a and second tracking unit 102b based on a previously determined relative pose there between and current data acquired by each of tracking units 102 (e.g. following setup/installation of tracking units 102 and prior to commencing a surgical procedure).

In some embodiments, the calibration in the case of two or more tracking units can typically include the alignment of the tracking units one relative to the other, and also the alignment of the tracking units relative to the object. However, as mentioned above, the alignment to the object is not mandatory and can depend on the application and/or the tracked objects.

Typically, when attaching a tracking unit to a surgical tool, either a verification or a calibration of the alignment between the tracking unit and the tool is required. Both the verification and the calibration can be based on some prior estimation of the alignment and/or knowledge on the shape of the tool. Field calibration may be typically performed by computer hardware 104. However, the attachment of a tracking unit to a tool can also be permanent and a calibration can be pre-performed in the factory, and not by computer hardware 104. For attachment of a tracking unit (or units) to a subject (patient), the calibration (e.g., determining the pose between the tracking unit and the patient anatomy) is typically referred to as “registration”. The registration can result with having both the tracking unit and the patient's image data (such as, for example, preoperative and/or intraoperative CT or MRI scans) being registered to the patient's anatomy, thus allowing the system to display the location of a tracked tool relative to the image data.

In some embodiments, the relative pose between the tracking units and the respective objects is not known in full. In these embodiments, system 100 can determine only a partial pose between the objects, although the full 6-DOF pose between said two tracking units and said third tracking unit (e.g., between the joint coordinate system for both the first and second tracking units and the coordinate system of the third tracking unit) can be determined. For example, in some embodiments, third tracking unit can be fixed to a surgical instrument with an adapter that allows the tracking unit to freely swivel around the tool. In these embodiments, only 5-DOF of the relative pose between the third tracking unit and the tool are known, and consequently, only 5-DOF of the relative pose between the two objects, for example between an HMD (e.g., an HMD donned by the surgeon holding the tool) and the tool, can be determined. Typically, such adaptors are used to attach a tracking unit to an elongated instrument that is symmetric around its long axis, hence determining only 5-DOF of the pose (e.g., without the degree-of-freedom representing the rotation around this axis) is sufficient for generating guidance information for the surgeon (such adaptors can allow the surgeon, for example, to rotate the tracking unit towards tracking cameras, or to rotate the tracking unit so that it doesn't obscure the surgeon's view of the surgical field).

According to some embodiments, the fixing of tracking units 102a and 102b relative to first object 10a and third tracking unit 102c relative to second object 10b may be through direct attachment. According to some embodiments, the attachment is not permanent in the sense that tracking units 102 may be removed and later attached to other objects. According to some embodiments, tracking units 102a and 102b may be indirectly attached to first object 10a in the sense that the attachment of each is via a respective intermediate element (referred to as “adaptor” or “mount”; not shown). Similarly, according to some embodiments, third tracking unit 102c is indirectly attached to second object 10b. According to some embodiments, the locations and orientations in which the tracking units 102a and 102b are fixed to first object 10a may be predetermined (i.e., tracking units 102a and 102b may be fixed at designated locations and orientations on first object 10a, which are selectable). According to some embodiments, the relative poses between each of tracking units 102a and 102b and first object 10a may be determined following the fixing of tracking units 102a and 102b to first object 10a.

According to some embodiments, the attachment of tracking units 102a and 102b to first object 10a may be permanent. According to some such embodiments, tracking units 102a and 102b may be integrated into first object 10a in the sense of being fabricated together with first object 10a as a part thereof (e.g., permanent attachment).

According to some embodiments, any one of tracking units 102 can be fixed relative to any one of objects 10a and 10b. According to some such embodiments, each of tracking units 102 may be directly, indirectly, permanently, or temporarily (i.e. removably) attached to any one of objects 10a and 10b.

According to some embodiments, the term “relative pose” may be used in a broad sense to refer also to the three DOF representing the relative position of a second object with respect to a first object and two out of the three degrees of freedom representing the orientation of the second object relative to the first object. This may be particularly pertinent when the tracking unit is fixed onto an object which displays rotational symmetry about an axis thereof (e.g. a longitudinal axis thereof), using an adaptor that allows the tracking unit to swivel around the axis, so that specification of this last degree of freedom may be redundant.

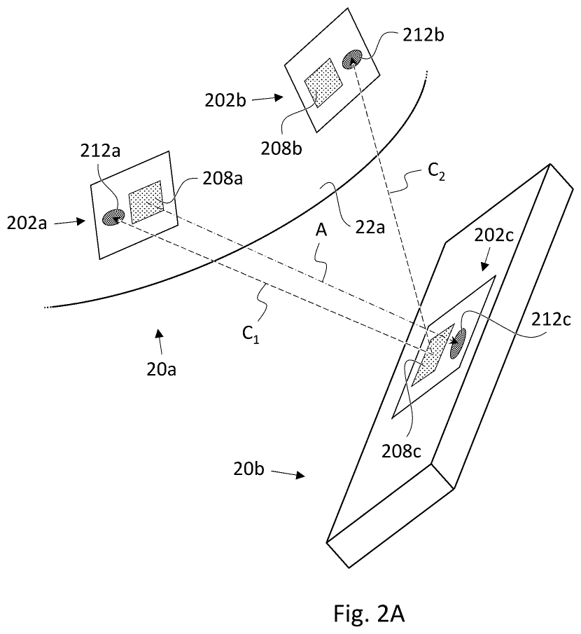

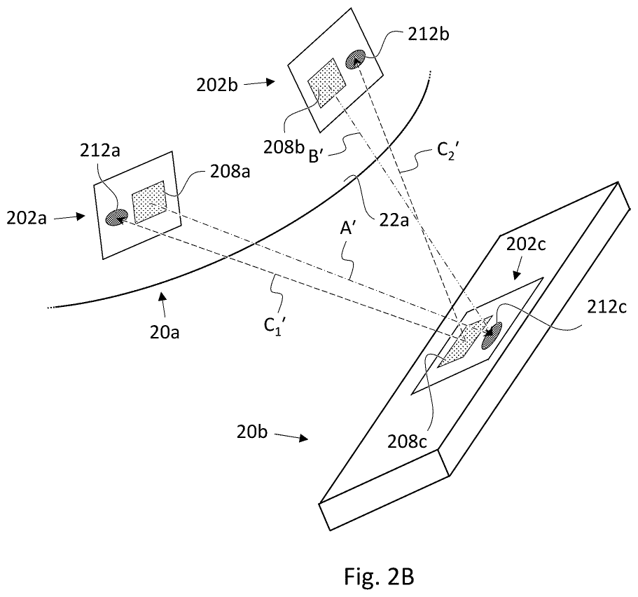

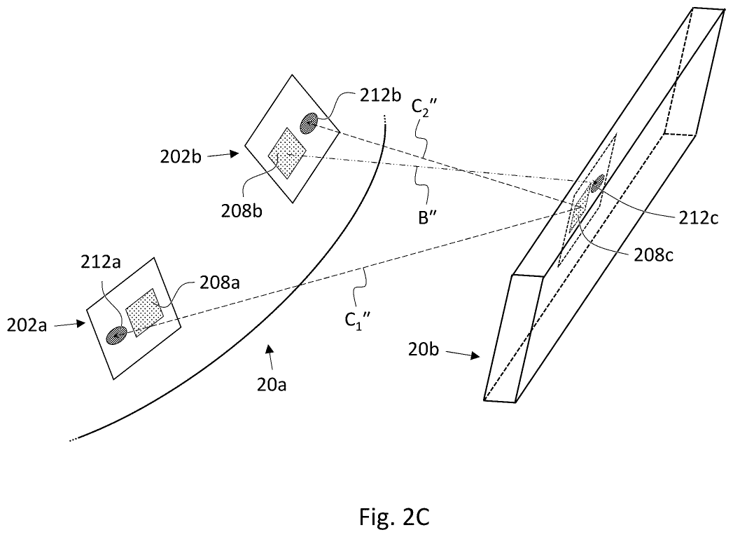

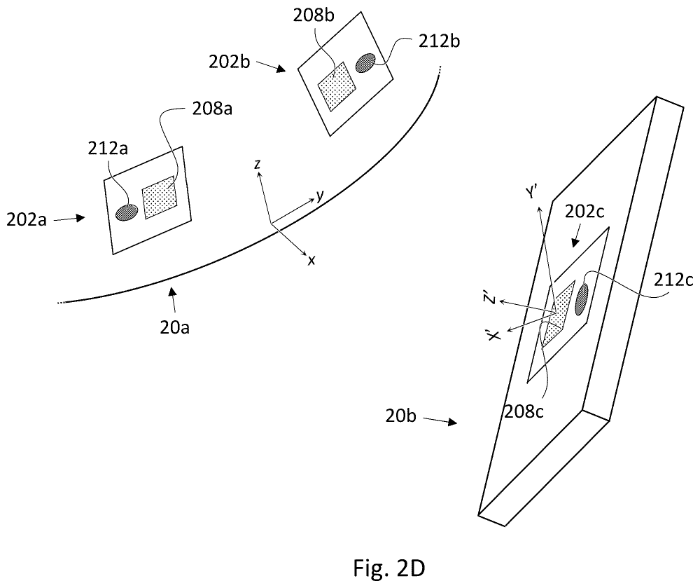

To facilitate the description, reference is made to FIGS. 2A-2D. Each of FIGS. 2A-2D depicts a first (optical) tracking unit 202a, a second (optical) tracking unit 202b, and a third (optical) tracking unit 202c, which correspond to specific embodiments of first tracking unit 102a, second tracking unit 102b, and third tracking unit 102c, respectively. First tracking unit 202a includes a first optical sensor 208a and a first visual indicator 212a, which correspond to specific embodiments of first optical sensor 108a and first visual indicator 112a, respectively. Second tracking unit 202b includes a second optical sensor 208b and a second visual indicator 212b, which correspond to specific embodiments of second optical sensor 108b and second visual indicator 112b, respectively. Third tracking unit 202c includes a third optical sensor 208c and a third visual indicator 212c, which correspond to specific embodiments of third optical sensor 108c and third visual indicator 112c, respectively. The graphical representation of optical sensors 208 is schematic without distinction between light sensitive elements and optical elements such as lenses, filters, and stops (e.g. aperture stops, field stops), which may also be included.

First tracking unit 202a and second tracking unit 202b are shown attached onto a first object 20a (partially shown). Third tracking unit 202c is shown attached onto a second object 20b. First object 20a and second object 20b correspond to specific embodiments of first object 10a and second object 10b, respectively.

In the configuration depicted in FIG. 2A first optical sensor 208a is employed to image third visual indicator 212c, and third optical sensor 208c is employed to image visual indicators 212a and 212b. In the configuration depicted in FIG. 2B first optical sensor 208a and second optical sensor 208b are both employed to image third visual indicator 212c, and third optical sensor 208c is employed to image visual indicators 212a and 212b. In the configuration depicted in FIG. 2C, second optical sensor 208b is employed to image third visual indicator 212c, and third optical sensor 208c is employed to image visual indicators 212a and 212b. In each of FIGS. 2A-C different optical sensors are employed, based on whether or not the visual indicator is in the FOV of the optical sensor, based on the relative pose between the objects, based on employment in previous tracking cycles, based on optimizing accuracy and/or obscurations (e.g., when more than one object is tracked relative to the first object, as will be described further below, each sensor can be employed in tracking the closest object), and/or based on additional considerations (e.g., computer hardware 104 may employ both first optical sensor 208a and second optical sensor 208b during a calibration process as described further below), or any combinations thereof. According to some embodiments, computer hardware 104 may be configured to select from first tracking unit 102a and second tracking unit 102b, whether both thereof, or which thereof, will be used to track third optical tracking unit 102c (e.g., to image visual indicator 212c). The selection may be updated in real-time based on the data acquired by optical sensors 108. It is noted that the field of illumination of the visual indication is typically bigger than the FOV of the sensor. Hence it is possible, for example, that visual indicator 212c is not in the FOV of optical sensor 208a, although visual indicator 212a is in the FOV of optical sensor 208c. This dynamic selection may advantageously enhance the range of tracking coverage and also facilitate uninterrupted tracking (an interruption can be caused, for example, by partial obstruction to the FOV of an optical sensor of one of the two tracking units 202a and 202b).

According to some embodiments, when the visual indicators are light emitting (e.g., when visual indicators are LEDs), they can be employed to emit pulses of light that are synchronized with the integration time of the opposite optical sensor (or sensors). Similarly, when the visual indicators are reflectors, the light that they reflect can be emitted in pulses that are synchronized with the integration time of the opposite optical sensors. For example, in the configuration depicted in FIG. 2A, pulses generated by third visual indicator 212c can be synchronized with the integration time of first optical sensor 208a, and pulses generated by visual indicators 212a and 212b can be synchronized with the integration time of third optical sensor 208c. In some of these embodiments, all employed LEDs and all employed optical sensors can be employed concurrently (e.g., all are synchronized to be employed simultaneously). In some of these embodiments, LEDs can be synchronized with opposite sensors, but not all LEDs are synchronized. For example, pulses generated by third visual indicator 212c can be synchronized with the integration time of first optical sensor 208a, and pulses generated by visual indicators 212a and 212b can be synchronized with the integration time of third optical sensor 208c, but the pulses generated by third visual indicator 212c are not synchronized with the pulses generated by visual indicators 212a and 212b. In some of these embodiments, pulses generated by visual indicators 212a and 212b are not synchronized with one another but are separately synchronized with integration times of third optical sensor 208c.

Referring again also to FIG. 1, computer hardware 104 includes one or more processors, and, optionally, RAM and/or non-volatile memory components (not shown). According to some embodiments, computer hardware 104 may additionally include a communication unit (not shown), being thereby configured to receive from optical tracking units 102 raw data acquired by each of optical sensors 108 (e.g., image data) and/or processed data based on the raw data (e.g., blob data and/or histogram data). According to some embodiments, the communication unit can be a wireless communication unit including, for example, a Wi-Fi module, a Bluetooth module, and/or a Bluetooth Low Energy (BLE) module (not shown).

According to some embodiments, all components of computer hardware 104 may be included within a single housing. According to some embodiments, computer hardware 104 may be included in a portable computing device. According to some alternative embodiments, components computer hardware 104 may be distributed. According to some such embodiments, the processing and control circuitries of tracking units 102 form part of computer hardware 104. According to some embodiments, computer hardware 104 may be a cloud computer. According to some embodiments, a processor may be comprised or embedded with one or more tracking unit(s). In some embodiments, one or more processors may be communicatively and/or physically associated with the tracking unit(s).

Computer hardware 104 is configured to determine a relative pose between first object 10a and second object 10b, based at least on:
A first data set indicative of two directions from third optical sensor 108c towards first visual indicator 112a and second visual indicator 112b, respectively.
A second data set indicative of a direction from first optical sensor 108a towards third visual indicator 112c and/or a direction from second optical sensor 108b towards third visual indicator 112c.
Information specifying, or indicative of, a relative pose between first tracking unit 102a and first object 10a, between second tracking unit 102b and first object 10a, and between third tracking unit 102c and second object 10b.

Here it is implicit that the following condition is met: The relative pose of first object 10a and second object 10b (and tracking units 102 fixed relative thereto, respectively) is such that third tracking unit 102c is capable of “seeing” (in the sense of having in the FOV thereof) each of first tracking unit 102a and second tracking unit 102b (or, more precisely, each of first and second visual indicators 112a and 112b) and simultaneously being seen by at least one of first tracking unit 102a and second tracking unit 102b (i.e. third visual indicator 112c is in the FOV of at least one of first optical sensor 108a and second optical sensor 108b). Referring again to FIGS. 2A-2C, stated with respect to first object 20a and second object 20b, and tracking units 202, the above condition implies that at least one of the three configurations depicted in FIGS. 2A-2C can be realized.

In some embodiments, tracking system 100 determines a relative pose between the coordinate system associated with the first and second tracking units and the coordinate system associated with the third tracking unit only when these conditions are met. In some embodiments, when one or more of these conditions are not met, tracking system 100 determines a partial pose (e.g., not the full 6-DOF) between said coordinate systems. In some embodiments, the relative pose between the first tracking unit and the second tracking unit is fully known (e.g., all 6 DOF are known), but the relative pose between the coordinate system associated with the first and second tracking units and the first object 10a is only partially known (e.g., only 5 DOF are known). Similarly, in some embodiments, the relative pose between the third tracking unit and the second object 10b is only partially known (e.g., as in the example of a tracking unit fixed to a tool with an adaptor that allows the tracking unit to swivel around the tool). In both cases, when the above conditions are met, tracking system 100 determines a full pose between the coordinate system associated with the first and second tracking units and the coordinate system associated with the third tracking unit, and only a partial pose between the objects.

In some embodiments, as detailed herein, the relative pose between the first tracking unit and the second tracking unit is required in order to calculate the pose between a joint coordinate system associated with said two tracking units and a coordinate system associated with said third tracking unit. The relative pose between first tracking unit and second tracking unit can be derived from the two poses of each of the first and second tracking units relative to the joint coordinate system (e.g. a coordinate system of an object), or can be directly determined as described herein below (e.g., without necessarily determining their poses relative to an object). In this case, for example, the coordinate system of the first tracking unit can be used as the joint coordinate system for both the first and second tracking units, without the need for a coordinate system of an object. Nevertheless, even though objects are not essential for determining the relative pose between the tracking units, typically the tracking system disclosed is employed to track objects, and the objects have their own coordinate systems. In such cases, the known poses between the tracking units and the respective objects can be used to derive the pose between objects.

As used herein, by a data set “indicative of a direction” what is meant is a data set that explicitly specifies the direction (e.g. specifies the azimuth and the elevation, or the yaw and the pitch) or at least includes data (e.g. blob data or raw image data from which blob data can be extracted, when the optical sensor is a pixel-array sensor, or an analog output when the optical sensor is an analog position sensing detector) from which the direction may be determined by computer hardware 104 (e.g., based on a previously determined mapping between 2D locations in a sensor and directions). Thus, according to some embodiments, computer hardware 104 may be further configured to determine, based on data (e.g. blob data) acquired by an optical sensor (such as any one of optical sensors 108), at least one direction from the optical sensor towards at least one visual indicator, which is in the FOV of the optical sensor.

More specifically, by way of a non-limiting example, according to some embodiments of system 100 including tracking units 202, per the configuration depicted in FIG. 2A, based on data acquired by optical sensors 208a and 208c, computer hardware 104 is configured to compute: (a) the direction (indicated by a dashed-dotted arrow A) from first optical sensor 208a to third visual indicator 212c, (b) the direction (indicated by a first dashed arrow C₁) from third optical sensor 208c to first visual indicator 212a, and (c) the direction (indicated by a second dashed arrow C₂) from third optical sensor 208c to second visual indicator 212b.

Per the configuration depicted in FIG. 2B, based on data acquired by optical sensors 208a, 208b, and 208c, computer hardware 104 may compute: (a′) the direction (indicated by a dashed-dotted arrow A′) from first optical sensor 208a to third visual indicator 212c, (b′) the direction (indicated by a dashed-double-dotted arrow B′) from second optical sensor 208b to third visual indicator 212c, (c′) the direction (indicated by a first dashed arrow C₁′) from third optical sensor 208c to first visual indicator 212a, and (d′) the direction (indicated by a second dashed arrow C₂′) from third optical sensor 208c to second visual indicator 212b.

Per the configuration depicted in FIG. 2C, computer hardware 104 may compute: (a″) the direction (indicated by a dashed-double-dotted arrow B″) from second optical sensor 208b to third visual indicator 212c, (b″) the direction (indicated by a first dashed arrow C₁″) from third optical sensor 208c to first visual indicator 212a, (c″) the direction (indicated by a second dashed arrow C₂″) from third optical sensor 208c to second visual indicator 212b.

According to some embodiments, computer hardware 104 is configured to search for the relative pose between first object 10a and second object 10b, which is consistent with the computed directions, or, more generally, the measured data (e.g. blob data), the relative poses between first object 10a and each of tracking units 102a and 102b, and the relative pose between second object 10b and tracking unit 102c.

According to some embodiments, computer hardware 104 may be configured to directly determine (e.g. using a neural network) the relative pose between first object 10a and second object 10b from raw data or blob data, i.e. without intermediate determination of the two directions from third optical sensor 108c towards visual indicators 112a and 112b and the direction from first optical sensor 108a towards third visual indicator 112c and/or the direction from second optical sensor 108b towards third visual indicator 112c.

Referring again to FIGS. 2A-2C, first tracking unit 202a and second tracking unit 202b are fixed relative to first object 20a, and relative to a first coordinate system in which each of first tracking unit 202a, second tracking unit 202b, and first object 20a is stationary. Similarly, third tracking unit 202c is fixed relative to second object 20b, and relative to a second coordinate system in which each of third tracking unit 202c and second object 20b is stationary. Reference is made to FIG. 2D, showing a first coordinate system (x, y, z) that is associated with (e.g., attached to) first object 20a; a second coordinate system (x′, y′, z′) that is associated with (e.g., attached to) second object 20b. The skilled person will readily appreciate that the depicted choices of the origins of the coordinate systems are arbitrary and other choices equally apply.

According to some embodiments, the calculation of the relative pose between two tracking units 102a and 102b and third tracking unit 102c (e.g., between the joint coordinate system for both the first and second tracking units and the coordinate system of the third tracking unit), using the three (or four) detected directions from the optical sensors to the opposite visual indicators, is based on a known relative pose between first tracking unit 102a and second tracking unit 102b. According to these embodiments, based on this relative pose, and based on the known location of each visual indicator relative to its corresponding optical sensor, computer hardware 104 can derive the location of visual indicator 112b relative to optical sensor 108a and the location of visual indicator 112a relative to optical sensor 108b, and consequently find the relative pose between the joint coordinate system for both the first and second tracking units and the coordinate system of the third tracking unit, which is consistent with the three (or four) detected directions.

According to some embodiments, computer hardware 104 may be further configured to determine the relative pose between first tracking unit 102a and second tracking unit 102b. According to some embodiments, the determination of the pose between tracking units 102a and 102b may be performed by another processing unit, in the factory or in the field. According to some embodiments, in both cases the determination can be performed as detailed herein below.

According to some embodiments, the relative pose between first tracking unit 102a and second tracking unit 102b may be determined by employing third tracking unit 102c. To this aim, multiple sets of directions (or data indicative thereof) are acquired (e.g., sampled) comprising two directions from third optical sensor 108c towards first visual indicator 112a and second visual indicator 112b, respectively, a direction from first optical sensor 108a towards third visual indicator 112c, and a direction from second optical sensor 108b towards third visual indicator 112c. Each of the acquired sets includes the directions (or data indicative thereof) obtained at one of a plurality of relative poses between first object 10a and second object 10b. Based on an initial guess for the relative pose between first tracking unit 102a and second tracking unit 102b, for each of the acquired sets of directions, two relative poses (e.g., each pose consisting of 6-DOF) can be derived: a relative pose between the coordinate system of first tracking unit 102a and the coordinate system of third tracking unit 102c, and a relative pose between the coordinate system of second tracking unit 102b and the coordinate system of third tracking unit 102c. Since first tracking unit 102a and second tracking unit 102b are fixed to one another, when the correct relative pose between first tracking unit 102a and second tracking unit 102b is used for the calculation, the difference between the two relative poses should be constant for all of the acquired sets of directions. To this end, according to some embodiments, computer hardware 104 executes an iterative optimization algorithm, which is configured to receive as inputs the acquired data sets and output the relative pose between first tracking unit 102a and second tracking unit 102b.

According to other embodiments, the relative pose between first tracking unit 102a and second tracking unit 102b may be determined by employing a second set of two tracking units that are pre-calibrated (e.g., the relative pose between these two tracking units of the second set was previously determined). To this aim, pairs of poses (e.g., each pair consisting of two 6-DOF values) are sampled between first tracking unit 102a (e.g., using only the sensor and visual indicator of first tracking unit 102a, without the visual indicator of second tracking unit 102b) and the second set of tracking units, and between second tracking unit 102b (using only the sensor and visual indicator of second tracking unit 102b) and the second set of tracking units. The relative pose between first tracking unit 102a and second tracking unit 102b can thus be derived from each such pair of poses (e.g., by calculating the difference between the two poses), and can be averaged over these pairs.

By using any of the above methods, after two (or more) optical tracking units are fixed to a first object, the relative pose between them can be readily determined. In the embodiments where the two (or more) tracking units have at least a partially overlapping FOV, this can be based on sampled data sets that are acquired while moving and rotating a second object within this overlapping FOV, the second object comprising one or more tracking units. The second object can be, for example, a hand-held object. Typically, a relative pose between at least one of the tracking units that are fixed to the first object and the coordinate system of the first object is also determined. As described further below, when the first object is a vehicle, this can be, for example, based on a cross displayed via a head-up display (in this case, for example, the second object can comprise a camera and a tracking unit, the camera being tracked while imaging the head-up display), or based on an accurate mounting of an additional tracking unit on a mechanical reference. In another example, when the first object is a surgical exoscope, this can be done, for instance, by using a second object comprising a visible calibration target (e.g., a target that can be imaged by the exoscope cameras) at a known location relative to the tracking unit (or units) of the second object. To allow the calibration, the visible target can be imaged by the exoscope cameras simultaneously with the sampling of the tracker data sets.

According to some embodiments, the mounting mechanism between the tracking unit (or units) and the object supports an accurate (e.g. repeatable to a desired degree) mounting. According to some of these embodiments, the pose of the mounting component of each of the tracking units relative to the tracking unit's coordinate system can be determined during manufacturing (e.g., by calibration). Similarly, the pose of each mounting site in the object can be determined relative to the object's coordinate system (for example, by calibration during manufacturing). Thereafter, each of the tracking units can be attached to any of the respective mounting sites, and the relative pose between the tracking unit's coordinate system and the object's coordinate system can be derived based on the pre-determined (e.g., pre-calibrated) poses. For example, the mounting component may be based on a dovetail locking mechanism, a kinematic mount, a Picatinny mount, and the like. In some embodiments, the mounting mechanism supports an accurate mounting even when the object is covered with a sterile drape. For example, a surgical exoscope may be draped, and single-use (e.g. disposable), sterile tracker units may be fixed to the exoscope after it is draped, such that the mounting sites (e.g., which are part of the object) are under the drape, and the tracking units are above the drape. In some embodiments, multiple mounting sites on the object are pre-calibrated (e.g., their poses relative to the object's coordinate system are predetermined). The mounting sites can then be used for fixing two (or more) tracking units to any pair (or more) of the multiple mounting sites, and the relative poses between the tracking units and the object can be derived as described above. This can allow a flexibility of selecting the mounting sites to be utilized based on the need. For example, two tracking units can be fixed to an exoscope such that a line-of-sight between the tracking units and a third tracking unit (e.g., a third tracking unit that is fixed relative to a patient) can be kept unobstructed throughout a surgical procedure that requires a specific location of the exoscope relative to the patient. In a different procedure the exoscope may be positioned differently relative to the patient, and other mounting sites can be utilized.

According to some alternative embodiments, the relative pose between the first tracking unit 102a and second tracking unit 102b may be directly derived, for example, based on a known mechanical design of the first object and/or a known mechanical design of the tracking units. It is noted that pre-calibrated mounting sites and/or the use of a known mechanical design to derive a relative pose between a tracking unit and an object can be also used with objects that are designed to be fixed with a single tracking unit.

According to some embodiments, first tracking unit 102a and second tracking unit 102b may be mounted, or removably mountable, on a same frame (not shown in the Figures), which is configured to be attached to an object, such as first object 10a. The mounting may be such that first tracking unit 102a and second tracking unit 102b are fixed relative to one another and the relative pose therebetween is known or pre-calibrated.

It is noted that other methods known in the art can be used for determining the relative pose between the first tracking unit 102a and second tracking unit 102b and/or the relative pose between any of the first tracking unit 102a and second tracking unit 102b and the object, both for field calibration and for factory calibration.

Referring again to FIGS. 2A-2D, first object 20a is shown as having a curved surface 22a (on which both of first tracking unit 202a and second tracking unit 202b are mounted), and second object 20b is shown as box-shaped. However, the skilled person will understand these shapes to be no more than non-limiting examples and that other shapes are equally applicable. Generally, any shapes of (a pair of) objects, onto which tracking units 202 may be attached, are applicable so long as the shapes do not preclude use of tracking units 202 to determine the relative pose between the objects when the objects are positioned and oriented in accordance with intended use thereof.

According to some embodiments, first object 10a may be a head mounted display (HMD). According to some such embodiments, wherein system 100 is configured to be used in surgery, second object 10b may be, for example, a surgical hand-held tool. Typically, large surgical devices may be fitted with two or more tracking units (e.g. tracking units 102a and 102b), while small instruments and tools (e.g. handheld tools) may be fitted with a single tracking unit (e.g. third tracking unit 102c). Examples for large devices are a surgical microscope, an exoscope, a surgical robot, a robotic arm, an imaging device (such as an intraoperative CT or a C-arm), and a surgical light. Examples for hand-held instruments or tools are a tap, a drill, a laparoscope, and an ultrasound transducer.

It is noted that in embodiments where first object 10a is a binocular HMD with an interpupillary distance (IPD) mechanism (e.g., a mechanism for adjusting the distance between the left and right displays, so it can be set to fit the user's IPD), there are two possible options for fixing (e.g., embedding or attaching) tracking units 102a and 102b to the HMD. In the first option tracking units 102a and 102b are fixed relative to each other, and do not move when the IPD mechanism is employed. In this case, the poses between the coordinate systems of tracking units 102a and 102b and the coordinate systems of the two displays (e.g., the displays for the left and right eyes of the user) can be calibrated during manufacturing. These poses can be essential when the HMD is used in VGS applications where virtual objects displayed by the HMD are expected to be accurately aligned with a patient's anatomy. An accurate IPD mechanism can guarantee a repeatable and/or known pose between the coordinate systems of the two displays and the coordinate systems of the two tracking units for different IPD values (e.g., it can be calibrated for multiple IPD values). In the second option each of tracking units 102a and 102b is fixed relative to one of the two displays. In this option the pose between each tracking unit and its corresponding display is calibrated during manufacturing. The IPD mechanism in this option is not required to be accurate, since the relative pose between the two tracking units can be easily determined every time the IPD is adjusted, by using any of the methods described above (e.g., by employing a third tracking unit, by employing a second, pre-calibrated set of two tracking units, or by employing a third tracking unit comprising two distant visual indicators). In both the above options, a quick calibration can be performed by the user to determine the exact position of each of the user's eyes relative to its corresponding display. Alternatively, an eye tracker can be employed. These positions can be essential for accurate augmentation on a patient's anatomy in VGS applications. These positions can also be essential in embodiments where distortions associated with the HMD optics (e.g., distortions in the image displayed via the HMD and/or distortions of the real world as seen through the semi-transparent display optics) are more sensitive to the position of the eye relative to the display. Distortions in visor projection HMDs, for example, are more sensitive to the position of the eye relative to the display, and distortions in waveguide-based HMDs, for example, are less sensitive.

It is noted that the total number of tracking units that are available for implementing tracking between two objects can be bigger than three (e.g., more than one tracker unit fixed to one object and/or more than two tracking units fixed to a second object). In some embodiments, computer 104 is configured to select which tracking units to employ for tracking. For instance, computer 104 can employ two units from the first object and one unit from the second object, or vice versa. The selection of which tracking units to employ can be dynamic and can be based on various criteria.

According to some embodiments, not depicted in FIG. 1, third tracking unit 102c (or at least the functional part thereof, when it comprises two sub-assemblies) may be fixed relative to a subject (e.g. a patient undergoing surgery). For example, third tracking unit 102c may be configured to be mounted on an adaptor, such as a Mayfield clamp, in embodiments wherein system 100 is used during brain surgery, or a clamp, a screw, or a pin configured to be attached to a bone of a subject in embodiments wherein system 100 is used during an orthopedic (such as spine) surgery (e.g., a clamp configured to be attached to a spinous process during spine surgery). Third tracking unit 102c may also be configured to be directly mounted on the skin of a subject, for instance with an adhesive tape. The use of system 100 in surgery is described in detail below.

Following the fixing of a tracking unit to a subject, the tracking unit may be “registered” relative to the subject anatomy (i.e. the relative pose, or the registration, between the tracking unit and the subject anatomy may be determined). Similarly, following the fitting of a tracking unit to surgical tool, the tracking unit may be “aligned” relative to the tool (i.e. the relative pose, or the alignment, between the tracking unit and the tool may be determined). According to some embodiments, wherein a tracking unit is integrated into an object, the alignment may be performed during manufacturing of the object.

According to some embodiments, an object (e.g. first object 10a) may include one or more mounts, which are integrated thereinto. Each of the one or more mounts is configured to have fixed thereon a tracking unit at a respective single and repeatable pose relative to the object. The relative pose between the object and a tracking unit, which is attached to a mount, may be prespecified or measured following attachment of the tracking unit.

According to some embodiments, tracking units 102 may be fabricated to be similar with respect to one or more of size, shape, type of optical sensor, type of visual indicator, design of printed circuit boards, processing unit(s), communication unit(s), memory (or memories), arrangement of components therein, processing algorithm(s), communication protocol, and the like.

According to some embodiments, tracking units 102 are modular. As used herein, the term “modular” in reference to two or more devices/components is to be interpreted in an expansive manner in the sense of referring not only to identical design and/or function, but also similar design and/or function, and more generally any collection of devices/components, which may fulfill the same function and may be interchangeably employed. The modularity of tracking units 102 should be understood in this sense.

According to some embodiments, tracking units 102 are modular in the sense of being fabricated to the same design, or a similar design, or at least in being configured to function similarly. According to some embodiments, tracking units 102 may share a same or similar function (e.g. image a visual indicator in the FOV of the optical sensor thereof and generate data, such as blob data), a same or similar optical sensor, a same or similar processing unit(s), a same or similar communication protocol, a same or similar memory (e.g. in which the calibration data is stored), and/or the like. According to some embodiments, tracking units 102 are modular in the sense of being all configured to sense/detect and illuminate (or reflect) light having the same wavelength (e.g., same functionality). In some embodiments, when multiple tracking units are attached to multiple objects, once the computer hardware 104 receives the system “configuration” (e.g. which tracking unit/s is/are attached to which object, the alignment (pose) between each tracking unit and its respective object, etc.), it can employ all the tracking units for tracking objects relative to one another.

According to some embodiments, tracking units 102 have the same functionality but can differ, for instance, with respect to size, shape, and/or types of components. For example, a tracking unit configured to be fixed to a patient may comprise a larger battery than a tracking unit configured to be fixed to a tool, and an HMD tracking unit may not require a battery at all, as it is powered by the HMD. As another example, a tracking unit configured to be fixed to a patient and a tracking unit configured to be fixed to a tool may have different mounting mechanisms, for instance so that a user will not accidently attach a tool tracking unit to a patient and vice versa. As yet another example, different tracking units may have different fields of view (e.g., the FOVs of their optical sensors may differ), for instance because a tracking unit fixed to (or embedded in) an exoscope and a tracking unit attached to a tool may require different FOVs.

In accordance with some embodiments, the tracker system disclosed herein includes optical tracker units which have small scale form, and can be used for tracking a variety of objects, of different sizes and shapes, by attaching/fixing two or more tracking units relative to a first object and additional tracker units to other objects. As further detailed herein, in the advantageous inside-out/outside-in modular optical tracking system of the current disclosure, individual tracking units are separately attached to an object, thereby not only solving the need for a unique design of trackers per object, but also allowing to overcome space limitations imposed by the size and/or structure of an object. Thus, instead of associating the object with a large tracker unit (having two visual indicators), at least two relatively small, distinct tracker units, each including at least one visual indicator, can be more easily fixed to (or embedded in) the object. Thus, according to some embodiments of the current disclosure, the optical tracking system is modular. In particular, the tracking units may be same or similar with respect to size, shape, and/or structure, and not necessarily custom designed or tailored for attaching to a specific object, thereby advantageously allowing use of the same tracking units with a variety of objects of different size, structure, and/or function. Additional advantages associated with modularity include cost effectiveness.

For example, in a VGS system, as detailed herein below, two modular tracking units may be fixed to the HMD (first object) at a distance of, for example, 10-14 cm therebetween, and additional individual modular tracking units may be fixed to other objects and/or a subject (for example, a patient, surgical tools, etc.), all of which are typically at a distance of, for example, 30-70 cm from the HMD during surgery (e.g., when the surgeon is donning the HMD).

Another advantage of a modular system as disclosed herein, is enhancing the tracker robustness to line-of-sight obstruction by adding/fixing one or more additional tracking units relative to a tracked object. For example, three tracking units can be attached to an object (e.g., instead of only two). When a line-of-sight between one of the three tracking units and a tracking unit attached to a second object is blocked, the other two tracking units can be employed for tracking (e.g., redundancy improves robustness). In some embodiments, enhancing the tracker robustness to line-of-sight obstruction can be facilitated by attaching two (or more) tracking units to each of the two objects, and employing tracking units based on line-of-sight availability. For example, as detailed herein below, in an exemplary VGS system, two or more tracking units may be fixed to each of the HMD, a robotic arm, and an exoscope camera unit, such that the tracking between selected pairs of objects is more robust.

According to some embodiments, additional advantages of the modular optical trackers include enhancing tracker coverage by distributing tracking units around a tracked object, such as, for example, for tracking a user's HMD in a cockpit, in an armored vehicle, in a tank, and the like. In such instances, a plurality of modular optical tracker units can be installed/fixed around the user, and the dynamic selection of tracking units to be utilized for tracking (e.g., selection from the plurality of tracking units) can be based on the momentary HMD gaze/view direction. In this example, when, for instance, a single tracking unit is fixed to the HMD, two of the surrounding tracking units that are currently within the FOV of the HMD tracking unit can be employed. According to these embodiments, after installing the tracking units (e.g., in the cockpit/vehicle), the relative pose between pairs of tracking units (e.g., pairs of tracking units selected from the plurality of installed tracking units) can be known, for example, based on determining the relative pose between each of the tracking units and the aircraft/vehicle. Alternatively, the relative pose between some of the tracking units and the aircraft/vehicle can be determined, and the relative pose between these tracking units and the other tracking units (e.g., other tracking units selected from the plurality of installed tracking units) can be determined directly. Determining the pose between an installed tracking unit and the vehicle (“object”) coordinate system can be done based on any reference coordinate system available in the vehicle, such as, for example, a cross displayed via an aircraft head-up display that can be imaged by a tracked camera, a mechanical reference that can be used to mount a tracking unit at a known pose, and the like.

According to some embodiments, the tracking unit has wireless communication capabilities. According to some embodiments, the tracking unit is powered by a battery. Each of these embodiments further simplify the attachment of a tracker unit to an object (e.g., both in case of a permanent installation/fixation to an object, and in case of a temporary attachment/fixation relative to an object).

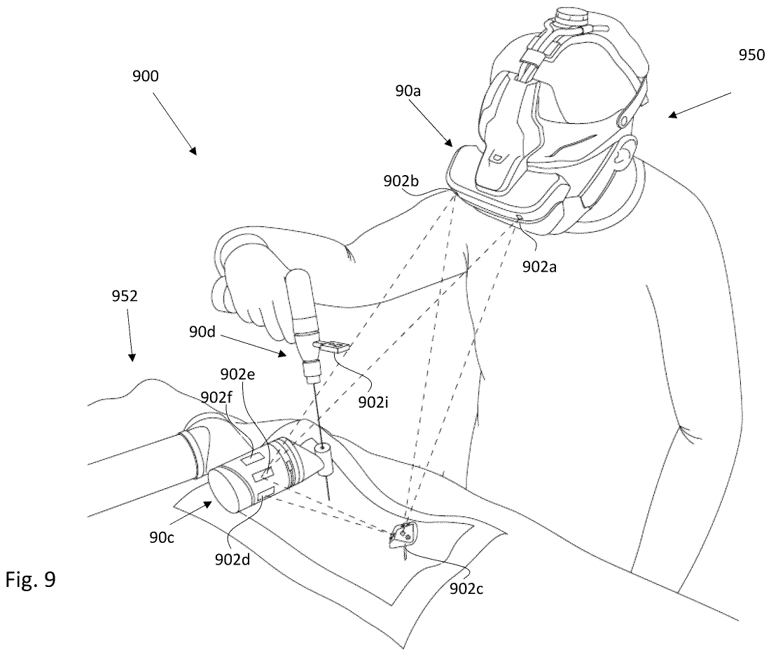

According to some embodiments, (as depicted, for example, in FIG. 9), even though only one optical sensor and one visual indicator per tracking unit suffice for tracking, each tracking unit may include two or more optical sensors and/or two or more visual indicators, without compromising the small form factor of the tracking unit. A plurality of optical sensors and/or visual indicators in a single tracking unit can be useful, for example, when the tracking unit is employed in tracking a relative pose with respect to more than one other object. As another example, a plurality of optical sensors and/or visual indicators in a single tracking unit can be useful to enhance the FOV of the tracking unit by having different optical sensors facing different directions (e.g., with partial overlap between the fields of view of two sensors facing adjacent directions). A plurality of optical sensors and/or visual indicators can also provide redundancy. Using the modular optical tracking units disclosed herein, there is no restriction on the minimal distances between two visual indicators in a tracking unit, since utilizing a single visual indicator in each tracking unit is sufficient for determining a relative pose between two objects. Similarly, there is no restriction on a distance between the two or more optical sensors, since accurate tracking can be achieved by utilizing a single optical sensor in each tracking unit. In particular, even when a tracking unit includes two or more optical sensors and/or two or more visual indicators, a small form factor may be maintained.

According to some embodiments, when the visual indicator is an LED, the tracking unit may include, for example, two LEDs, even though only one of the LEDs is employed at a time for determining the relative pose. While both LEDs may be in principle employed simultaneously, typically, due to the small size of the tracking unit (and hence the short distance between the LEDs), employing more than one LED (at a time) can be non-advantageous with respect to accuracy. A tracking unit with two LEDs can be advantageous, for example with respect to redundancy. For instance, when a blood droplet occludes one of the LEDs, the second LED can be employed instead.

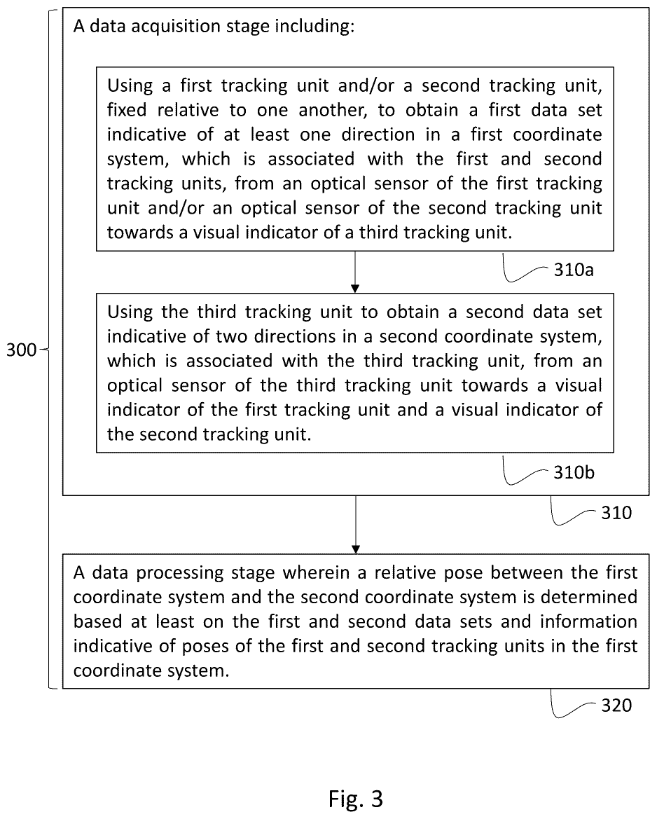

According to an aspect of some embodiments, there is provided an optical tracking-based method for determining a relative pose between a first coordinate system and a second coordinate system. FIG. 3 presents a flowchart of such a method 300, according to some embodiments. Method 300 may be implemented using an optical tracking system, such system 100. According to some embodiments, method 300 includes:
A data acquisition stage 310 including:
   A substage 310a, wherein a first tracking unit (e.g. first tracking unit 202a) and/or a second tracking unit (e.g. second tracking unit 202b) are used to obtain a first data set indicative of at least one direction in a first coordinate system, which is associated with the first and second tracking units, from an optical sensor of the first tracking unit and/or an optical sensor of the second tracking unit towards a visual indicator of a third tracking unit (e.g. third tracking unit 202c). The first and second tracking units are fixed relative to one another.
   A substage 310b, wherein the third tracking unit is used to obtain a second data set indicative of two directions in a second coordinate system, which is associated with the third tracking unit, from an optical sensor of the third tracking unit towards a visual indicator of the first tracking unit and a visual indicator of the second tracking unit.
A data processing stage 320, wherein a relative pose between the first coordinate system and the second coordinate system is determined based at least on the first and second data sets and information indicative of poses of the first and second tracking units in the first coordinate system.

some embodiments, wherein method 300 is used to provide real-time tracking, substages 310a and 310b are performed substantially simultaneously (e.g., overlapping sequence).

According to some embodiments, method 300 may further include an initial stage wherein the first and second tracking units are fixed relative to one another.

According to some embodiments, the first and second tracking units may be fixed relative to one another by fixing each to a first object (e.g. first object 20a). According to some embodiments, the third tracking unit may be fixed relative to a second object (e.g. second object 20b).

Data processing stage 320 may be implemented as described above in the description of the operation of computer hardware 104. In particular, data processing stage 320 may be implemented using computer hardware, such as computer hardware 104.

According to some embodiments, data processing stage 320 may additionally include initial processing substages, implemented prior to the determination of the relative pose between the first coordinate system and the second coordinate system. The initial processing substages may include:
A first initial processing substage, wherein (if obtained) the tracking data obtained by the first tracking unit, is used to determine a direction (e.g. the direction indicated by arrow A′ in FIG. 2B) from an optical sensor thereof (e.g. first optical sensor 208a) to a visual indicator (e.g. third visual indicator 212c) of the third tracking unit, and/or wherein (if obtained) the tracking data obtained by the second tracking unit is used to determine a direction (e.g. the direction indicated in by arrow B′ in FIG. 2B) from an optical sensor thereof (e.g. second optical sensor 208b) to the visual indicator of the third tracking unit.
A second initial processing substage, wherein the tracking data obtained by the third tracking unit is used to determine two directions (e.g. the directions indicated by arrows C₁′ and C₂′ in FIG. 2B) from an optical sensor thereof (e.g. third optical sensor 208c) to a visual indicator (e.g. first visual indicator 212a) of the first tracking unit and a visual indicator (e.g. second visual indicator 212b) of the second tracking unit, respectively.

According to some such embodiments, wherein the first and second tracking units are fixed relative to a first object and the third tracking unit is fixed relative to a second object, and the relative pose between the first object and the second object is to be determined, the relative pose therebetween is computed at least based on (i) the directions determined in the first initial processing substage and the second initial processing substage, and (ii) information indicative of the relative poses between the first object and each of the first and second tracking units, and of the relative pose between the second object and the third tracking unit, as described above in the description of system 100. Here it is implicit that the internal structure of each of the tracking units is known (e.g., that the location of each visual indication relative to its corresponding optical sensor is known).

Generally, the skilled person will readily perceive that system 100 may be used to determine the relative pose between a first coordinate system—defined by first tracking unit 102a and second tracking unit 102b and in which both are stationary (i.e. a coordinate system attached thereto)—and a second coordinate system defined by (i.e. attached to, or associated with) third tracking unit 102c. In particular, when each of first tracking unit 102a and second tracking unit 102b is fixed relative to an object (e.g. first object 10a), then the first coordinate system is not only attached to tracking units 102a and 102b but also to the first object. Similarly, when third tracking unit 102c is attached to a second object (e.g. second object 10b), then the second coordinate system is not only attached to third tracking unit 102c but also to the second object. Thus, when first and second tracking units 102a and 102b are fixed relative to a first object (e.g. first object 10a) and third tracking unit 102c is fixed relative to a second object (e.g. second object 10b), then specification of the relative pose between the first and second coordinate systems (together with specification of the poses of the first object in the first coordinate system and the second object in the second coordinate system) allows determining the relative pose between the first and second objects. Similarly, the skilled person will readily perceive that method 300 may be used to the same end.

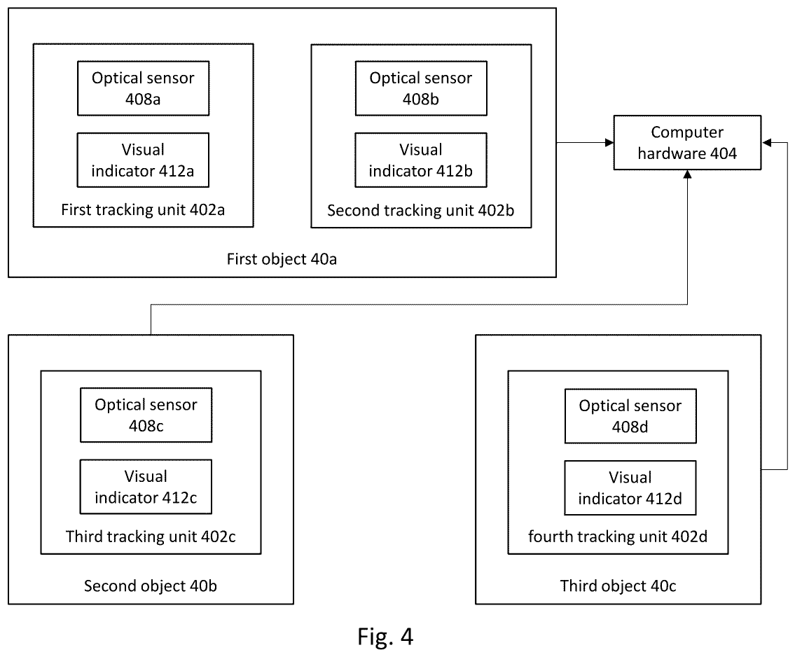

According to an aspect of some embodiments, there is provided an optical tracking system for determining relative poses between pairs of objects selected from three objects. FIG. 4 presents a block diagram of such an optical tracking system 400, according to some embodiments. System 400 includes four (optical) tracking units 402 (“tracking units 402” for short): a first tracking unit 402a, a second tracking unit 402b, a third tracking unit 402c and a fourth tracking unit 402d. Tracking units 402 may be similar to tracking units 102. In particular, first and second tracking units 402a and 402b may be similar to first and second tracking units 102a and 102b, while each of third and fourth tracking units 402c and 402d may be similar to third tracking unit 102c. System 400 further includes computer hardware 404 communicatively associated with tracking units 402. Computer hardware 404 may be similar to computer hardware 104.

Tracking units 402 include four optical sensors 408 and four visual indicators 412, respectively: First tracking unit 402a includes a first optical sensor 408a and a first visual indicator 412a, second tracking unit 402b includes a second optical sensor 408b and a second visual indicator 412b, third tracking unit 402c includes a third optical sensor 408c and a third visual indicator 412c, and fourth tracking unit 402d includes a fourth optical sensor 408d and a fourth visual indicator 412d.

The skilled person will readily perceive that in essentially the same manner as described above with respect to first object 10a, second object 10b, and system 100, computer hardware 404 may be configured to determine: (i) the relative pose between first object 40a and second object 40b, based on data acquired by first and third tracking units 402a and 402c, and (ii) between first object 40a and third object 40c, based on data acquired by second and fourth tracking units 402b and 402d, or vice-versa in the sense that the roles of first tracking unit 402a and second tracking unit 402b are reversed (i.e. data acquired by second tracking unit 402b is used in determining the relative pose between first object 40a and second object 40b and data acquired by first tracking unit 402a is used in determining the relative pose between first object 40a and third object 40c). The relative pose between second object 40b and third object 40c may be determined based on the computed relative poses between first object 40a and each of second object 40b and third object 40c.

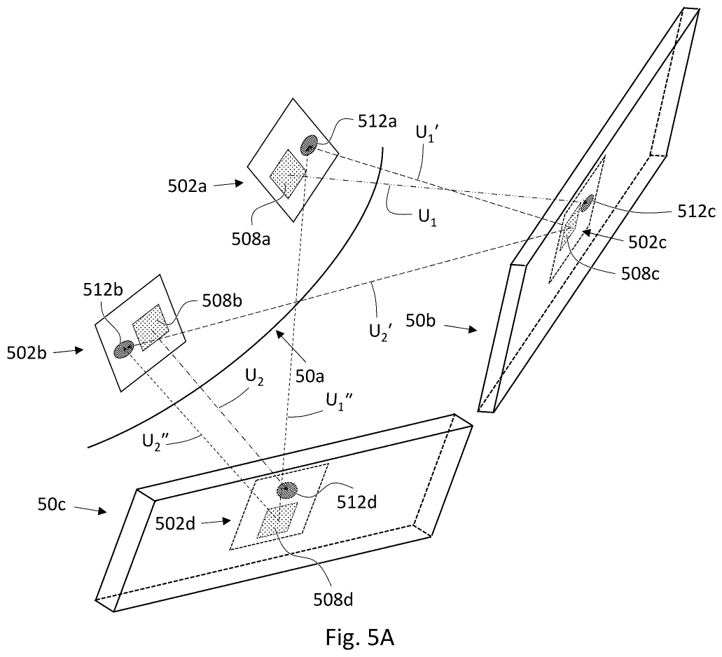

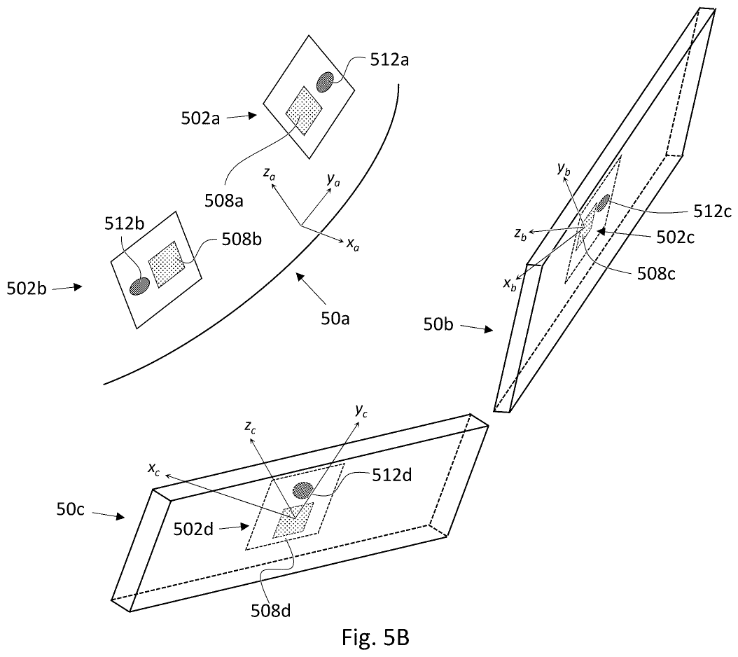

To facilitate the description, reference is made to FIGS. 5A and 5B. Each of FIGS. 5A and 5B depicts a first (optical) tracking unit 502a, a second (optical) tracking unit 502b, a third (optical) tracking unit 502c, and a fourth (optical) tracking unit 502d, which correspond to specific embodiments of first tracking unit 402a, second tracking unit 402b, third tracking unit 402c, and fourth tracking unit 402d, respectively. First tracking unit 502a includes a first optical sensor 508a and a first visual indicator 512a, which correspond to specific embodiments of first optical sensor 408a and first visual indicator 412a, respectively. Second tracking unit 502b includes a second optical sensor 508b and a second visual indicator 512b, which correspond to specific embodiments of second optical sensor 408b and second visual indicator 412b, respectively. Third tracking unit 502c includes a third optical sensor 508c and a third visual indicator 512c, which correspond to specific embodiments of third optical sensor 508c and third visual indicator 512c, respectively. Fourth tracking unit 502d includes a fourth optical sensor 508d and a fourth visual indicator 512d, which correspond to specific embodiments of fourth optical sensor 508d and fourth visual indicator 512d, respectively. The graphical representation of optical sensors 508 is schematic without distinction between light sensitive elements and optical elements such as lenses, filters, and stops (e.g., aperture stops, field stops), which may also be included.

First tracking unit 502a and second tracking unit 502b are shown attached onto a first object 50a (partially shown). Third tracking unit 502c is shown attached onto a second object 50b. Fourth tracking unit 502d is shown attached onto a third object 50c. First object 50a, second object 50b, and third object 50c, correspond to specific embodiments of first object 40a, second object 40b, and third object 40c, respectively.

In the configuration depicted in FIG. 5A, first optical sensor 508a is employed to image third visual indicator 512c, second optical sensor 508b is employed to image fourth visual indicator 512d, and each of third optical sensor 508c and fourth optical sensor 508d are employed to image visual indicators 512a and 512b. As detailed above, the employment of the specific optical sensor for imaging may be determined based on the availability in the field of view, based on history (i.e., which sensor was used in previous cycles), or based on optimizing accuracy and occlusion.

Referring to FIG. 5B, coordinate systems defined by each of the objects are shown: A first coordinate system (xₐ, yₐ, zₐ) is defined by (i.e. attached to, or associated with) first object 50a, a second coordinate system (x_{b}, y_{b}, z_{b}) is defined by (i.e. attached to, or associated with) second object 50b, and a third coordinate system (x_{c}, y_{c}, z_{c}) is defined by (i.e. attached to, or associated with) third object 50c.

It is to be understood that the objects described in FIGS. 4 and 5A-B (e.g., first object, second object and third object), are not necessarily included or are part of the described systems.

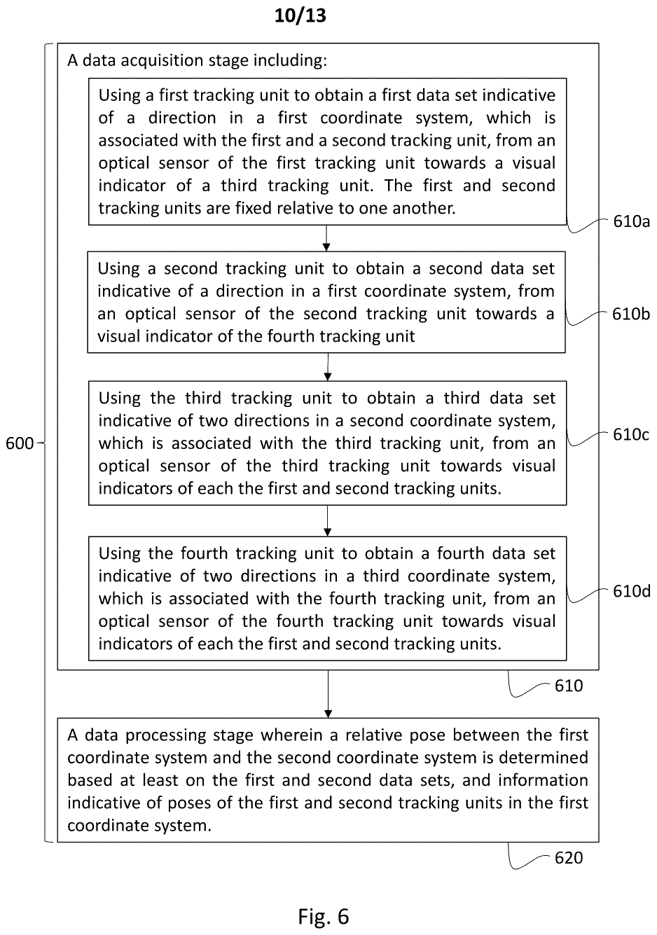

According to an aspect of some embodiments, there is provided an optical tracking-based method for determining a relative pose between a first coordinate system and each of a second coordinate system and a third coordinate system. FIG. 6 presents a flowchart of such a method 600, according to some embodiments. Method 600 may be implemented using an optical tracking system, such as system 400. According to some embodiments, method 600 includes:
A data acquisition stage 610 including:
   A substage 610a, wherein a first tracking unit (e.g. first tracking unit 502a) is used to obtain a first data set indicative of a direction in a first coordinate system, from an optical sensor of the first tracking unit towards a visual indicator of a third tracking unit (e.g. third tracking unit 502c), wherein the first coordinate system is associated with the first tracking unit and with a second tracking unit that is fixed relative to the first tracking unit.
   A substage 610b, wherein the second tracking unit (e.g. second tracking unit 502b) is used to obtain a second data set indicative of a direction in a first coordinate system, from an optical sensor of the second tracking unit towards a visual indicator of a fourth tracking unit (e.g. fourth tracking unit 502d).
   A substage 610c, wherein the third tracking unit is used to obtain a third data set indicative of two directions in a second coordinate system, which is associated with the third tracking unit, from an optical sensor of the third tracking unit towards visual indicators of each of the first and second tracking units.
   A substage 610d, wherein the fourth tracking unit is used to obtain a fourth data set indicative of two directions in a third coordinate system, which is associated with the fourth tracking unit, from an optical sensor of the fourth tracking unit towards visual indicators of each of the first and second tracking units.
A data processing stage 620, wherein relative poses between the first coordinate system and each of the second and third coordinate systems are determined based at least on the first, second, third, and fourth data sets, and information indicative of poses of the first and second tracking units in the first coordinate system.

In some embodiments wherein method 600 is used to provide real-time tracking, substages 610a, 610b, 610c, and 610d are performed substantially simultaneously (e.g., some or all of the substages may be performed simultaneously, and some or all of the substages may be performed in sequence).

According to some embodiments, method 600 may further include an initial stage wherein the first and second tracking units are fixed relative to one another. According to some embodiments, the first and second tracking units may be fixed relative to one another by fixing each to a first object (e.g. first object 50a). According to some embodiments, the third tracking unit may be fixed relative to a second object (e.g. second object 50b) and/or the fourth tracking unit may be fixed relative to a third object (e.g. second object 50c).

Data processing stage 620 may be implemented as explained above with respect to the operation of computer hardware 404. In particular, data processing stage 620 may be implemented using computer hardware, such as computer hardware 404.

According to some embodiments, data processing stage 620 may further include determining a relative pose between the second and third coordinate systems at least based on the relative pose between the first and second coordinate systems and the relative pose between the first and third coordinate systems.

According to some embodiments, data processing stage 620 may additionally include initial processing substages, implemented prior to the determination of the relative pose between the first coordinate system and each of the second and third coordinate systems. The initial processing substages may include:
A first initial processing substage, wherein the tracking data, obtained by the first tracking unit, is used to determine a direction (e.g. the direction indicated by arrow U₁ in FIG. 5A) from a first optical sensor thereof (e.g. first optical sensor 508a) to a visual indicator (e.g. third visual indicator 512c) of the third tracking unit.
A second initial processing substage, wherein the tracking data, obtained by the second tracking unit, is used to determine a direction (e.g. the direction indicated by arrow U₂ in FIG. 5A) from a second optical sensor thereof (e.g. second optical sensor 508b) to a visual indicator (e.g. fourth visual indicator 512d) of the fourth tracking unit.
A third initial processing substage, wherein the tracking data obtained by the third tracking unit is used to determine two directions (e.g. the directions indicated by arrows U₁′ and U₂′ in FIG. 5A) from an optical sensor thereof (e.g. third optical sensor 508c) to a visual indicator (e.g. first visual indicator 512a) of the first tracking unit and a visual indicator (e.g. second visual indicator 512b) of the second tracking unit, respectively.
A fourth initial processing substage, wherein the tracking data obtained by the fourth tracking unit is used to determine two directions (e.g. the directions indicated by arrows U₁″ and U₂″ in FIG. 5A) from an optical sensor thereof (e.g. fourth optical sensor 508d) to a visual indicator (e.g. first visual indicator 512a) of the first tracking unit and a visual indicator (e.g. second visual indicator 512b) of the second tracking unit, respectively.

According to some such embodiments, wherein the first and second tracking units are fixed relative to a first object and the third and fourth tracking units are fixed relative to a second and third objects, respectively, and the relative pose between the first object and each of the second and third objects is to be determined, the relative pose between the first object and the second object is computed at least based on (a) the direction determined in the first initial processing substage and the two directions determined in the third initial processing substage, and (b) the information indicative of the relative poses between the first object and each of the first and second tracking units, and of the relative pose between the second object and the third tracking unit, as explained above with respect to system 400. Similarly, the relative pose between the first object and the third object is computed based on (a′) the direction determined in the second initial processing substage and the two directions determined in the fourth initial processing substage, and (b′) the information indicative of the relative poses between the first object and each of the first and second tracking units, and of the relative pose between the third object and the fourth tracking unit, as explained above with respect to system 400. Here it is implicit that the internal structure of each of the tracking units is known.

Generally, the skilled person will readily perceive that system 400 may be used to determine the relative poses between a first coordinate system—defined by first tracking unit 402a and second tracking unit 402b and in which both are stationary (i.e. a coordinate system attached thereto, or associated therewith)—and each of a second coordinate system and a third coordinate system defined by (i.e. attached to, or associated with) third tracking unit 402c and fourth tracking unit 402d, respectively. In particular, when each of first tracking unit 402a and second tracking unit 402b is fixed relative to an object (e.g., first object 40a), then the first coordinate system is not only attached to tracking units 402a and 402b but also to the first object. Similarly, when third tracking unit 402c is attached to a second object (e.g., second object 40b), then the second coordinate system is not only attached to third tracking unit 402c but also to the second object. When fourth tracking unit 402d is attached to a third object (e.g. third object 40c), then the third coordinate system is not only attached to fourth tracking unit 402d but also to the third object. Thus, when first and second tracking units 402a and 402b are fixed relative to a first object (e.g. first object 10a), third tracking unit 402c is fixed relative to a second object (e.g. second object 40b), and fourth tracking unit 402d is fixed relative to a third object (e.g. third object 40c), then specification of the relative poses between the first coordinate system and each of the second and third coordinate systems (together with specification of the poses of the first object in the first coordinate system, the second object in the second coordinate system, and the third object in the third coordinate system) allows determining the relative poses between the first object and each of the second and third objects (and therefore also between the second object and the third object). Similarly, the skilled person will readily perceive that method 600 may be used to the same end.

According to some embodiments, the employment of the optical sensors in first tracking unit 402a, second tracking unit 402b, third tracking unit 402c and fourth tracking unit 402d is synchronized in time such that all optical sensors are employed simultaneously (e.g., the integration times of the optical sensors are synchronized). According to some embodiments, the optical sensors are not employed simultaneously. In both embodiments, if the light emitters that are generating the light emitted or reflected by the visual indicators are employed to emit light in pulses (e.g., when the light emitters are LEDs, they can be employed to emit pulses of light), the light pulses are employed simultaneously with the optical sensors that are opposite to the emitting or reflecting visual indicators and are configured to detect the emitted or reflected light. According to some embodiments, the visual indicator of tracking unit 402c is employed at a first time period, and the visual indicator of tracking unit 402d is employed at a second time period (e.g., when the light emitters generating the light emitted or reflected by the visual indicators are employed to emit light in pulses). In some of these embodiments, the first and second periods of time can be separated such that there is no overlap in time between the two periods. As a non-limiting example, the first period of time can be between time 0 and time 0.1 milliseconds (e.g., time within a tracking cycle), and the second period can be between time 0.1 and time 0.2 milliseconds. In these embodiments, the time separation can facilitate the identification of the source of the light detected in the optical sensors of first tracking unit 402a and second tracking unit 402b, for instance when both third tracking unit 402c and fourth tracking unit 402d are within the FOV of one or both optical sensors. In either of the above embodiments, each of the optical sensors of third tracking unit 402c and fourth tracking unit 402d (and both visual indicators of first tracking unit 402a and second tracking unit 402b) can be employed either simultaneously or not simultaneously with the optical sensors of first tracking unit 402a and second tracking unit 402b.

According to some embodiments, system 400 allows for periodic verification of the validity of the determined relative poses. The validity of the relative pose between the first coordinate system and the second coordinate system can be verified by simultaneously employing both the sensor of the first tracking unit and the sensor of the second tracking unit to image the visual indicator of the third tracking unit (as illustrated in FIG. 2B). In other words, once in every predetermined period (such as, for example, once in every second or once in every 10 seconds), the relative pose between the first and second objects can be determined twice. The first relative pose can be determined based on the data sets obtained by the first and third optical sensors, and the second relative pose can be determined based on the data sets obtained by the second and third optical sensors (e.g., this is possible only when the third tracking unit is within the FOV of both the first and the second optical sensors). In an event where both relative poses are not identical (e.g., up to an allowed tolerance), the system can alert the user (such as, for example, a surgeon using the VGS system) that something went wrong, and the user should take corrective measures. Such a scenario can happen, for example, when small blood droplets partially cover one of the optical sensors and/or one of the visual indicators, causing an optical distortion of the image of the visual indicator obtained by the optical sensor. Similarly, the validity of the relative pose between the first coordinate system and the third coordinate system can be verified by simultaneously employing both the sensor of the first tracking unit and the sensor of the second tracking unit to image the visual indicator of the fourth tracking unit. This is another advantage of the inherent redundancy of the modular optical tracker.

According to some embodiments, first tracking unit 402a and second tracking unit 402b can be employed for tracking the relative poses between first object 40a and more than two other objects, by reducing the effective rate of tracking. For instance, tracking units 402a and 402b can be employed for tracking second, third, fourth and fifth objects. As a non-limiting example, the tracking system described herein can have a cycle of 8.3 milliseconds, allowing tracking second and third objects relative to first object 40a at a rate of 120 cycles per second (e.g., tracking according to the method described above with respect to FIGS. 4-6). Tracking units 402a and 402b can be employed for tracking the relative pose between the first object and the second and third objects every other cycle (e.g., every 16.6 milliseconds instead of every 8.3 milliseconds), and tracking the relative pose between the first object and the fourth and fifth objects at the remaining cycles. In this example the tracking system is still employed at a rate of 120 cycles per second while effectively tracking each pair of objects at a rate of 60 cycles per second. Similarly, six objects can be tracked (e.g., relative to the first object 40a) at 40 cycles per second, and so on, by alternating between tracking couples/pairs of objects. It is noted that these embodiments are implemented with light emitting visual indicators (e.g., LEDs, not reflectors) which are employed in pulses that are synchronized accordingly (e.g., LEDs in the various tracking units are employed only in appropriate cycles).

According to some embodiments, the modular optical tracking system disclosed herein may be used for visor guided surgery (VGS) procedures. In VGS procedures, a head mounted display (HMD, for example, a see-through display) can allow augmenting the user's view of the subject (e.g., the patient), with various layers of data, such as, anatomical features and other virtual objects, to be presented to the user, as if the patient's body were partially transparent.

In some embodiments, the tracking system disclosed herein is combined with a head-mounted display (e.g., HMD) system, for displaying various layers of data/views, including, for example, augmented reality (AR) views, to provide the user with enhanced views, navigation and operational tools throughout the surgical procedure. The term AR as used herein is directed to include any information/data that can be overlaid on the “real world” view. In some embodiments, the AR views displayed by the combined display system can include (i) patient-stabilized overlays (e.g., overlays that are displayed in a fixed location and/or orientation relative to the patient's body, and are continuously updated to compensate for the user's head movement) such as, for example, but not limited to: a) registered in-situ augmentations of patient anatomy (for example, 2D and/or 3D virtual representations of patient anatomy), b) patient-stabilized preplanning information (for example, virtual representations of preplanned tool trajectories, ablation volumes, biopsy target points, bone cutting surfaces, etc.), c) patient-stabilized virtual screens (for example, displaying navigation data, and/or picture information such as video from a camera, in a fixed location relative to the patient's body), d) patient-stabilized tissue enhancements, for example, enhancements determined based on cameras and/or other imaging modalities imaging the tissue (e.g., the cameras and imaging modalities can be tracked relative to the patient), such as, for example, a camera operating in the infrared range and imaging indocyanine green (ICG) that was injected to the patient, that can allow blood vessel enhancement, or an intraoperative optical coherence tomography (OCT) device that can allow enhancing residual tumor tissue, e) patient-stabilized virtual tags and/or notes, such as, for example, virtual post-it notes to remind the surgeon of planned procedural steps, and f) patient-stabilized virtual menus and/or buttons. A virtual button can be activated, for example, by directing the center of the HMD toward the button (gazing), for instance for a duration of more than 0.5 seconds. Activating a virtual button can, for example, evoke an HMD-stabilized virtual menu that can be operated, for example, by head gestures. Activating a virtual button and operating a virtual menu (navigating within the menu and activating menu items) can both be facilitated by tracking the HMD. (ii) HMD-stabilized information (e.g., information that is displayed in a fixed location in the display coordinates, and does not change as a function of head movement) such as, for example, but not limited to: a) textual information (such as patient name and date), b) picture information (such as video from a camera, preoperative imaging, intraoperative imaging), c) guidance information (for example, displaying navigation screens in a fixed location in the HMD display, i.e., not fixed relative to the patient), and d) HMD-stabilized menus, (iii) tool-stabilized overlays (e.g., overlays that are displayed in a fixed location and/or orientation relative to a tool, and are continuously updated to compensate for the tool's movement and for the user's head movement) such as, for example, but not limited to: a) a virtual model of the tool, for example for viewing the virtual tool when parts of the actual tool are occluded, and/or for verifying that the tool is accurately tracked and for assessing the AR accuracy by checking the alignment of the virtual tool with the actual tool, b) planned clinical information, for example a spherical volume, representing a planned ablation volume, centered around a tooltip, c) picture information locked to a tool, for example for viewing video generated by an ultrasound transducer, when the tool is an ultrasound transducer, such that the ultrasound image is fixed to the edge of the ultrasound transducer and accurately located relative to the imaged anatomy, (iv) tracker-stabilized overlays (e.g., overlays that are displayed in a fixed location and/or orientation relative to a tracking unit, and are continuously updated to compensate for the tracking unit's movement and for the user's head movement) such as, for example, but not limited to: overlays for facilitating optimal tracker placement (see further below) and for AR accuracy verification, and (v) world-stabilized information, such as, for example, but not limited to, world-stabilized virtual screens (for example, displaying navigation data, and/or picture information such as video from a camera, in a fixed location relative to the world), and world-stabilized virtual menus and/or buttons.

According to some embodiments, the tracking system can thus allow a user to view various types of AR data, such as surgical navigation data, via a see-through head mounted display (HMD), while maintaining the surgical field in view. In some embodiments, the HMD may include any optical see-through display design, including, but not limited to, combiner optics, waveguide optics, visor-projection optics, holographic optics, retinal projection optics, and light-field optics. In some embodiments, the HMD may include any video see-through display design. When the HMD is an optical see-through HMD, the real world can be directly viewed. For example, when the HMD comprises visor-projection optics, the real world can be directly viewed through the semi-transparent visor, and when the HMD comprises retinal projection optics, the real world can be directly viewed through the transparent or semi-transparent optics comprising the display, and/or around the display optics. Direct view through the transparent or semi-transparent optics is meant to encompass a direct view in any optical see-through display design. When the HMD user can directly see the surgical field within the boundaries of the display FOV, direct view (of the surgical field) is said to be allowed (e.g., either in an optical see-through HMD or in a video see-through HMD). For example, direct view is allowed in an optical see-through HMD that has a shutter when the shutter is at least partially open and at least part of the displayed image is black (e.g., black areas in the image appear as transparent to the HMD user), or alternatively when the brightness of the image displayed via the HMD is low enough to allow a direct view. As another example, direct view is allowed in a video see-through HMD when the image/s from the forward-looking HMD camera/s are displayed via the HMD.

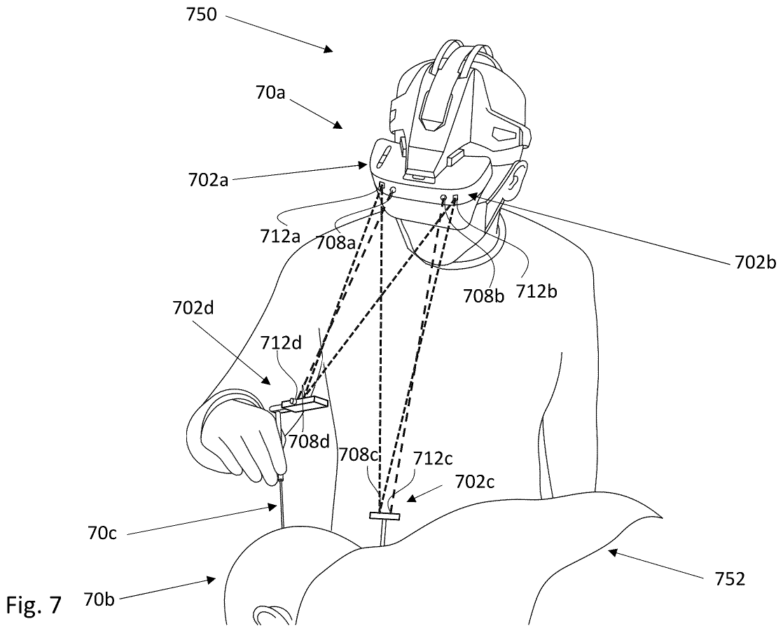

Reference is now made to FIG. 7, which presents a schematic illustration of a VGS system utilizing an optical tracking system for determining a relative pose between several objects, according to some embodiments. As shown in FIG. 7, a user (e.g., surgeon) 750, is wearing a head mounted display (HMD) 70a. HMD 70a (a first object) is associated with a first optical tracking unit 702a and a second optical tracking unit 702b, both embedded within HMD 70a. Patient 752 is shown with third optical tracking unit 702c associated with a body portion thereof. Tracking unit 702c is attached to a Mayfield clamp (not shown) to which the patient's head is fixed, such that tracking unit 702c is fixed with respect to the patient's skull (second object 70b). Further shown is a handheld surgical tool 70c (third object), which is associated with a fourth optical tracking unit 702d. First tracking unit 702a includes a first optical sensor 708a and a first visual indicator 712a. Second tracking unit 702b includes a second optical sensor 708b and a second visual indicator 712b. Third tracking unit 702c includes a third optical sensor 708c and a third visual indicator 712c. Fourth tracking unit 702d includes a fourth optical sensor 708d and a fourth visual indicator 712d. In the exemplary configuration illustrated in FIG. 7, the tracking system facilitates the determination of the relative poses between the first, second and third objects. As shown in FIG. 7, optical sensor 708b is employed to image visual indicator 712c, and third optical sensor 708c is employed to image visual indicators 712a and 712b. Optical sensor 708a is employed to image visual indicator 712d and fourth optical sensor 708d is employed to image visual indicators 712a and 712b. As detailed above, the employment of the HMD optical sensors for imaging may be determined based on the availability in the field of view, based on history (i.e., which one was used in previous cycles), or based on optimizing accuracy and occlusion. Thus, in such a setting, the sensor of one of the tracker units of the HMD can be utilized for tracking the patient tracker, and the sensor of the other HMD tracker unit can be utilized for tracking the tool tracker, while optionally, dynamically switch roles therebetween.

It is noted that in this example the surgical tool exhibits rotational symmetry around its long axis, and tracking unit 702d may be fixed to the tool with an adaptor that allows tracking unit 702d to swivel around the tool. If this is the case, only a partial 5-DOF pose is determined between the HMD (object 70a) and the tool (object 70c), although a full 6-DOF pose is determined between a joint coordinate system of tracking units 702a and 702b (such as the coordinate system of the HMD) and a coordinate system of tracking unit 702d.

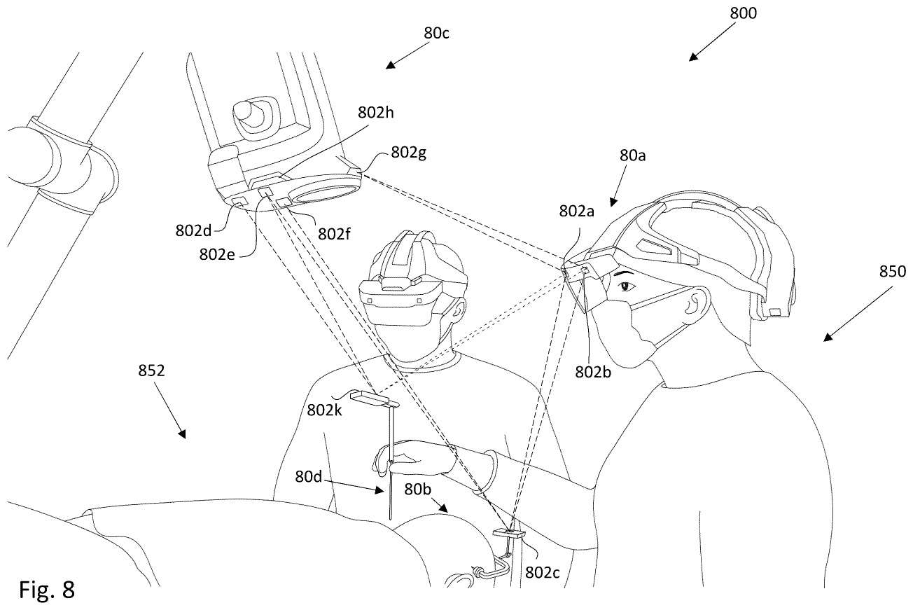

According to some embodiments, the VGS system can display, via the HMD, a tracking unit's FOV as a tracker-stabilized overlay (e.g., overlay conformal to the tracker). The FOV can be visually represented, for example, by a cone or a pyramid originating from the tracking unit's sensor and fixed to the tracking unit's coordinate system. In order to display the virtual FOV overlay, the relative pose between the tracking unit and the HMD needs to be tracked, either directly or indirectly (indirect tracking is described further below, with respect to FIGS. 8 and 9). For example, during the stage in which the surgeon fixes a tracking unit with respect to a patient's body part (such as the patient's skull in FIG. 7), when the surgeon moves and/or rotates the tracking unit, the virtual FOV overlay moves and/or rotates with it, assisting the surgeon to optimize the tracking unit's pose relative to the patient, a pose that will enable the tracking unit to track the surgeon's HMD throughout the procedure (e.g., the surgeon may change locations during the procedure, for instance the surgeon may move to the other side of the operating table). Once the tracking unit is fixed, the FOV overlay can be turned off. Similarly, when there are two or more discrete options for attaching a tracking unit to an instrument, or when a tracking unit can be attached to an instrument with an adjustable angle, the VGS system can display the tracking unit's FOV via the HMD, assisting the surgeon to fix the tracking unit in the optimal pose relative to the instrument, a pose that will optimize tracking coverage during the various stages of the procedure in which this instrument is used. In these embodiments, when two surgeons (e.g., users) are donning an HMD, FOV overlays can be displayed to both surgeons, for example to allow a senior surgeon to supervise over a resident during the stage of fixing the tracking unit to a patient. FOV overlays can also assist a surgeon that is still learning to work with the VGS system during later stages of the procedure. For example, FOV overlays can assist the surgeon to better position his or her head relative to the patient and/or relative to the instrument, so as to not lose tracking between the HMD and the patient/instrument.

According to some embodiments, when two surgeons (e.g., users) are donning an HMD, the VGS system can display, via the HMD of a first user, a second's user viewing direction as an overlay that is locked to the second user's HMD and optionally appearing as radiating from the second user's HMD. This can allow, for example, one surgeon to see the direction towards which the second surgeon is looking (and specifically the point of intersection of this direction with the surgical field). Similarly, according to some embodiments, the VGS system can display, via the HMD of a first user, an overlay on the actual surgical field representing a point that a second HMD user is looking at (e.g., the point can represent, for instance, the intersection of a ray originating in the second user's eye, passing through the center of the second user's display, and the surgical field). According to some embodiments, when the HMD comprises eye tracking, the VGS system can indicate the point that the second user is looking at based on the eye tracker. According to some embodiments, the VGS system can display to both users the second user's gazing direction and/or intersection of the gazing direction with the surgical field, thus providing the two users with a hands-free way of pointing out locations in the surgical field.

It is noted that various embodiments above and below may also be implemented with any see-through HMD and any tracking system, not necessarily a modular optical tracker.

According to some embodiments, the VGS system described herein can allow the surgeon to view navigation information via the HMD while maintaining the surgical field in their view (e.g., as opposed to standard surgical navigation systems, in which the navigation information is displayed via a standard monitor that requires the surgeon to shift his or her attention away from the surgical field). Navigation information typically includes a virtual representation of a tool with respect to 2D and/or 3D images of the anatomy, generated from the patient's imaging data, such as, for example, preoperative and/or intraoperative scans acquired by CT and MRI imaging devices. The 2D and/or 3D images can include, for example, axial and sagittal planes that include the tool, and 3D segmented views. The imaging data is registered to the patient anatomy (e.g., to the patient tracking unit that is fixed relative to the anatomy) in a registration process performed after fixing the patient tracking unit to the respective body part, and before the part of the procedure that requires navigation guidance. After registration, the pose of the imaging data coordinate system relative to the coordinate system of the patient tracking unit is known. Thereafter, the navigation information can be generated based on the determined pose of the tool (object 70c) relative to the patient tracking unit (and hence relative to the body part, object 70b), that is determined based on tracking the relative poses between the HMD (object 70a) and the body part (object 70b), and between the HMD (object 70a) and the tool (object 70c).

The navigation information can be displayed, for example, as patient-stabilized virtual screens (e.g., screens that are fixed relative to the surgical field) or as world-stabilized screens (e.g., typically the patient is motionless during a surgical procedure, so world-stabilized screens and patient-stabilized screens can be functionally similar). As another example, the navigation information can be HMD-stabilized (e.g., displayed in a fixed position in the HMD display). In some embodiments, the FOV of the HMD tracking units is large enough (for example, 120 degrees vertical FOV) to allow for tracking between the HMD and each of the patient and the tool even when the surgeon's head is not tilted downwards toward the patient. This can be advantageous with respect to ergonomics. In such cases, the surgeon can directly view the surgical field without tilting the head downwards by gazing below the display (e.g., by gazing downwards only with the eyes, for example by gazing below the visor, when the HMD comprises visor-projection optics), and can view the navigation information by comfortably gazing above the patient at the display (e.g., towards the image reflected from the visor, when the HMD comprises visor-projection optics). The latter mode of work can be referred to as head-up mode, as opposed to the former mode in which the surgeon gazes directly towards the surgical field, that can be referred to as head-down mode. In head-down mode the surgeon can optionally see at least part of the navigation information accurately overlaid on the patient's anatomy (e.g., in-situ AR, overlay that is conformal to the anatomy), whereas in head-up mode the navigation information is not superimposed with the patient and can appear as floating above the patient (e.g., non-conformal overlay).

According to some embodiments, the VGS system described herein can allow the surgeon to view, via the HMD, 2D and/or 3D imaging data and/or other guidance information registered to the actual patient anatomy (e.g., patient-stabilized in situ AR, guidance information conformal to the patient anatomy). In these embodiments, the guidance information is accurately superimposed with respect to the anatomy on the surgeon's view of the surgical field (e.g., on the direct view of the surgical field when the HMD is an optical see-through HMD, or on a live video of the surgical field when the HMD is a video see-through HMD). Such guidance information can include, for example, 2D and/or 3D virtual representations of patient anatomy (such as, for example, 3D segmentations of bones, vessels, and tumors, generated from imaging data, 2D planes generated from imaging data, etc.), and various preplanned guidance information (such as, for example, virtual representations of preplanned tool trajectories, ablation volumes, biopsy target points, bone cutting surfaces, etc.). In-situ AR overlays representing anatomical elements can appear as having the same scale (e.g., size), location and rotation as the corresponding anatomy they are superimposed on (e.g., conformal to the anatomy, as they appear via the HMD). In some embodiments, the in-situ AR guidance information can be generated based on the relative pose between the HMD (object 70a) and the body part (object 70b). In some embodiments, the in-situ AR guidance information is generated based on the relative pose between the HMD (object 70a) and the tool (object 70c). In some embodiments, the in-situ AR guidance information is generated based on the relative pose between the tool and the body part.

According to some embodiments, in-situ AR overlays are used to facilitate spine procedures. For example, during pedicle screw placement in open procedures, preplanned trajectories can be displayed as an overlay on the direct view of the vertebra. The surgeon can then align the tool (e.g., the actual tool as seen through transparent or the semi-transparent display, when the HMD is an optical see-through HMD) with the (virtual) trajectory. It is noted that this type of guidance requires tracking the HMD relative to the anatomy and does not require tracking the tool relative to the anatomy (or the HMD). During pedicle screw placement in percutaneous procedures, the guidance overlay can also include a 3D model of the vertebra (with or without neighboring vertebrae). As another example, during laminectomy procedures the in-situ AR guidance can include, for instance, a virtual representation of the ligamentum flavum and virtual representations of nerve roots (e.g., these overlays can increase the surgeon's confidence and might allow the surgeon to be more aggressive with using various tools), a virtual representation of the facet joint or the orientation of the facet joint (e.g., this overlay can allow the surgeon to make more efficient cuts), and facet cysts (if they exist). According to some embodiments, in-situ AR overlays are used to facilitate brain procedures. For example, during brain procedures the in-situ AR guidance can include, for instance, virtual representations of anatomical tissue such as tumors, vessels, nerve tracts and ventricles, and virtual planning information such as trajectories for keyhole procedures, craniotomy outlines, and so on. According to some embodiments, in-situ AR overlays are used to facilitate arthroplasty procedures. For example, during arthroplasty procedures the in-situ AR guidance can include, for instance, virtual representations of anatomical tissue such as bones and blood vessels, and virtual planning information such as cutting planes, trajectories for drilling, guidance for facilitating alignment, and so on.

In some embodiments, the location of guidance (e.g., navigation) information that is not overlaid on the patient's anatomy (e.g., information that is not in-situ AR, or non-conformal to the anatomy), either in head-up or in head-down modes, is user configurable. For example, the user can adjust the location where patient-stabilized information is displayed above the patient for comfortable head tilt. In some embodiments, the size of navigation information is user configurable. In some embodiments, the angle of navigation information is user configurable. For example, the user can tilt 2D virtual screens that include 2D navigation planes and rotate a 3D view. In some embodiments, various navigation elements, both elements that are overlaid on the patient's anatomy (e.g., in-situ AR) and elements that are not (e.g., not in-situ AR), are user configurable. For example, elements can be turned on and off, element brightness can be changed (e.g., the brightness of different overlay elements can be separately controlled), and so on. For instance, in open spine surgery the surgeon can turn off a 3D model of the vertebrae in the head-down mode (e.g., the in-situ AR overlay of the 3D segmented model can be turned off since the surgeon can directly see the vertebrae in open surgery) and keep it only in the head-up mode. In some embodiments, the user can configure the VGS system to automatically toggle the display of some navigation elements between on and off states, or to continuously turn their brightness up and down, in order to allow him or her to verify the accuracy of the alignment between the overlay and the actual anatomy (e.g., when the navigation elements are in-situ AR overlays). In some embodiments, the various attributes described above can be automatically adjusted. For example, the location and/or orientation of navigation information can be automatically adjusted based on HMD location relative to the patient and overlay brightness can be automatically adjusted based on the level of ambient light (e.g., that can be determined, for example, based on various sensors in the HMD).

According to some embodiments, the VGS system described herein can allow the surgeon to view, via the HMD, tool-stabilized overlays (e.g., overlays conformal to the tool) such as, for example, but not limited to: a virtual model of the tool (e.g. for viewing the virtual tool when parts of the actual tool are occluded), planned clinical information (e.g., a spherical volume, representing a planned ablation volume, centered around a tooltip), picture information locked to a tool (e.g., for viewing video generated by a 2D ultrasound transducer, when the tool is an ultrasound transducer, such that the ultrasound image is fixed to the edge of the ultrasound transducer and is accurately located relative to the imaged anatomy), and 3D image information locked relative to a tool (e.g., generated based on a tracked 3D ultrasound transducer). Live image information (e.g., video generated by a tracked endoscope) can be projected on known models of anatomical structures (e.g., anatomical structures that are not directly visible, or not fully directly visible to the surgeon), and models of other tracked tools. As another example, multiple images generated by a tracked 2D ultrasound transducer can be displayed as “locked” to the anatomy while the transducer is dynamically moved, so as to generate a patient-registered 3D visualization of the ultrasound data (e.g., in some of these examples the patient is also required to be tracked relative to the HMD, and not only the tool). In some embodiments, various tool-stabilized overlays are user configurable. For example, elements can be turned on and off and elements' brightness can be changed (e.g., the brightness of different overlay elements can be separately controlled), and so on. For instance, a virtual model of the tool can be turned on and off or its brightness can be turned up and down, in order to allow the user to verify the accuracy of the alignment between the overlay and the tool. In some embodiments, the various attributes described above can be automatically adjusted and automatically toggled. In some embodiments, the tool-stabilized AR overlays can be generated based on the relative pose between the HMD (object 70a) and the tool (object 70c). In some embodiments, the tool-stabilized AR overlays can be generated based on the relative pose between the HMD (object 70a) and the body part (object 70b). In some embodiments, the tool-stabilized AR overlays can be generated based on the relative pose between the tool and the body part.

In further embodiments, such VGS system may be combined with one or more additional components, including, for example, a surgical microscope, a surgical exoscope, a robotic arm, an ultrasound transducer, an endoscope, a laparoscope, an intraoperative imaging device, a surgical light, an automated medical device (such as a medical surgical robot), each of which may be associated with one or more tracking units and accordingly the relative pose between the various objects can be tracked by the tracking system.

In some embodiments, two or more tracking units are embedded or attached to a surgical light that is suspended above the surgical field during a procedure. In these embodiments, for example, tracking the relative poses between the surgical light and the patient and between the surgical light and a tool can provide navigation information during parts of the procedure when the surgeon is not donning the HMD (and is viewing the navigation information via a standard monitor), or when the direct tracking between the HMD and the patient or tool is temporarily discontinued due to FOV occlusion or FOV coverage. In another example, tracking the relative poses between the surgical light and the patient and between the surgical light and an imaging device can facilitate registration.

According to some embodiments, two or more tracking units are embedded or attached to an intraoperative imaging device, such as, for example, a 2D fluoroscope, a 3D C-arm, or an intraoperative CT. In these embodiments, the relative pose between the imaging device and the patient tracking unit can be tracked concurrently with the imaging being performed (e.g., the relative pose can be tracked during the imaging, when the imaging device and the tracking system are synchronized, or can be sampled before and/or after the imaging, when the imaging device is at the initial and/or final imaging position). The tracking units in these embodiments can be fixed to the imaging device such that the patient tracking unit is in their FOV (and vice versa) during the imaging (or at least during part of the imaging). Tracking the relative pose between the imaging device and the patient tracking unit can allow the imaging data to be automatically registered with the patient tracking unit, assuming that the alignment between the imaging device and the tracking units that are fixed to it is pre-calibrated. According to some embodiments, tracking between the imaging device and the patient can be performed indirectly. For instance, by tracking the relative pose between the imaging device and an HMD donned by a user, and tracking the relative pose between the HMD and the patient. In these embodiments, one or more tracking units are embedded or attached to the intraoperative imaging device. In the example of indirect tracking with an HMD, the tracking unit/s can be fixed to the imaging device such that the HMD is in its FOV (and vice versa) when the surgeon is attending the surgical field and the imaging device is positioned for scanning the patient. According to some embodiments, tracking units are embedded or attached to an intraoperative imaging device such that both the patient and additional objects, such as the HMD and/or a surgical light, can be tracked. According to some embodiments, one or more tracking units are embedded or attached to the intraoperative imaging device facing up, allowing tracking between a surgical light and the imaging device. In these embodiments, the relative pose between the surgical light and the patient can also be tracked (e.g., when two or more tracking units are embedded or attached to the surgical light), allowing the imaging data to be automatically registered with the patient tracking unit (e.g., similarly to what has been described above).

According to some embodiments, ultrasound-based registration is performed using a tracked ultrasound transducer. For example, in spine surgery the relative pose between an ultrasound transducer and a patient tracking unit can be tracked while vertebrae are being scanned with the transducer (e.g., either by direct tracking between the transducer and the patient, or by tracking both the patient and the transducer, for instance via the HMD). In this example, registration of a vertebra to a preoperative imaging dataset (e.g., CT and/or MRI imaging) can be determined based on the scanned outer surface of the vertebra. In some embodiments, the HMD guides the surgeon through the registration by displaying the accumulated ultrasound data, indicating how to move the transducer in order to acquire missing data, and once registration is achieved for a vertebra, displaying a 3D model of the vertebra that is derived from the imaging dataset and guiding the surgeon to proceed to scan the adjacent vertebra (e.g., each vertebra is separately registered). The guidance can be displayed as an in-situ AR overlay on the direct view of the surgical field.

Reference is now made to FIG. 8, which presents a schematic illustration of an exemplary integrated visualization and guidance system 800 utilizing an inside-out/outside-in optical tracking system for determining a relative pose between several objects, according to some embodiments. As shown in FIG. 8, a user (e.g., surgeon) 850, is wearing a head mounted display (HMD) 80a. HMD 80a (a first object) is associated with a first optical tracking unit 802a and a second optical tracking unit 802b, both embedded within HMD 80a. Patient 852 is shown with third optical tracking unit 802c associated with a body portion thereof. Tracking unit 802c is attached to a Mayfield clamp to which the patient's head is fixed, such that tracking unit 802c is fixed with respect to the patient's skull (second object 80b). Further shown is an exoscope 80c (third object), which is associated with three downward-looking tracking units 802d, 802e, and 802f and four sideway-looking tracking units (of which 802g, and 802h are shown). All tracking units include an optical sensor and a visual indicator. In the exemplary configuration illustrated in FIG. 8, the tracking system facilitates the determination of the relative poses between the first, second and third objects.

As shown in FIG. 8, two tracking units selected from downward-looking tracking units 802d, 802e, and 802f are employed together with tracking unit 802c in tracking the relative pose between the exoscope and the patient (e.g., an optical sensor in one of the two exoscope tracking units is employed and both visual indicators). The employment of the specific pair of tracking units, and the specific optical sensor to be employed within this pair, may be determined based on the availability in the field of view, based on history (i.e., which one was used in previous cycles), or based on optimizing accuracy and occlusion. Thus, in such a setting, one pair of tracker units selected from tracking units 802d, 802e, and 802f can be utilized for tracking the patient tracker (e.g., for tracking the relative pose between the exoscope and the patient's skull) during one part of the procedure, and another pair selected from tracking units 802d, 802e, and 802f can be utilized for tracking the patient tracker during another part of the procedure. Similarly, the selection of the optical sensor within the selected pair of tracking units to be employed for tracking can also be dynamically switched.

Further in FIG. 8, HMD tracking units 802a and 802b are employed together with tracking unit 802g to track the relative pose between the HMD and the exoscope (e.g., an optical sensor in one of the HMD tracking units is employed and both visual indicators). The selection of which of the sideway-looking exoscope tracking units (for example, 802g and 802h or other, not shown sideway facing exoscope tracking units 802i and 802j) is employed to track the relative pose between the HMD and the exoscope can be dynamically switched, for instance based on the availability in the field of view (e.g. in FIG. 8, the HMD is the FOV of tracking unit 802g, hence it is selected). For example, to determine which tracking unit is employed, system 800 can sequentially employ each of the sideway-looking exoscope tracking units concurrently with the HMD tracking units, until successful tracking is achieved by one of the sideway-looking exoscope tracking units. HMD tracking units 802a and 802b are also employed together with the patient tracking unit 802c to track the relative pose between the HMD and the patient (e.g., the optical sensor in the other HMD tracking unit, that is not employed for tracking the relative pose between the HMD and the exoscope, is employed and both visual indicators). Similar to as detailed above, the employment of the specific optical sensor may be determined based on the availability in the field of view, based on history (i.e., which one was used in previous cycles), or based on optimizing accuracy and occlusion. Thus, in such a setting, the sensor of one of the HMD tracker units can be utilized for tracking the patient tracker, and the sensor of the other HMD tracker unit can be utilized for tracking the exoscope, while optionally, dynamically switch roles therebetween. It is noted that the FOV of the HMD tracking units in the example depicted in FIG. 8 is large enough such that both the exoscope and the patient tracking units are in their FOV. As a non-limiting example, the FOV of each of HMD tracking units 802a and 802b can be about 120 degrees (vertical) by 80 degrees (horizontal).

FIG. 8 further shows a tool, fourth object 80d, and tracking unit 802k that is fixed with respect to the tool, also including an optical sensor and a visual indicator. As shown in FIG. 8, another pair of downward-looking tracking units selected from tracking units 802d, 802e, and 802f (e.g., other than the pair employed for tracking the relative pose between the exoscope and the patient) are employed together with tracking unit 802k for tracking the relative pose between the exoscope and the tool. Although in FIG. 8 different pairs of downward-looking exoscope tracking units are shown to be employed for tracking the relative pose between the exoscope and each of the patient and tool, the same pair can be utilized for tracking both poses. The selection of the specific pair of tracking units to be employed, and the specific optical sensor to be employed within this pair, can be dynamic as described above.

In some embodiments, integrated visualization and guidance system 800 utilizes in-out/out-in tracking for tracking a relative pose between the exoscope and a tool, such as tool 80d, as described above. In these embodiments, the tool can be fixed with a tracking unit comprising an optical sensor and a visual indicator. In some embodiments, integrated visualization and guidance system 800 utilizes out-in tracking for tracking a relative pose between the exoscope and a tool, such as tool 80d. In these embodiments, out-in tracking can be implemented using the same exoscope tracking units that are also employed for in-out/out-in tracking between the exoscope and the patient, and/or between the exoscope and the HMD. In some embodiments, integrated visualization and guidance system 800 utilizes out-in tracking for tracking a relative pose between the HMD and a tool, such as tool 80d.

In some embodiments, when integrated visualization and guidance system 800 utilizes out-in tracking for tracking a relative pose between the exoscope and a tool and/or between the HMD and a tool, the tool can be equipped with visual indicators (e.g., reflectors or light emitting, without an optical sensor), and at least one optical sensor in at least one of the exoscope tracking units or the HMD tracking units can be employed for detecting the visual indicators. Typically, in out-in tracking, a tracked tool is equipped with two or more visual indicators. Two visual indicators can be sufficient for determining a partial relative pose of a tool, for instance a 5-DOF pose of a tool exhibiting rotational symmetry around a long axis, and three visual indicators can be sufficient for determining a full 6-DOF relative pose of a tool. In some embodiments, when integrated visualization and guidance system 800 utilizes out-in tracking for tracking a relative pose of a tool, integrated visualization and guidance system 800 concurrently employs two or more tracking units selected from the exoscope tracking units 802d-802j, the HMD tracking units 802a and 802b, and the patient tracking unit 802c (e.g., the same tracking units that are also employed for in-out/out-in tracking between the exoscope, the HMD and the patient), for tracking one or more relative poses selected from the relative poses between the tool and each of the exoscope, the HMD and the patient. For example, an optical sensor in one of the exoscope tracking units and the optical sensor in the patient tracking unit can both be employed to image visual indicators of the tool. In this example, the visual indicators of the tool tracking unit can be LEDs arranged such that they are on a sphere (e.g., looking outward), such that different LEDs can be imaged by the different tracking units imaging the tool tracking unit from different directions. In this example, the relative pose between the patient and the exoscope is known (e.g. based on direct or indirect in-out/out-in tracking between the exoscope and the patient, as described herein below). Therefore the integrated visualization and guidance system 800 can combine the data set indicative of a direction or directions from the optical sensor in the exoscope tracking unit towards some of the tool visual indicators with the data set indicative of a direction or directions from the optical sensor in the patient tracking unit towards some of the tool visual indicators (e.g., not necessarily the same visual indicators), to derive the relative poses between the tool and each of the exoscope and the patient.

In some embodiments, relative tracking between any pair of objects selected from the HMD, exoscope (or generally any other type of suitable camera assembly), and patient can be implemented by directly tracking the relative poses for two pairs of objects, and deriving the relative pose between objects of the third pair from these two relative poses (e.g., indirect tracking). For example, when the relative pose between the exoscope and the patient cannot be tracked directly, this pose can be derived by directly tracking the relative pose between the HMD and the exoscope and the relative pose between the HMD and the patient. The relative pose between the exoscope and the patient cannot be tracked directly when, for instance, there is an obstruction of the line-of-sight between the exoscope tracking units and the patient tracking unit, or when the exoscope is moved to a position or angle in which the FOV of patient tracking unit 802c does not cover the exoscope.

In some embodiments, relative tracking between any pair selected from the HMD, exoscope, and tool can be implemented by directly tracking the relative poses between objects in two of the three pairs, and deriving the relative pose between objects in the third pair from these two relative poses (e.g., in these embodiments the tool tracking unit comprises a sensor). For example, when the relative pose between the HMD and the tool cannot be tracked directly, this pose can be derived by directly tracking the relative pose between the HMD and the exoscope, and between the exoscope and the tool. The relative pose between the HMD and the tool cannot be tracked directly when, for instance, there is an obstruction of the line-of-sight between the HMD tracking units and the tool tracking unit, or when the tool is held at an angle in which the FOV of tool tracking unit 802k does not cover the HMD.

As described herein above, the HMD tracking units in the example of FIG. 8 are employed for tracking the relative poses between the HMD and each of the exoscope and the patient. However, one of the HMD tracking units (e.g., the optical sensor in one of the HMD tracking units) can be employed for tracking the relative pose between the HMD and the tool, instead of the exoscope or the patient. Alternatively, as described herein above, HMD tracking units can be employed for tracking the relative pose between the HMD and the tool (and optionally, also additional objects) while also employed for tracking the relative pose between the HMD and each of the exoscope and the patient, by tracking one or more of the relative poses at a reduced rate (e.g., by alternating between tracking one object at one tracking cycle and tracking a second object at the next tracking cycle, and so on, as described above). The same employment logic can be implemented also for the exoscope tracking units and any other object that is fixed with one or more tracking units.

FIG. 8 depicts an exoscope suspended above the patient by a robotic arm. In some embodiments, the exoscope is suspended by a robotic arm. In some embodiments, the exoscope can be suspended by any other mechanism. In some embodiments, instead of an exoscope, the system can be implemented with a surgical microscope. In these embodiments, tracking units can be configured to be attached to an existing surgical microscope or alternatively, tracking units can be embedded within the surgical microscope. In some of these embodiments, one or both optical channels (e.g., the left and right optical channels) can be equipped with a camera/s for acquiring live magnified images of the surgical field through the microscope optics. In some of the embodiments, the camera or cameras are embedded within the microscope. In some of the embodiments, the camera or cameras are attached to the microscope (e.g., as an add-on using a beam splitter or beam splitters, or as a replacement to the microscope eyepiece or eyepieces). In some of the embodiments the video stream (or streams) from the camera (or cameras) is viewed via the HMD. In some of these embodiments, the video stream (or streams) can be augmented as described herein below.

It is to note that embodiments described herein with respect to an exoscope can be similarly implemented using any suitable camera assembly, such as, a surgical microscope equipped with a camera or a plurality of cameras.

In some embodiments, an “exoscope tracking unit” may also refer to a “microscope tracking unit” or “camera tracking unit”, as both the exoscope and the microscope as described herein are configured to generate a live image via a camera.

According to some embodiments, the integrated visualization and guidance system(s) disclosed herein utilizing an inside-out/outside-in optical tracking system, for determining a relative pose between several objects, do not essentially include an HMD, and corresponding images may be viewed by a user(s) on a screen (such as, a 2D or 3D screen/monitor/display). In such instances, other objects of the system associated with respective tracking units may be used for relative tracking between various objects (such as, user, patient, tool(s), camera(s), etc.).

FIG. 8 depicts two surgeons donning an HMD. Tracking between the second HMD and each of the exoscope, patient, tool and the first HMD can be implemented with any of the tracking units, based on which of the opposite tracking units are within the fields-of-view of the various other tracking units. As described herein above, when there is no direct line-of-sight between two objects (e.g., between tracking units fixed to two objects), a relative pose can be derived by directly tracking alternative pairs of objects. For example, when the line-of-sight between the second HMD and the patient is obstructed, the relative pose between them can be derived from the relative poses between the second HMD and the exoscope and between the exoscope and the patient, or from the relative poses between the second HMD and the first HMD and between the first HMD and the patient. It is noted that when tracking the relative pose between two HMD units, employing only three of the four tracking units (e.g., employing two opposite optical sensors and three visual indicators of the three tracking units) is sufficient for determining the relative pose. As another example, when the line-of-sight between the second HMD and the tool is obstructed, the relative pose between them can be derived from the relative poses between the second HMD and the first HMD and between the first HMD and the tool.

In some embodiments, when two or more surgeons are donning an HMD, and each is using a footswitch for facilitating the control over different system functions, the system can automatically and dynamically pair each footswitch with a corresponding surgeon based on the surgeons' locations. The surgeons' locations can be determined by the relative poses between the surgeons and the patient and/or the relative poses between the surgeons and the exoscope (e.g., when an exoscope is used). In these embodiments, the locations of the various footswitches (e.g., relative to the patient, or relative to the exoscope) can be indicated to the system, for instance at the beginning of a procedure. For example, in spine surgery, two surgeons are standing at opposite sides of the surgical table, each uses his or her own footswitch, for instance by pressing the footswitch to evoke a virtual menu. When the surgeons switch places, each footswitch can be automatically paired to the correct surgeon (e.g., the surgeon now standing next to the footswitch), for instance based on tracking the relative poses between the surgeons and the patient. Thanks to the automatic pairing between the surgeons and the footswitches, the menu is displayed only in the HMD donned by the surgeon that pressed the footswitch.

In some embodiments, any of the optical tracking systems described herein above and below utilize tracking redundancies to identify and alert in cases of tracking inaccuracies. A tracking inaccuracy can occur, for example, when a blood droplet partially obscures an optical sensor or a visual indicator in a tracking unit. In these embodiments, the tracking system can determine a relative pose between two coordinate systems (e.g., coordinate systems of tracking units and/or objects) using more than one set of tracking units and/or optical sensors, and compare the determined poses. For instance, the relative pose between the HMD and the patient can be determined by direct tracking using one of the optical sensors of the HMD tracking units (e.g., and the patient tracking unit), and concurrently deriving this pose by tracking the relative poses between the HMD and the exoscope (e.g., using the other optical sensor in the HMD tracking units), and between the exoscope and the patient. In this example, the system can periodically determine the relative pose between the HMD and the patient using both these alternatives, and alert when the two determined poses differ (e.g., by more than a predefined threshold). In some embodiments, by utilizing the tracking redundancies, the tracking system can identify the source of inaccuracy and guide the user to rectify the problem. For instance, in the above example, after identifying a tracking inaccuracy the tracking system can switch the roles of the HMD tracking units (e.g., use the second optical sensor for the direct tracking and the first optical sensor for tracking via the exoscope). In this case, if after the switch the two determined poses change (e.g., by more than a predefined threshold), but one of the determined poses before the switch equals to one of the determined poses after the switch, this can indicate that there is an issue with one of the HMD tracking units. If the two determined poses do not change, it can indicate that the problem is not in the HMD, and the tracking system can proceed to determine the relative pose between the HMD and the patient using alternative tracking units, such as other tracking units in the exoscope or other patient tracking units (e.g., if such units exist), or using alternative optical sensors and/or visual indicators (e.g., if they exist, such as when using tracking units with a plurality of optical sensors and/or visual indicators, as describe herein above, for instance in the patient tracking unit).

In some embodiments, some or all of the tracking units 802d-802j are embedded within the exoscope, as shown in FIG. 8. In some embodiments, some or all of the tracking units 802d-802j are detachable and can be fixed to the exoscope. In some embodiments, fewer or more tracking units are embedded and/or can be fixed to the exoscope. In some embodiments, wherein tracking units can be fixed to the exoscope, the fixing is to an accurate mounting mechanism. In some embodiments, the exoscope is utilized without a drape. In some embodiments, the exoscope is utilized in surgery with a drape (e.g., a sterile cover). In some of the embodiments where the exoscope is utilized with a drape, the drape can have optical windows for the exoscope cameras (or, in the case of a surgical microscope as described herein below, for the optical channels of the microscope) and for the tracking units that are embedded within or attach to the exoscope beneath the drape. In some of the embodiments where the exoscope is utilized with a drape, the exoscope can have accurate mounting mechanisms that allow attaching tracking units from outside the drape without breaking sterility (e.g., the mounting mechanisms on the exoscope side are under the drape, and sterile tracking units are fixed above the drape).

Similarly, in some embodiments, some or all of tracking units 802a and 802b are embedded within the HMD, as shown in FIG. 8. In some embodiments some or all of tracking units 802a and 802b are detachable, and can be fixed to the HMD. In some embodiments, fewer or more tracking units are embedded and/or can be fixed to the HMD. In some embodiments, wherein tracking units can be fixed to the HMD, the fixing is to accurate mounting mechanisms.

In the example depicted in FIG. 8, the downward-looking and sideway-looking exoscope tracking units can have a relatively small FOV. As a non-limiting example, the FOV can be 90 degrees (e.g., diagonal FOV) or less, which can be enough to cover the patient and the tool (downward-looking tracking units) and one or more HMD units (sideway-looking tracking units), in various surgical settings in which the exoscope is suspended above the patient at various angles and distances. In an alternative configuration, the tracking units can be embedded and/or attached to the exoscope such that their FOVs covers both the region beneath the exoscope (e.g., including the patient and possibly also a tool or tools equipped with a tracking unit), and the region to the side of the exoscope (e.g., including one or more headsets, donned by one or more surgeons, respectively). As a non-limiting example, the FOV of such tracking units can be 120 degrees or more.

It is noted that the downward-looking tracking units are embedded within (or fixed to) the exoscope such that the distances between pairs of downward-looking tracking units support accurate tracking between the exoscope and each of the patient and tool. For example, the tracking units may be fixed to the exoscope with distances of, for example, 10-14 cm therebetween, corresponding to the distance between the exoscope and the tracked objects, that are typically at a distance of, for example, 30-80 cm from the exoscope during surgery. Thanks to the small form factor of the modular tracking units, embedding (or fixing) the tracking units within the exoscope is easy and the increase in size and weight of the exoscope due to the tracking units is negligible. This is especially true when the tracking units are embedded within the exoscope and therefore do not require dedicated housing and power supply (e.g., a battery). In these cases, for example, a tracking unit can consist of only a small printed circuit board (PCB) comprising an optical sensor (e.g., including its optics) and an LED, and some other small components.

In some embodiments, the inside-out/outside-in optical tracking system of FIG. 8 can be implemented with tracking units comprising at least one optical sensor and at least two visual indicators, such that the distance between the visual indicators is sufficient for accurate tracking as described herein above (e.g., these tracking units are larger in at least one dimension compared to the modular tracking units). Such tracking units can replace pairs of tracking units comprising at least a single visual indicator (e.g., modular tracking units). Such tracking units can be embedded, for example, in HMD 80a and/or in exoscope 80c. In some of these embodiments, the tracking units comprising two or more visual indicators can comprise at least two optical sensors, thus functionally they can be identical to a pair of modular tracking units. A tracking unit comprising a single optical sensor and two visual indicators can be employed for tracking a single object in full rate, or for tracking more objects in reduced rate (e.g., as described herein above for the modular tracking units). Additional tracking units may be required for providing redundancy similar to the redundancy offered by the implementation with modular tracking units.

In some embodiments, during part of a surgical procedure images (e.g., images including live video streaming) from the exoscope (or microscope) camera (or cameras) are displayed to the surgeon via the HMD. In some embodiments, during part of a surgical procedure images from the camera/s are not displayed via the HMD. In some embodiments, images from the camera/s are displayed and/or not displayed based on the head direction of the surgeon donning the HMD. For example, when the surgeon's head is directed towards the surgical field, display of images can be discontinued to allow the surgeon to view the surgical field directly through the semi-transparent HMD optics. As another example, when the surgeon's head is directed above the surgical field, display of images can be resumed. In some embodiments, HMD directions (e.g., surgeon's head directions) wherein images are displayed or not displayed can be configured by a user. In some embodiments, overlays configured to be superimposed on the direct view of the surgical field are displayed when the HMD is directed towards the surgical field. In some embodiments, when the HMD is a video see-through HMD, overlays on the direct view of the surgical field are overlaid on a live image from a camera (or cameras) embedded in (or fixed to) the HMD (e.g., coupled to the HMD). In some embodiments, when the HMD is a video see-through HMD, the live video from the camera (or cameras) embedded in the HMD is displayed when the HMD is directed towards the surgical field. In some embodiments, when the HMD is an optical see-through HMD, the HMD includes a shutter that has variable transparency, for occluding ambient light so as to improve the contrast of images (e.g., images from the exoscope, when viewing the exoscope video via the HMD, or other images displayed via the HMD). In some of these embodiments, the shutter can be switched (e.g., by demand or automatically) between close and open states. In some of these embodiments, the shutter transparency can be adjusted to any of multiple levels between the close and open states. High HMD transparency allows the user to directly view the surgical field. In some embodiments, when the HMD is an optical see-through HMD, the HMD includes a mechanical shutter that can be moved (e.g., manually or by a motor) between close and open states. In some embodiments, when the HMD is an optical see-through HMD, the HMD includes two shutters, one for each optical channel (e.g., the left and right optical channels of the HMD). In some embodiments, when the HMD includes a shutter, the shutter is automatically controlled according to the whether or not the surgeon is viewing images (e.g., images from the exoscope or other images that may require high contrast, such as the patient's imaging data). In some embodiments, when the HMD is an optical see-through HMD, there is no shutter in the HMD. In these embodiments, the HMD brightness can be sufficiently high for viewing images with high contrast without occluding ambient light (e.g., without darkening the background seen through the HMD).

In some embodiments, images from the exoscope (or microscope) are displayed via the HMD while concurrently allowing the user to directly view the surgical field. For example, images from the exoscope can be displayed in a patient-stabilized virtual screen positioned near the attended area of the surgical field but without occluding it. In this example, when the HMD is an optical see-through HMD, areas in the display image that do not include the virtual screen are black, to allow the user to directly view the surgical field (e.g., when a black image is displayed in parts of an optical see-through display, these parts can appear to be transparent to the HMD user), and when the HMD is a video see-through HMD, the virtual screen can be displayed as a patient-stabilized overlay on the live video from the camera (or cameras) embedded in the HMD. In another example, when the HMD is an optical see-through HMD, images from the exoscope can be displayed only in part of the display (e.g., in a picture-in-picture format over a black background), such that the surgical field can be directly viewed through other parts of the display. Similarly, when the HMD is a video see-through HMD, images from the exoscope can be displayed as an HMD-stabilized picture-in-picture overlaid on the live video from the camera (or cameras) embedded in the HMD. In some embodiments, when the surgeon's head is directed above the surgical field, the image from the exoscope is displayed on the entire HMD display (e.g., such that it occludes the view through the display in an optical see-through HMD). In some embodiments, when the surgeon's head is directed towards the surgical field, the image from the exoscope is displayed such that it allows the user to directly view the surgical field (e.g., in a picture-in-picture or in a virtual screen, either in an optical see-through or a video see-through HMD).

The embodiments in which images from the exoscope are displayed via the HMD while concurrently allowing the user to directly view the surgical field can facilitate situation awareness. For instance, when tools are brought into the surgical field, the surgeon can glance downwards to see the entire surgical field and safely bring in the tool, while simultaneously being able to see when the tool enters the FOV of the magnified image and return to viewing the magnified image on the entire HMD display (and hence with better resolution), by rotating the head upwards again. In some embodiments, enhanced situation awareness can be provided to the surgeon while displaying the magnified image on the entire HMD display. In these embodiments, a low magnification image of the entire surgical field can be displayed as a picture-in-picture in the magnified image. For example, this can allow the surgeon to comfortably bring a tool into the surgical field by viewing the low magnification image until the tool is seen in the highly magnified image. In some embodiments, a low magnification image of the entire surgical field can be displayed instead of the magnified image, and switching between the magnified image and the low magnification image is done automatically, for instance based on detecting a tool or a hand entering the FOV of the low magnification image or exiting the FOV of the magnified image. These embodiments can be implemented in various ways. For example, the low magnification image (e.g., preferably, but not necessarily, a stereoscopic image) can be acquired by an additional camera (or cameras), other than the main exoscope cameras (e.g., additional camera/s that are embedded with, or fixed to the exoscope cameras). As another example, the main exoscope cameras can acquire ultra-high-resolution images of the entire surgical field (e.g., a low magnification image), and the magnified images can be generated by displaying only regions-of-interest taken from these ultra-high-resolution images (e.g., without compromising the resolution of the displayed image, as the acquired images have a resolution that is much larger than the resolution of the display). In this example, the low magnification image of the full field of view that is displayed as a picture-in-picture can be generated from the full ultra-high-resolution images, for instance by binning. In any of these embodiments, the low magnification image can be automatically displayed, for example when a motion of a tool or a hand entering the FOV of the low magnification image or exiting the FOV of the magnified image is detected. Similarly, in some embodiments the transition between viewing the magnified image on the entire HMD display and viewing the magnified image in a picture-in-picture or in a virtual screen, can also be based on detecting inward or outward motion (e.g., as opposed to transition based on the direction of the surgeon's head). In these embodiments, the surgeon can view the magnified image on the entire HMD display while gazing downwards towards the surgical field, and the system will automatically switch to displaying the magnified image in a picture-in-picture or in a virtual screen when inward or outward motion of a tool or a hand is detected.

The embodiments in which images from the exoscope are displayed via the HMD while concurrently allowing the user to directly view the surgical field can also allow the surgeon to use the exoscope as an alternative to loupes. In some embodiments, in addition to the range of magnification required for microsurgical procedures, the exoscope also covers the range of magnification provided by typical surgical loupes (for example, between 2.533 and 5.5×). In these embodiments, although the magnified image is not displayed over the entire display of the HMD (e.g., and therefore the image resolution is degraded with respect to using the entire display, for instance when the surgeon is looking away from the surgical field), this method of visualization can provide sufficient resolution for some steps of a procedure. In some embodiments that allow the surgeon to use the exoscope as an alternative to loupes, images from the exoscope are displayed in a virtual screen, and the location and/or orientation of the virtual screen can change according to the surgeon's viewing perspective. In some embodiments that allow the surgeon to use the exoscope as an alternative to loupes, images from the exoscope are displayed in a picture-in-picture format. In both these embodiments, the surgeon can comfortably view both the surgical field (e.g., directly), and the magnified image of the surgical field. The embodiments in which the user can directly view the surgical field can be advantageous with respect to surgical loupes since they allow the user to view the attended region in the surgical field either directly, without magnification, and indirectly, as a magnified view via the HMD (e.g., as opposed to loupes, in which the magnified view occludes the direct view). In some embodiments, the HMD is focused to a typical working distance, so there is no strain on the eyes when they switch between viewing the direct view and the magnified view. In some embodiments, the exoscope location and/or orientation changes (e.g., automatically, with or without an enablement input from the user) according to the surgeon's viewing perspective, thus allowing the surgeon to freely move and change his or her viewing perspective, similar to working with loupes (e.g., by tracking the surgeon's HMD, the system can determine the point the surgeon is looking at and move the exoscope so it images this point from the surgeon's perspective). It is noted that the embodiments in which images from the exoscope (or microscope) are displayed in a patient-stabilized virtual screen can be alternatively implemented with a world-stabilized virtual screen. The implementation can be based, for example, on an additional tracker unit that is fixed with respect to the world (such as a tracking unit or units embedded in the surgical light), or based on another tracking capability such as inertial tracking (e.g., using an IMU in the HMD). It is also noted that in the embodiments in which images from the exoscope (or microscope) are displayed in a patient-stabilized or world-stabilized virtual screen or in a PIP format, allowing the user to directly view the surgical field and concurrently view the images from the exoscope, either the direct view or the images from the exoscope, or both, can be augmented with guidance information as described below.

In some embodiments, images from the exoscope (or microscope) camera/s are augmented with guidance information that is generated based at least on one or more of the relative poses between pairs selected from the HMD, the exoscope (or microscope), and the patient. In some embodiments, the guidance information is generated based at least on one or more relative poses between a tracked tool (such as tool 80d in FIG. 8) and any of the HMD, the exoscope (or microscope), and the patient. In some embodiments, the guidance information is generated based at least on preoperative and/or intraoperative imaging data of the patient. In some embodiments, the guidance information is generated based at least on preoperative and/or intraoperative imaging data of the patient that is registered to the patient anatomy. In some embodiments, the guidance information is generated from raw imaging data of the patient. For example, planes generated from a 3D imaging dataset (e.g., axial, sagittal, coronal, and/or oblique planes generated from CT and/or MRI scans) can be overlaid on the exoscope image (e.g., overlaid on one of the two channels of the stereoscopic image or on both). In some embodiments, the guidance information is generated from processed imaging data of the patient. For example, segmentations generated from a 3D imaging dataset (e.g., segmentations of blood vessels, tumors, bones, and so on, generated from scans by a CT, MRI, and/or another imaging device) can be overlaid on the exoscope image. In this example, two rendered images of a segmented 3D model can be overlaid on the two channels of the stereoscopic image, each rendered from the perspective of the corresponding camera. For instance, in minimally invasive spine surgery using a cannula or retractors, a 3D model of the spine (e.g., with or without tags indicating the various vertebral levels) can be displayed outside of the cannula or the retractors (e.g., as they appear in the exoscope image) to enhance the surgeon's spatial orientation. In these embodiments, the guidance information can be generated such that it fits the points-of-view of the exoscope cameras and the scale of the magnified images. For example, images of a 3D model of the spine can be rendered from the same direction as the direction exoscope images are acquired from, and their scale can fit the magnification level of the image the user is viewing (e.g., when the exoscope utilizes digital magnification the acquired images and the viewed images do not necessarily have the same magnification, as the viewed images can be a region of interest within the acquired images). As such, the magnification level of the guidance information can be automatically adjusted whenever the exoscope magnification level is changed. It is noted that these embodiments, as well as other embodiments above and below, can be implemented using any type of tracking and not necessarily a modular optical tracker. It is also noted that these embodiments, as well as other embodiments above and below, can be implemented using any type of display, and not necessarily a head-mounted display. As further examples, the intraoperative imaging data can be generated by an infrared camera or by an optical coherence tomography (OCT) device that is fixed to, associated with (or integrated with) the exoscope cameras. In some embodiments, the guidance information comprises planning data that was generated (e.g. manually and/or automatically) based at least on imaging data of the patient. Planning data can include, for example, planned trajectories, planned volumes (e.g., volumes for ablation, volumes designating keep-out zones), planned incisions, planned locations for implants, and so on. In some embodiments, the guidance information is generated by a second surgeon donning a second HMD, a surgeon viewing the live image via a monitor in the operating room, or a surgeon that is not present in the operating room and is viewing the live image from a remote location. For example, the first surgeon can be a resident and the second surgeon can be a supervising surgeon that guides the resident by, for instance, controlling a pointer that is overlaid on the live image or, for instance, drawing a desired contour of an incision that is overlaid on the live image. According to some embodiments, when the HMD comprises eye tracking, the VGS system can indicate the point on the images from the exoscope that the second user is looking at based on the eye tracker. According to some embodiments, the VGS system can indicate the point to both users, thus providing the two users with a hands-free way of pointing out locations in the surgical field.

In some embodiments, the guidance information is augmented (e.g., overlaid) on the images from the exoscope (or microscope) in an accurate scale, location and orientation with respect to the anatomy (e.g., registered or conformal to the anatomy). In some embodiments, the scale of the guidance information is automatically adjusted according to the exoscope (or microscope) current magnification level. In some embodiments, the location and orientation of the guidance information is determined based on the relative pose between the exoscope (or microscope) and the patient. In some embodiments, the rendering of the guidance information is done from the perspective of the exoscope (or microscope) optical channel or channels, based on the relative pose between the exoscope (or microscope) and the patient. In some embodiments, the guidance information is augmented (e.g., overlaid) on the images from the exoscope (or microscope) as a picture-in-picture (e.g., not necessarily in an accurate scale, location and orientation with respect to the anatomy). For example, standard navigation planes, including a virtual representation of a tracked tool (such as tool 80d in FIG. 8) can be overlaid on the exoscope image at a fixed position relative to the image coordinate system (e.g., as a picture-in-picture). In these embodiments, the guidance information can be generated at least based on the relative pose between the tracked tool and the patient. In some embodiments, the guidance information is augmented (e.g., overlaid) on the images from the exoscope (or microscope) in a virtual screen at a fixed position relative to the patient coordinate system. For example, standard navigation screens can appear at a location that is fixed to the patient and unchanged when, for example, the image magnification is changed or when the exoscope perspective is changed (e.g., the orientation of the virtual screen can change when the exoscope perspective is changed, but the location can be fixed). In some embodiments, the guidance information is overlaid on the images from the exoscope (or microscope) in a virtual screen, and the position and/or orientation of the virtual screen is set based on the exoscope viewing perspective (e.g., the viewing angle relative to the surgical field). In some embodiments, the guidance information is overlaid on the images from the exoscope (or microscope) in a virtual screen, and the user can control the screen size, position and/or orientation relative to the surgical field as it appears in the exoscope image.

In some embodiments, the exoscope (or microscope) image can be augmented with one or more virtual windows into the patient's anatomy. For example, in open brain surgery, guidance information such as a virtual 3D segmented tumor located below the surface of the cortex can be displayed only within the borders of a virtual window located on the cortex (e.g., as it appears in the live image). Using a virtual window can improve the user's depth perception by providing occlusion and parallax cues. For instance, in the example of open brain surgery the surgeon can turn on the virtual window and the representation of the tumor (e.g., and other virtual elements such as vasculature) in order to see their location before proceeding with the procedure (e.g., after turning the overlays off). In some embodiments, the virtual window conforms to the shape of the anatomy. For example, in open brain surgery, the virtual window can conform to the surface of the cortex. In these embodiments, the shape of the anatomy can be derived, for instance, from the stereoscopic cameras of the exoscope, or from a 3D sensor or a depth sensor (e.g., a time-of-flight sensor or a structured light sensor) that is embedded in the exoscope. In some embodiments, the image within the borders of a virtual window can comprise only synthetic components, such as, for example, representation of tumors, vasculature, and so on, that are generated based on imaging data (e.g., preoperative and/or intraoperative imaging). In some embodiments, the image within the borders of a virtual window can comprise synthetic components along with the exoscope image (e.g., original or processed image). In these embodiments, the exoscope image within the borders of a virtual window can appear as being semi-transparent, causing the synthetic components to appear as being beneath the surface of the visible tissue seen in the exoscope image.

It is noted that in general, augmentations of the exoscope (or microscope) image can include augmenting only one of the stereoscopic channels (e.g., either the left or the right live image (exoscope) or optical channel (microscope)), and augmenting both stereoscopic channels (e.g., both the left and the right images/channels). Augmenting both stereoscopic images/channels can include augmenting the same overlay on both images/channels, and augmenting the different overlays on the left and right images/channels, such as, for example, when rendering a 3D model from the two points of view of the two exoscope cameras (in a stereoscopic exoscope). In a similar fashion, augmentations of an endoscope (or laparoscope) image (e.g., a tracked endoscope or laparoscope) can be implemented either with a typical 2D endoscope or with a stereoscopic endoscope. When augmenting an image of a stereoscopic endoscope, it can include augmenting either channel or both, and when both channels are augmented, the augmentation can be either identical or channel-specific.

In some embodiments, images from the exoscope (or microscope) camera/s are augmented with guidance information that is generated at least based on other images acquired by the exoscope (or microscope). For example, the exoscope can include a near infrared camera for enhancing the view of blood vessels based on ICG (indocyanine green) dye injected intravenously to the patient. Information generated from the live image of the near infrared camera can be superimposed on the visible image (e.g., the images from the exoscope) to enhance the blood vessels. In another example, similar enhancement is done to a tumor (e.g., using a fluorescent contrast agent and near infrared illumination).

In some embodiments, images from the exoscope (or microscope) camera/s are augmented with verification symbols as described in patent application, publication No. WO2022249190. Such verification symbols can indicate the validity of overlays (e.g., the accuracy of the location of the overlays relative to the anatomy) that were generated based on conversion of locations between coordinate systems.

In some embodiments, images from the exoscope (or microscope) camera/s are augmented while taking into consideration occlusion by elements in the image, as described in US20220354691. For example, the exoscope image can be segmented (e.g., in real-time), and the processor can superimpose (or not superimpose) an overlay only on selected segments. For instance, an overlay representing an anatomical element (e.g., a bone, a tumor, etc.) can appear as if a tool is occluding it by not superimposing the overlay on the tool segment in the image.

In some embodiments, overlays can be superimposed on the exoscope image such that they appear below the surface of the anatomy. For example, the overlay can be displayed as being semi-transparent, such that the surface of the anatomy that is above the element represented by the overlay can be viewed concurrently with the overlay. Combining the partial overlay transparency with a correct left-right disparity of the overlay (e.g., the difference between the overlay location in image displayed to the left eye of the HMD user and the overlay location in the image displayed to the right eye of the HMD user), which is different than the left-right disparity of the surface of the anatomy (e.g., since the overlay represents an element that is beneath the surface of the anatomy), can cause the overlay to appear as being at the correct depth below the surface of the anatomy. In some embodiments, when the overlay represents a 3D object (e.g., such as a tumor, a blood vessel, etc.), the overlay displayed to the left and right eyes of the HMD user can be each rendered from a different point of view (e.g., the two points of view corresponding to the points of view of the two cameras of the stereoscopic camera system), further enhancing the depth perception of the overlay relative to the surface of the anatomy. In some embodiments, overlays can be displayed within a virtual window. When using a virtual window (e.g., a virtual window which is also part of the overlay), the virtual window is displayed with a left-right disparity that matches that of the surface of the anatomy, and the surface of the anatomy within the borders of the virtual window can be completely occluded by the overlay (e.g., by displaying the overlay with zero transparency. Note that in these cases the augmented element is typically displayed within a substantially uniform background filling the area within the virtual window). In some embodiments, both the virtual window and the semi-transparent overlay can be used together, generating an effect of looking inside the anatomy through a “dirty” window. These embodiments can be preferred when it is desired to see both the surface of the anatomy and the augmented element (for instance, when the anatomy is the cortex, and the augmented element is a tumor underneath the surface of the cortex). The virtual window further enhances the depth perception, since the augmented element can be viewed only within the borders of the virtual window (e.g., a parallax effect is generated due to the different depths of the virtual window and the augmented element, for instance when the user moves the exoscope relative to the patient). The use of a virtual window without the semi-transparent overlay (e.g., displaying the overlay and the uniform background within the virtual window without transparency) can be preferred when there is no interesting information on the surface of the anatomy, for instance in a posterior minimally invasive spine surgery (e.g., when the surface of the anatomy is the patient's skin, and the augmented elements are vertebrae and other elements under the skin). It is noted that the augmented elements may represent not only anatomical tissues but also other physical elements such as, for example, surgical tools and implants, and virtual elements such as, for example, various preplanned guidance information.

In some embodiments, integrated visualization and guidance system 800 includes an endoscope and/or a laparoscope (or, in general, a tracked camera) instead of, or in addition to, an exoscope. In these embodiments a tracking unit, such as a modular tracking unit, can be fixed to the endoscope. In these embodiments, the relative pose between the endoscope and the patient can be tracked, for instance by tracking the relative pose between the HMD and the patient and the relative pose between the HMD and the endoscope. In general, all of the embodiments that are described herein with respect to an exoscope also apply (e.g., when applicable) to any tracked camera, including a tracked endoscope (or laparoscope). In some embodiments, an endoscope image can be augmented by displaying a 3D model of the anatomy around the endoscope image (e.g., around the boundaries of the circular video, when the endoscope camera has a round FOV), such that the 3D model fits the endoscope pose. For instance, in a spine procedure the system can display a 3D model of the spine around the endoscope video, and the 3D model is continuously rendered so its pose and scale fir the point of view of the endoscope and its distance from the anatomy. In some embodiments, the system can overlay any desired augmentation on the video itself, as described above with respect to overlays on the exoscope image, including displaying the augmentation in a virtual window, or such that it appears below the surface of the anatomy. For instance, in a spine procedure the overlay can include rendered models of vertebrae and, when available, various soft tissues such as vasculature and nerves segmented from MRI images. Typically, endoscopes provide 2D video but the overlay on the video can be either 2D or 3D (e.g., 3D overlay can be separately rendered for each channel). Similarly, when the endoscope provides stereoscopic video, the overlay can also be 2D or 3D. In some embodiments, 2D and 3D navigation information can be overlayed as pictures in picture over, or side-by-side with the endoscope image. For instance, 3D navigation information in a spine procedure can include a 3D model of the spine with a 3D representation of the endoscope (e.g., accurately located with respect to the spine), and optionally also a representation of the footprint of the endoscope image projected on a 3D model (e.g., the footprint represents the area on the anatomy that is displayed in the endoscope live image), or a representation of the FOV of the endoscope camera (for example, as a cone originating at the tip of the endoscope model). In some embodiments, images from the endoscope are displayed via the HMD while concurrently allowing the user to directly view the surgical field and/or the endoscope. For example, images from the endoscope can be displayed in a patient-stabilized virtual screen positioned near the surgical field or near the endoscope but without occluding it. As another example, when the HMD is an optical see-through HMD, images from the endoscope can be displayed only in part of the display, such that the surgical field and the endoscope can be directly viewed through other parts of the display. Switching between viewing the endoscope image on the entire HMD display and viewing the endoscope image while concurrently allowing the user to directly view the surgical field (and/or the endoscope) can be automatically controlled based on the pose of the endoscope relative to the patient, or based on the HMD pose relative to the endoscope or the patient. For example, the endoscope image can be switched to being displayed in a picture-in-picture format when the surgeon glances towards the endoscope, for instance while inserting a tool into the endoscope (e.g., which may require a direct view towards the endoscope). Switching between the two views can also be based on detecting that a tool is exiting the endoscope image (e.g., and hence, it can be assumed that the surgeon is replacing it with another tool).

It is noted that any of the above embodiments, such as, of augmenting exoscope and/or endoscope images with guidance information, can be implemented using any type of tracker/tracker system, that can be used to track the exoscope and/or the endoscope and/or a tool relative to the patient. Likewise, embodiments in which images from the exoscope and/or endoscope are displayed via the HMD, while concurrently allowing a user to directly view the surgical field, or embodiments in which the low magnification image is displayed instead of or concurrently with the magnified exoscope image, can be implemented with any type of suitable tracking systems.

It is further noted that embodiments in which images from an exoscope are augmented based on tracking the relative pose between the exoscope and other object(s) (such as, for example, patient, tool, HMD), can be implemented with any tracked camera (including an endoscope, for example). Likewise, embodiments where an HMD user can view images from the exoscope via an HMD while concurrently being able to directly view the surgical field (e.g., with or without augmentation of the direct view or the exoscope image), based on tracking the relative pose between the HMD and other object(s) (such as, for example, the exoscope, patient, tool), can be implemented with any camera (that may be utilized instead of the exoscope).

Referring again to FIG. 8, in some embodiments, the direct view of the surgical field (e.g., through the semi-transparent HMD optics, when the HMD is an optical see-through HMD, or via the image/s from the forward-looking HMD camera/s are displayed via the HMD, when the HMD is a video see-through HMD) is augmented with guidance information that is generated based at least on one or more of the relative poses between pairs selected from the HMD, the exoscope (or microscope), and the patient. In some of these embodiments, the guidance information is generated also based at least on one or more relative poses between a tracked tool (such as tool 80d in FIG. 8) and any of the HMD, the exoscope (or microscope), and the patient. In some embodiments, the guidance information is generated based at least on preoperative and/or intraoperative imaging data of the patient. In some embodiments, the guidance information is generated based at least on preoperative and/or intraoperative imaging data of the patient that is registered to the patient anatomy. In some embodiments, the guidance information is generated from raw imaging data of the patient. For example, planes generated from a 3D imaging dataset (e.g., axial, sagittal, coronal, and/or oblique planes generated from CT and/or MRI scans) can be overlaid on the direct view of the surgical field (e.g., displayed via the HMD to one of the user's eyes or both). In some embodiments, the guidance information is generated from processed imaging data of the patient. For example, 3D segmentations generated from a 3D imaging dataset (e.g., segmentations of blood vessels, tumors, bones, and so on, generated from scans by a CT, MRI, or any other imaging device) can be overlaid on the direct view of the surgical field. For example, two rendered images of a segmented 3D model are displayed via the HMD to both of the user's eyes, each rendered from the perspective of the corresponding eye. In some embodiments, the guidance information comprises planning data that was generated (e.g. manually and/or automatically) based at least on imaging data of the patient. Planning data can include, for example, planned trajectories, planned volumes (e.g., volumes for ablation, volumes designating keep-out zones), planned incisions, planned locations for implants, and so on.

In some embodiments, the guidance information overlaid on the direct view of the surgical field is generated by a second surgeon donning a second HMD, a surgeon viewing the live image from the exoscope via a monitor in the operating room (e.g., a 2D or a 3D monitor), or a surgeon that is not present in the operating room and is viewing the live image from the exoscope from a remote location. For example, the first surgeon can be a resident and the second surgeon can be a supervising surgeon. The guidance can include, for example, controlling a pointer that is overlaid on the direct view of the surgical field, drawing a desired contour of an incision that is overlaid on the direct view of the surgical field, controlling a virtual 3D model of a tool that is overlaid on the direct view of the surgical field, and so on. In these embodiments, the surgeon controlling the overlay (e.g., the supervising surgeon) can control the overlay (e.g., move a virtual cursor, draw a virtual contour, control a virtual 3D model of a tool, etc.) by viewing the overlay superimposed with the live video from the exoscope (or microscope), while the same overlay is also displayed to the surgeon that is directly viewing the surgical field (e.g., and not the exoscope video), such that the overlay is accurately superimposed with the direct view of the surgical field. In other words, the location of the overlay viewed by the first user with respect to the direct view of the anatomy is identical to the location of the overlay viewed by the second user controlling the overlay with respect to the anatomy, as it appears in the exoscope video. In these embodiments, the processor generates the overlay viewed by the first user based on the overlay controlled by the second user, based on the relative pose between exoscope (or microscope) and the patient, and based on the relative pose between the HMD and the patient: the overlay is “copied” from the exoscope image coordinate system to the patient coordinate system based on the relative pose between exoscope (or microscope) and the patient, and is then displayed via the first user's HMD based on the relative pose between the HMD and the patient. In some of these embodiments, the processor can also use a 3D model of the surgical field when generating the overlay. For example, when the second user controls the overlay with respect to a 2D live image, the 3D model can be used to locate the overlay at the correct depth (for example, by assuming that a virtual cursor or incision marking is intended to touch the anatomy). The 3D model can be determined from registered imaging data, from the stereoscopic exoscope (or microscope) image, and/or from a 3D sensor (e.g., a time-of-flight sensor, a structured light sensor, or any other type of 3D sensor embedded in the exoscope or in the HMD).

In some embodiments, the second user controls the overlay by viewing the overlay superimposed with a live image of the surgical field that is generated by a camera embedded in (e.g. integrated in) or attached to the HMD donned by the first user. In these embodiments, the processor “copies” the overlay from the camera image coordinate system to the patient coordinate system based on the relative pose between HMD and the patient, and continuously updates the overlay based on the live relative pose (e.g., when the overlay, for example, is a marking on the anatomy, the marking stays at its location when the first user moves relative to the patient).

In some embodiments, the direct view of the surgical field can be augmented with one or more virtual windows. For example, in minimally invasive surgery, and/or during percutaneous procedures, guidance information such as virtual 3D segmented anatomy can be displayed only within the borders of a virtual window. Using a virtual window can improve the user's depth perception by providing occlusion and parallax cues. In some embodiments, the virtual window conforms to the shape of the anatomy. For example, in a minimally invasive spine procedure, the virtual window can conform to the surface of the skin. In these embodiments, the shape of the anatomy can be derived, for instance, from registered imaging data, from the stereoscopic cameras of the exoscope, or from a 3D sensor or a depth sensor (e.g., a time-of-flight sensor or a structured light sensor) that is embedded in the exoscope or in the HMD.

In some embodiments, the direct view of the surgical field can be augmented with a virtual representation of the exoscope (or microscope) image footprint (e.g., the footprint represents the area on the anatomy that is displayed in the exoscope live image). In these embodiments, the size and location of the overlay representing the footprint relative to the anatomy is dynamically updated based on the current magnification level of the exoscope image and based on the current pose of the exoscope relative to the patient. The image footprint can represent the images viewed by the user and/or the images acquired by the exoscope (e.g., when the exoscope comprises an optical zoom lens or lenses, the images acquired by the exoscope and the images viewed by the user can be one and the same, but when the exoscope comprises digital magnification, the images viewed by the user can be an ROI within the images acquired by the exoscope). The overlay representing the footprint is also dynamically updated based on the HMD pose relative to the patient. Using the exoscope footprint overlay can improve the user's spatial orientation. For example, in minimally invasive spine surgery, when using an exoscope (or microscope) to view an area of interest through a tubular retractor, the exoscope footprint overlay can be superimposed on the direct view of the patient along with a 3D model of the patient's spine. This can facilitate the user's understanding regarding the exact anatomical location that is seen in the exoscope image. The virtual representation of the footprint can comprise the image itself (e.g., which will appear very small, as it is scaled to the actual anatomy as seen from the surgeon's perspective), or just as a symbol representing the area acquired in the image.

In some embodiments, a virtual representation of an image footprint can be generated when the image is acquired by tracked devices other than the exoscope, such as an endoscope or laparoscope (or generally any suitable camera assembly). In some embodiments, the virtual representation of an image footprint can be superimposed on a 3D model of the anatomy that is not overlaid on the direct view of the surgical field. For example, it can be displayed as part of navigation guidance, not necessarily while the user's view is directed towards the surgical field. The 3D model with the overlaid footprint can be magnified, so for instance, when the footprint comprises the live image, the user can see details in the live image laid out on the 3D model, each pixel at its corresponding depth. In some embodiments, the navigation guidance including the image footprint superimposed on a 3D model of the anatomy is displayed as a picture-in-picture (PIP) on the live image from the exoscope cameras.

In some embodiments, the direct view of the surgical field can be augmented with guidance information that is generated at least based on images acquired by the exoscope (e.g., by the cameras in the exoscope or microscope that acquire the video that the surgeon can view when operating, or by other cameras or sensors included in the exoscope or microscope). For example, the exoscope can include a near infrared camera for enhancing the view of blood vessels and/or tumors (e.g., as described herein above). The image from the near infrared camera can be, for instance, processed to identify and/or segment the enhanced elements, and a virtual representation of the enhanced elements can be superimposed with the direct view of the surgical field (e.g., based on the relative poses between the exoscope and the patient and between the HMD and the patient). As another example, the guidance information can be based on data acquired by an OCT device that is fixed to, or integrated with the exoscope (or microscope). The OCT device can be used, for instance, for detecting residual tumor tissue during tumor surgery (for example during brain surgery). In some embodiments, images generated by the OCT device can be displayed in a picture-in-picture format over the exoscope image or over the direct view of the surgical field, while concurrently overlaying a symbol indicating the OCT scan line (or area) on the exoscope image or on the direct view of the surgical field. In some embodiments, machine learning is employed for automatic classification of healthy vs. tumor tissue, and the overlay includes an indication highlighting residual tumor tissue (e.g., either on the live image from the exoscope or on the direct view of the surgical field). It is noted that some of these embodiments can be implemented without a tracker, and some of these embodiments can be implemented with any tracker that tracks the exoscope and the HMD relative to the patient, and not necessarily with the modular optical tracker.

In some embodiments, the direct view of the surgical field can be augmented with verification symbols as described in U.S. Pat. No. 10,482,614. Such verification symbols can indicate the validity of guidance overlays (e.g., the accuracy of the location of the overlays relative to the anatomy).

In some embodiments, the direct view of the surgical field can be augmented while taking into consideration occlusion by elements in the surgical field, as described in US20220354691. For example, an overlay can appear as if a tool or a hand is occluding it by not superimposing the overlay on the tool and the hand. In this example, the relative pose between the tool and the HMD can be known, for instance, if the tool is tracked, and this (e.g., in addition to the known pose of the HMD relative to the patient) can allow system 800 to generate the overlay such that it is not superimposed over the tool. The relative pose between the tool (or hand, or any other occluding element) and the HMD can also be known, for instance, from a camera and/or a 3D sensor (e.g., time-of-flight sensor, structured light sensor, stereoscopic camera, etc.) embedded in the HMD. In another example, the occlusion can be determined based on segmenting a live image from a camera embedded in the HMD, without determining a relative pose between the occluding elements and the HMD. In some embodiments, a 3D model of the surgical field (e.g., including tools and optionally hands holding the tools and other occluding elements) can be determined and updated in real-time based on the images from the stereoscopic exoscope cameras or based on a 3D sensor embedded in the exoscope. The model can be determined in the patient coordinate system, based on the pose of the exoscope relative to the patient, and the occlusion can be based on this model and based on the pose of the HMD relative to the surgical field (e.g., and hence also relative to the 3D model).

It is noted that in some embodiments, one or more of the augmentations on the direct view can also be implemented, for example, with systems such as system 700 or system 900, or any system including a see-through HMD.

In some embodiments, overlays can be superimposed on the direct view of the surgical field such that they appear below the surface of the anatomy. For example, the overlay can be displayed with a brightness that allows the surface of the anatomy that is above the element represented by the overlay to be viewed concurrently with the overlay. Combining the partial overlay transparency (e.g., due to the low brightness) with a correct left-right disparity of the overlay (e.g., the difference between the overlay location in image displayed to the left eye of the HMD user and the overlay location in image displayed to the right eye of the HMD user), which is different than the left-right disparity of the surface of the anatomy, as directly viewed by the user (e.g., since the overlay represents an element that is beneath the surface of the anatomy), can cause the overlay to appear as being at the correct depth below the surface of the anatomy. In some embodiments, when the augmented element represents a 3D object (e.g., such as a tumor, a vertebra, etc.), the overlay displayed to the left and right eyes of the HMD user can be each rendered from a different point of view (e.g., the two points of view corresponding to the points of view of the user's eyes), further enhancing the depth perception of the overlay relative to the surface of the anatomy. In some embodiments, overlays can be displayed within a virtual window. When using a virtual window (e.g., a virtual window which is also part of the overlay), the virtual window is displayed with a left-right disparity that matches that of the surface of the anatomy (e.g., as viewed directly by the user), and the surface of the anatomy within the borders of the virtual window can be completely occluded by the overlay (e.g., by displaying the overlay with a high brightness. Note that in these cases the augmented element is typically displayed within a substantially uniform background filling the area within the virtual window). In some embodiments, however, both a virtual window and a semi-transparent overlay (e.g., overlay displayed with low brightness) can be used together, generating an effect of looking inside the anatomy through a “dirty” window. These embodiments can be preferred when it is desired to see both the surface of the anatomy and the augmented element (for instance, when the anatomy is the cortex, and the augmented element is a tumor underneath the surface of the cortex). The virtual window further enhances the depth perception, since the augmented element can be viewed only within the borders of the virtual window (e.g., a parallax effect is generated due to the different depths of the virtual window and the augmented element, when the user moves relative to the patient). The use of a virtual window without the semi-transparent overlay (e.g., displaying the overlay and the uniform background within the virtual window in high brightness) can be preferred when there is no interesting information on the surface of the anatomy, for instance in a posterior minimally invasive spine surgery (e.g., when the surface of the anatomy is the patient's skin, and the augmented elements are vertebrae and other elements under the skin). It is noted that the augmented elements may represent not only anatomical tissues but also other physical elements such as, for example, surgical tools and implants, and virtual elements such as, for example, various preplanned guidance information.

In some embodiments, the direct view of the surgical field (e.g., through the transparent or semi-transparent HMD optics, when the HMD is an optical see-through HMD, or via the video from the forward-looking camera/s in the HMD, when the HMD is a video see-through HMD) is augmented with guidance information that is generated based at least on one or more of the relative poses between pairs selected from the HMD, the exoscope (or microscope), and the patient. In some embodiments, the rendering of the guidance information is done from the perspective of the eyes of the user donning the HMD, based on the relative pose between the HMD and the patient. In some embodiments, the guidance information is augmented on the direct view of the surgical field as a picture-in-picture (e.g., not necessarily in an accurate scale, location and orientation with respect to the anatomy). For example, standard navigation planes, including a virtual representation of a tracked tool (such as tool 80d in FIG. 8), accurately overlaid on axial and sagittal planes generated from the patient's imaging data, can be overlaid on the direct view of the surgical field at a fixed position relative to the 2D coordinate system of the HMD display (e.g., as a picture-in-picture). As another example, the position of the navigation planes (e.g., displayed in one or more pictures-in-picture) relative to the 2D coordinate system of the HMD display can be dynamically updated so as to not occlude the direct view of an area of interest in the surgical field. The area of interest can be known, for instance, based on a designation by the user, based on a preplanning information, based on a location a tooltip, based on an area currently imaged by the exoscope cameras (or other tracked cameras), or based on a displayed magnified area (e.g., when the exoscope image is viewed). In some embodiments, the guidance information is augmented on the direct view of the surgical field in a virtual screen (or screens) at a fixed position relative to the patient coordinate system. For example, standard navigation screens can appear as fixed to the patient and unchanged when, for example, the HMD user views the surgical field from different perspectives. In another example, the location of the virtual screens can be fixed to the patient but their orientation can dynamically change according to the surgeon's perspective (e.g., so that they always face the surgeon). In some embodiments, the guidance information is augmented on the direct view of the surgical field in a virtual screen (or screens) at a position that changes according to the surgeon's location relative to the patient. In these embodiments, the guidance information (e.g., the navigation guidance) can be generated at least based on the relative pose between the tracked tool and the patient. In some of these embodiments, the guidance information can also be generated based on the relative pose between the HMD and the patient.

In some embodiments, the exoscope (or microscope) position and/or orientation relative to the patient and/or relative to the HMD can be controlled based on one or more relative poses between objects selected from the exoscope, the HMD, and the patient. In some embodiments, the exoscope (or microscope) position and/or orientation relative to the patient and/or relative to the HMD can be controlled based on one or more relative poses between a tool and an object selected from the exoscope, the HMD, and the patient. In some embodiments, the exoscope (or microscope) position and/or orientation is controlled based on one or more relative poses and an enablement input from a user (e.g., the surgeon donning the HMD or another user). In some of these embodiments, the enablement input from the user can be one or more inputs selected from a footswitch input, a voice input, a virtual menu input (e.g., a virtual menu displayed via the HMD and operated by the surgeon), a hand gesture input, an eye gesture (e.g., when the HMD includes an eye tracker), and a touchscreen input (e.g., a touchscreen operated by an operating room staff member). In some embodiments, the exoscope (or microscope) position and/or orientation can be adjusted based on one or more relative poses only while a continuous enablement input from the user is received. For example, the exoscope position and/or orientation can be enslaved to the HMD motion only as long as a specific footswitch enablement pedal or button is continuously pressed. In some embodiments, the exoscope (or microscope) position and/or orientation can be adjusted based on one or more relative poses after a one-time enablement input from the user is received. In these embodiments, enablement can be discontinued either automatically, or by an input from a user. For example, one voice command can start the enablement of enslaving the exoscope position and/or orientation to the HMD motion, and another voice command can stop the enablement. In another example, a footswitch input can start enablement of enslaving the exoscope position and/or orientation to the HMD motion, and the enablement can stop automatically when no HMD motion is detected for a predefined period of time (such as, for example, 0.5 second). It is noted that in the embodiments herein it is assumed that the exoscope is suspended by a robotic arm or by a motorized arm allowing automatic adjustment of one or more degrees of freedom of the exoscope (or microscope) position and orientation.

In some embodiments, the exoscope (or microscope) orientation can be automatically adjusted so as to always face the surgeon. For example, during head surgery the exoscope can be suspended above the patient's head, and the surgeon may stand at different locations relative to the patient. When the exoscope comprises two cameras for acquiring a stereoscopic image, the surgeon donning the HMD and viewing the stereoscopic image can prefer that the exoscope is correctly rotated such that the stereoscopic image is acquired from the same perspective as the surgeon's perspective relative to the patient. The surgeon can opt to manually rotate the exoscope every time he or she moves to a different location, or alternatively, the integrated visualization and guidance system 800 can automatically rotate the exoscope based on the relative pose between the surgeon (e.g., the HMD donned by the surgeon) and the exoscope (e.g., with or without an enablement as described above).

In some embodiments, the exoscope (or microscope) orientation relative to the surgical field can be enslaved to the HMD while being locked to a point in the surgical field. For example, the surgeon can designate a point in the surgical field (e.g., by pointing with a tool, by controlling a virtual cursor overlaid on the live image with head gestures), press an enablement button in a footswitch, and rotate his or her head. In response, the integrated visualization and guidance system 800 can automatically rotate the exoscope to follow the change in the orientation of the HMD (e.g., based on the relative pose between the HMD and the patient), while keeping the designated point in the center of the live image. Once the surgeon releases the enablement button, the enslavement is discontinued and the surgeon can freely rotate his or her head to a comfortable angle. In this example, the surgeon can skip the step of designating the point, and the system will select a default point (e.g., such as the point appearing in the center of the live image). It is noted that when the point to be “locked” to is selected in the live image (e.g., and not by a tool pointing to a location in a 3D coordinate system, such as the coordinate system of the patient tracking unit), the integrated visualization and guidance system 800 can determine the 3D location of the point based on the depth of the point in the stereoscopic image (e.g., the distance between the exoscope and the point), and based on the relative pose between the exoscope and the patient. Once the 3D location of the point is known, the exoscope position and orientation can be correctly adjusted so as to follow the HMD rotation while keeping the point in the center of the image. In this example, the distance between the exoscope and the surgical field can be kept unchanged throughout the motion. In some embodiments, the control over the exoscope orientation or position by enslavement to HMD movements can be user-configurable. For example, the user can configure the system to enhance HMD rotation, for instance to rotate the exoscope by 20 degrees per second when the HMD is rotated by 10 degrees per second. As another example, the user can configure the system to respond to specific head gestures by adjusting exoscope rotation and to other head gestures by adjusting exoscope position (e.g., with or without enablement as described above.

In some embodiments, the exoscope (or microscope) distance to the surgical field can be enslaved to the HMD position and/or orientation while being locked to a point in the surgical field. For example, the surgeon can press an enablement button in a footswitch, and rotate his or her head up or down. In response, integrated visualization and guidance system 800 can increase or decrease the exoscope distance from the surgical field while keeping a selected point in the center of the live image and keeping the exoscope viewing angle unchanged. In this example, the system can automatically adjust the focus to compensate for the distance change. In some embodiments, the exoscope (or microscope) pose relative to the surgical field (e.g., relative to the patient tracking unit) can be enslaved to a tracked tool. For example, the surgeon can press an enablement button in a footswitch, and in response, integrated visualization and guidance system 800 can adjust the exoscope pose such that the exoscope optical axis (e.g., the vector between the optical axes of the two exoscope cameras) is aligned with the tool. In this example, the distance between the exoscope and the surgical field can be kept unchanged when the exoscope pose is adjusted. In some embodiments, the surgeon can save a current exoscope pose (e.g., “bookmark” the current pose), and later return to a saved pose. For example, the surgeon may choose one of several saved poses by operating a virtual menu.

In some embodiments, the exoscope (or microscope) position and/or orientation relative to the patient can be controlled based on one or more relative poses between a tool and an object selected from the exoscope, the HMD, and the patient. For example, when a cannula is used in a procedure, the surgeon may hold a tool (e.g., navigated pointer) in alignment with an axis of the cannula, and instruct the exoscope to align itself with the tool (for example, by pressing a footswitch, by voice command, or by operating a virtual menu via head gestures). The exoscope then aligns itself to the cannula axis based on the relative pose between the tool and the exoscope. In another example, a tracking unit is directly attached to the cannula (e.g., the cannula is a navigated tool), and the surgeon can instruct the exoscope to align itself with the cannula without needing to hold a pointer. In these examples, the alignment can be either on-demand or continuous, with or without an enablement as described above. In some embodiments, exoscope alignment with a cannula (or with any tool) can be implemented based on the exoscope image alone, without tracking the cannula. This can be implemented, for example, by utilizing machine learning and/or image processing algorithms that identify the cannula (or tool) in the image, allowing to automatically reposition and reorient the exoscope to center the cannula in the image and/or to align the exoscope optical axis with the cannula axis (e.g., typically the exoscope is a stereoscopic exoscope having two optical axes, so the midline between the two axes can be aligned with the cannula/tool). In some embodiments, the exoscope can automatically align itself with a surgical corridor generated by retractors and/or distractors. This can be implemented, for example, by utilizing machine learning and/or image processing algorithms that identify the retractors and/or distractors and align the exoscope in an optimal angle for viewing the tissue at the end of the corridor. In some embodiments, machine learning and/or image processing algorithms can allow for automatic focusing on the tissue or near a tooltip, and for optimizing the range of depths that are in focus to facilitate the work through the corridor (or cannula). In some embodiments, when the exoscope position and/or orientation is automatically controlled (e.g., with or without an enablement by the user) based on tracking or based on the exoscope image alone, the system can indicate the new determined exoscope position and/or orientation to the surgeon prior to moving the exoscope (e.g., via the motorized arm), so as to allow the surgeon to abort the movement if the automatic angle determination was erroneous. Notifying the surgeon can be implemented, for example, by overlaying a symbol indicating the new exoscope position and/or orientation on the exoscope image and/or on the direct view of the surgical field. It is noted that some of these embodiments can be implemented without in-out/out-in tracking or without tracking at all.

Reference is now made to FIG. 9, which presents a schematic illustration of an integrated visualization and guidance system 900 utilizing an inside-out/outside-in optical tracking system for determining a relative pose between several objects, according to some embodiments. As shown in FIG. 9, a user (e.g., surgeon) 950, is wearing a head mounted display (HMD) 90a. HMD 90a (a first object) is associated with a first optical tracking unit 902a and a second optical tracking unit 902b, both embedded within HMD 90a. Patient 952 is shown with third optical tracking unit 902c associated with a body portion thereof. Tracking unit 902c is attached to the patient's posterior superior iliac spine by a pin, such that tracking unit 902c is essentially fixed with respect to the patient's lumbar spine (second object 90b). Further shown is a robotic arm 90c (third object), which is associated with five tracking units 902d-902h (three of them are shown in FIG. 9). All tracking units 902a-902h include at least one optical sensor and at least one visual indicator. In the example depicted in FIG. 9, patient tracking unit 902c includes three sensors and three visual indicators, as described herein below, and all other tracking units include one sensor and one visual indicator. In the exemplary configuration illustrated in FIG. 9, the tracking system facilitates the determination of the relative poses between the first, second and third objects.

As shown in FIG. 9, a pair of tracking units selected from tracking units 902d-902h are employed together with tracking unit 902c in tracking the relative pose between the robotic arm and the patient. The employment of the specific pair of tracking units, and the specific optical sensor to be employed within this pair, may be determined based on the availability in the field of view, based on history (i.e., which one was used in previous cycles), or based on optimizing accuracy and occlusion. Thus, in such a setting, one pair of tracker units selected from tracking units 902d-902h can be utilized for tracking the patient tracker (e.g., for tracking the relative pose between the exoscope and the patient's sacrum) during one part of the procedure, and another pair of tracker units can be utilized for tracking the patient tracker during another part of the procedure (e.g., when the location of the robotic arm relative to the patient is changed). Similarly, the selection of the optical sensor within the selected pair of tracking units to be employed for tracking can also be dynamically switched.

It is noted that tracking units 902d-902h are embedded within (or fixed to) the robotic arm such that the distances between pairs of adjacent tracking units support accurate tracking between the robotic arm and the patient. For example, the tracking units may be fixed to the robotic arm with distances of, for example, 5-10 cm therebetween, corresponding to the distance between the robotic arm and the patient tracking unit, which is typically 10-40 cm from the robotic arm during surgery. Thanks to the small form factor of the modular tracking units, embedding (or fixing) the tracking units within the robotic arm is easy and the increase in size and weight of the robotic arm due to the tracking units is negligible. This is especially true when the tracking units are embedded within the robotic arm and therefore do not require dedicated housing and power supply (e.g., a battery). In these cases, for example, a tracking unit can consist of only a small PCB comprising the optical sensor (e.g., including its optics) and the LED.

Further in FIG. 9, HMD tracking units 902a and 902b are employed together with one of the robotic arm tracking units 902d-902h to track the relative pose between the HMD and the robotic arm. HMD tracking units 902a and 902b are also employed together with the patient tracking unit 902c to track the relative pose between the HMD and the patient. Similar to as detailed above, the employment of the specific optical sensor may be determined based on the availability in the field of view, based on history (i.e., which one was used in previous cycles), or based on optimizing accuracy and occlusion. Thus, in such a setting, the sensor of one of the HMD tracker units can be utilized for tracking the patient tracker, and the sensor of the other HMD tracker unit can be utilized for tracking the robotic arm, while optionally, dynamically switch roles therebetween.

Patient tracking unit 902c in the example depicted in FIG. 9 comprises three sensors and three visual indicators, so as to provide a FOV large enough to cover both the HMD and the robotic arm. In the example depicted in FIG. 9, only one sensor and one visual indicator in the patient tracking unit (e.g., the sensor and visual indicator generally facing the robotic arm) are employed for tracking the relative pose between the patient and the robotic arm, and another pair of sensor and visual indicator in the patient tracking unit (e.g., the sensor and visual indicator generally facing up) are employed for tracking the relative pose between the patient and the HMD.

FIG. 9 further shows a tool (e.g., a power drill), fourth object 90d, and tracking unit 902i that is fixed to the tool. In the example depicted in FIG. 9, tool 90d is guided by the robotic arm. In general, the robotic arm can be employed to guide a tool, for example to limit a motion of a tool such as a drill to a determined trajectory or, to limit a motion of a tool such as a saw blade to a determined plane. Alternatively or additionally, the robotic arm can be employed to hold a tool and directly control its movement. In some embodiments, integrated visualization and guidance system 900 utilizes in-out/out-in tracking for tracking a relative pose between a tool, such as tool 90d, and each of the HMD, the robotic arm, and the patient, as described above. In these embodiments, the tool can be fixed with a tracking unit comprising one or more optical sensors and one or more visual indicators. For example, tracking unit 920i can comprise one pair of sensor and visual indicator facing up, and one pair of sensor and visual indicator facing down, thus facilitating tracking the relative poses between the tool and each of the HMD and the robotic arm (e.g., the relative pose between the tool and the patient can be derived from any of these poses, as the relative poses between the HMD and the patient, and between the robotic arm and the patient, are known). In some embodiments, integrated visualization and guidance system 900 utilizes out-in tracking for tracking the relative poses between each of the HMD, the patient, and the robotic arm and a tool, such as tool 90d. In these embodiments, tracking unit 902i can comprise visual indicators alone (e.g., reflectors or light emitting, without an optical sensor). In these embodiments, out-in tracking can be implemented using the optical sensors of the same robotic arm tracking units that are also employed for in-out/out-in tracking between the robotic arm and the patient, and/or between the robotic arm and the HMD. The optical sensors of the HMD tracking units, and a selected one of the optical sensors of the patient tracking unit 902c (e.g., dynamically selected based on availability of the tool tracking unit in the FOV thereof) can also be used. In these embodiments, out-in tracking can be implemented as described herein above with respect to FIG. 8.

In some embodiments, during part of a surgical procedure information relating to the robotic arm and/or a tool which is guided or held by the robotic arm is displayed to the surgeon via the HMD. In some embodiments, during part of a surgical procedure information relating to the robotic arm and/or a tool which is guided or held by the robotic arm is not displayed via the HMD. In some embodiments, information relating to the robotic arm and/or a tool which is guided or held by the robotic arm is displayed and/or not displayed based on the head direction of the surgeon donning the HMD. For example, when the surgeon's head is directed towards the surgical field, display of such information can be discontinued to allow the surgeon to view the surgical field directly (e.g., through the semi-transparent or transparent HMD optics, when the HMD is an optical see-through HMD, or via the video from the forward-looking camera/s in the HMD, when the HMD is a video see-through HMD). As another example, when the surgeon's head is directed above the surgical field, display of such information can be resumed. In some embodiments, HMD directions (e.g., surgeon's head directions) wherein information relating to the robotic arm and/or a tool which is guided or held by the robotic arm is displayed or not displayed can be configured by a user. In some embodiments, various types of information relating to the robotic arm and/or a tool which is guided or held by the robotic arm are displayed based on the head direction of the surgeon donning the HMD. In some embodiments, one type of information relating to the robotic arm and/or a tool which is guided or held by the robotic arm is displayed when the surgeon's head is directed towards the surgical field, and another type of information relating to the robotic arm and/or a tool which is guided or held by the robotic arm is displayed when the surgeon's head is directed away from the surgical field. In some embodiments, overlays configured to be superimposed on the direct view of the surgical field are displayed when the HMD is directed towards the surgical field. In some embodiments, when the HMD is a video see-through HMD, overlays on the direct view of the surgical field are overlaid on a live image from a camera (or cameras) embedded in (or attached to) the HMD. In some embodiments, when the HMD is a video see-through HMD, the live video from the camera (or cameras) embedded in the HMD is displayed when the HMD is directed towards the surgical field.

In some embodiments, information relating to the robotic arm and/or a tool which is guided or held by the robotic arm can include the location of the tool, the location of the tool guide (e.g., as defined by the end effector of the robotic arm), and/or the location of a trajectory or plane defined by the tool guide, relative to 2D and/or 3D images generated from the patient's imaging data. In some embodiments, when the surgeon's head is directed towards the surgical field, information relating to the robotic arm and/or a tool which is guided or held by the robotic arm can include a virtual representation of the tool and a virtual representation of the anatomy, both registered to the patient's anatomy (e.g., displayed as in-situ augmented reality). In some embodiments, when the surgeon's head is directed towards the surgical field, information relating to the robotic arm and/or a tool which is guided or held by the robotic arm can include previously determined entry points, trajectories, planes, and/or any other planning information. In some embodiments, planning can be performed by the surgeon with the help of a tracked instrument (such as a tracked pointer) with or without the robotic arm being in the surgical field. In these embodiments, the planned guidance can be locked relative to the patient and can be overlaid on the direct view of the surgical field. Once the planning is complete the surgeon can direct the robotic arm to automatically align the guide and/or the tool with the plan, and can then verify, by directly viewing the surgical field, that the robotic arm is indeed correctly aligned with the plan. For example, the surgeon can verify that a safe corridor for using a power drill during pedicle screw placement is indeed aligned with a planned trajectory.

In some embodiments, more than one tracking unit is fixed relative to the patient. For example, in a one-level spine procedure involving two vertebrae, a tracking unit can be directly fixed relative to each of the two vertebrae (e.g., when the tracking unit comprises two parts, the functional part is fixed relative to each vertebra). Once the two tracking units are registered, each to its corresponding vertebra, the relative pose between the two vertebrae can be tracked. In this example, tracking the relative pose between the two vertebrae may obviate a need to use an imaging device to verify that the procedure was performed correctly. As another example, in a total joint replacement procedure, a tracking unit can be fixed to each of the bones connected by the joint. It is noted that fixing more than one tracking unit to a patient can be useful with or without the exoscope or the robotic arm of FIGS. 8 and 9. It is further noted that attaching multiple tracking units to a patient is facilitated by the small size of the tracking units described herein above.

In some embodiments, an integrated visualization and guidance system can comprise both an exoscope and/or an endoscope (as described above, for example, with respect to FIG. 8), and a robotic arm (as described above, for example, with respect to FIG. 9), in addition to the VGS capabilities (as described above, for example, with respect to FIG. 7).

In some embodiments, the VGS system and the integrated visualization and guidance systems described herein can allow the HMD user to switch between screens, and/or to invoke a picture-in-picture (PIP) to be superimposed with the screen displayed via the HMD. In some embodiments, the content of each of the available screens and PIPs can be preconfigured (e.g., before each procedure or by adjusting the user preferences). For example, the user can configure one screen to display live feed from a neuromonitoring system, a second screen to display live feed from the anesthesia machine, a third screen to display the patient file, and a fourth screen to serve as a picture archiving and communication system (PACS) monitor (e.g., allowing access to preoperative and intraoperative images). Similarly, the user can preconfigure the contents of various PIPs. Typically, during most of the procedure, the user views the default screens (e.g., screens displaying live video from the exoscope cameras and/or the endoscope camera, screens displaying navigation information and/or in-situ AR guidance). In some embodiments, the user can switch from a default screen to one of the preconfigured screens. Switching between screens can be done by any of the user interface options provided by the systems, such as by operating a footswitch, by head gestures, by voice commands, by operating virtual menus (e.g., menus displayed via the HMD), or by any combination of user interface options. For example, the user can enable screen switching via footswitch, and select a screen via head gestures. In some embodiments, the user can invoke a PIP to be superimposed with the screen currently displayed via the HMD. Invoking a PIP can be done by any of the user interface options provided by the systems, such as by operating a footswitch, by head gestures, by eye gestures, by voice commands, by operating virtual menus, or by any combination of user interface options. For example, the user can invoke a menu showing the various preconfigured PIPs as icons that are overlaid on the HMD image (e.g., HMD-stabilized overlay), navigate within the menu and select a PIP by head gestures (for example, moving the head changes which of the menu items is highlighted, and holding the head still selects the highlighted menu item). In some embodiments, the user can resize and reposition the PIP via any of the user interface options, including by a touchscreen input (e.g., a touchscreen operated by an operating room staff member).

As used herein, the term “object” relates to any type of live or inanimate object that can be tracked or be associated with a tracking unit. In some embodiments, the object is a subject or a patient. In some embodiments, the object is a tool. In some embodiments, the object is a head mounted display. In some embodiments, the object is a camera, a microscope, an exoscope, a robotic arm, an automated medical device, and the like.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. No feature described in the context of an embodiment is to be considered an essential feature of that embodiment, unless explicitly specified as such.

Although stages of methods, according to some embodiments, may be described in a specific sequence, the methods of the disclosure may include some or all of the described stages carried out in a different order. In particular, it is to be understood that the order of stages and sub-stages of any of the described methods may be reordered unless the context clearly dictates otherwise, for example, when a later stage requires as input an output of a former stage or when a later stage requires a product of a former stage. A method of the disclosure may include a few of the stages described or all of the stages described. No particular stage in a disclosed method is to be considered an essential stage of that method, unless explicitly specified as such.

Although the disclosure is described in conjunction with specific embodiments thereof, it is evident that numerous alternatives, modifications, and variations that are apparent to those skilled in the art may exist. Accordingly, the disclosure embraces all such alternatives, modifications, and variations that fall within the scope of the appended claims. It is to be understood that the disclosure is not necessarily limited in its application to the details of construction and the arrangement of the components and/or methods set forth herein. Other embodiments may be practiced, and an embodiment may be carried out in various ways.

The phraseology and terminology employed herein are for descriptive purpose and should not be regarded as limiting. Citation or identification of any reference in this application shall not be construed as an admission that such reference is available as prior art to the disclosure. Section headings are used herein to ease understanding of the specification and should not be construed as necessarily limiting.

1. An optical tracking system comprising:
a first, second, and third optical tracking units; and
one or more processors communicatively associated with the tracking units;
wherein each of the tracking units comprises an optical sensor and a visual indicator;
wherein the first and second tracking units are configured to be fixed relative to one another; and
wherein the one or more processors are configured to determine, when the first and second tracking units are fixed relative to one another, a relative pose between a first coordinate system, which is associated with the first and second tracking units, and a second coordinate system, which is associated with the third tracking unit, based at least on:
information indicative of a pose of each of the first and second tracking units in the first coordinate system;
a first data set indicative of at least one direction in the first coordinate system from the optical sensor of the first tracking unit and/or the optical sensor of the second tracking unit towards the visual indicator of the third tracking unit; and
a second data set indicative of two directions in the second coordinate system from the optical sensor of the third tracking unit towards the visual indicator of the first tracking unit and the visual indicator of the second tracking unit.

2. The system according to claim 1, wherein each of the first data set and the second data set comprises at least one of: blob data and image data.

3. The system according to claim 1, wherein the one or more processors are further configured to determine, based on the first and second data sets, at least one direction from at least one of the optical sensors towards at least one of the visual indicators.

4. The system according to claim 1, wherein the one or more processors are further configured to select from the first and second tracking units, whether both thereof, or which thereof, are used to obtain the first data set.

5. The system according to claim 1, wherein each of the optical sensors is selected from a pixel array sensor, a CMOS sensor, a CCD sensor, a pair of linear sensor arrays, a 2D lateral effect position sensor, a photodiode array position sensor, a segmented photodetector, a quadrant position-sensing detector, a position-sensitive detector, and a light sensitive element.

6. The system according to claim 1, wherein each of the visual indicators comprises a light emitting diode (LED) or a reflector.

7. The system according to claim 1, wherein the tracking units are fabricated to be similar with respect to one or more of size, dimensions, type of optical sensor, type of visual indicator, type of communication unit, communication protocol, and/or functionality.

8. The system according to claim 1, wherein the first and second tracking units are configured to be fixed relative to one of: a head mounted display (HMD), a surgical microscope, a surgical exoscope, a surgical robot, a surgical robotic arm, an imaging device, a surgical light.

9. The system according to claim 1, wherein the third tracking unit is configured to be fixed relative to one of: a hand-held tool, a subject, an anatomical site, a camera, an endoscope, a laparoscope.

10. The system according to claim 1, wherein the information indicative of the relative pose between the first and second tracking units is predetermined.

11. The system according to claim 1, wherein the first and second tracking units are permanently attached to an object.

12. The system according to claim 1, wherein the one or more processors are further configured to implement a calibration stage wherein the relative pose between the first and second tracking units is computed.

13. The system according to claim 12, wherein the information indicative of a pose of each of the first and second tracking units in the first coordinate system comprises previously acquired first and second data sets.

14. The system according to claim 1, wherein the information indicative of a pose of each of the first and second tracking units in the first coordinate system is based on an accurate mounting mechanism.

15. An optical-based tracking method for determining a relative pose between (i) a first coordinate system, which is associated with a first tracking unit and a second tracking unit, and (ii) a second coordinate system, which is associated with a third tracking unit, each of the tracking units comprises an optical sensor and a visual indicator, the method comprising:
using the first tracking unit and/or the second tracking unit to obtain a first data set indicative of at least one direction in the first coordinate system from the optical sensor of the first tracking unit and/or the optical sensor of the second tracking unit towards the visual indicator of the third tracking unit;
using the third tracking unit to obtain a second data set indicative of two directions in the second coordinate system from the optical sensor of the third tracking unit towards the visual indicator of the first tracking unit and the visual indicator of the second tracking unit; and
determining the relative pose between the first coordinate system and the second coordinate system based at least on the first and second data sets and information indicative of a pose of each of the first and second tracking units in the first coordinate system;
wherein the first and second tracking units are fixed relative to one another.

16. The method according to claim 15, wherein each of the first data set and the second data set comprises at least one of: blob data and image data.

17. The method according to claim 16, further comprising determining, based on the first and second data sets, at least one direction from at least one of the optical sensors towards at least one of the visual indicators.

18. The method according to claim 15, further comprising selecting from the first and second tracking units, whether both thereof, or which thereof, are used to obtain the first data set.

19. The method according to claim 15, wherein each of the optical sensors is selected from a pixel array sensor, a CMOS sensor, a CCD sensor, a pair of linear sensor arrays, a 2D lateral effect position sensor, a photodiode array position sensor, a segmented photodetector, a quadrant position-sensing detector, a position-sensitive detector, and a light sensitive element.

20. The method according to claim 15, wherein each of the visual indicators comprises a light emitting diode (LED) or a reflector.

21-27. (canceled)
